(19) United States
(12) Patent Application Publication
(10) Pub. No.: US 2026/0028632 A1
(43) Pub. Date: Jan. 29, 2026

(54) NUCLEIC ACID AGENTS MODULATING FAS ISOFORMS

(71) Applicant: Hadasit Medical Research Services and Development Ltd., Jerusalem (IL)

(72) Inventors: Michal LOTEM, Jerusalem (IL); Ori STERN, Jerusalem (IL); Shiri KLEIN, Jerusalem (IL); Galit EISENBERG, Jerusalem (IL); Elad ZISMAN, Jerusalem (IL)

(73) Assignee: Hadasit Medical Research Services and Development Ltd., Jerusalem (IL)

(21) Appl. No.: 19/287,289

(22) Filed: Jul. 31, 2025

Related U.S. Application Data
(63) Continuation-in-part of application No. PCT/IL2024/050123, filed on Jan. 31, 2024.
(60) Provisional application No. 63/482,437, filed on Jan. 31, 2023.
(60) Provisional application No. 63/516,660, filed on Jul. 31, 2023.

(51) Int. Cl. C12N 15/113; A61K 40/11; A61K 40/42; A61P 35/00; C12N 9/22; C12N 15/11; C12N 15/90

(52) U.S. Cl. CPC C12N 15/1138; A61K 40/11; A61K 40/4224; A61P 35/00; C12N 9/226; C12N 15/11; C12N 15/907; C12N 2310/11; C12N 2310/20; C12N 2320/33

(57) ABSTRACT

The present disclosure relates to manipulation of splicing of the Fas (tumor necrosis factor (TNF) receptor superfamily, member 6) gene in non-naïve cell of the T lineage for enhanced skipping of exon 6. The manipulated non-naïve cells predominantly express a soluble form of Fas (sFas) and display reduced expression of the membranal Fas (mFas), increased cytokine secretion, increased expression of activation markers, increased cell survival, increased cytotoxicity, and/or reduced expression of exhaustion markers. The present disclosure further provides specific splicing modulatory agents comprising gene editing systems and/or splice switching antisense oligonucleotide (SSO), methods and therapeutic uses thereof.

### CROSS-REFERENCE TO RELATED APPLICATIONS

This application is a Continuation-in-Part of International Patent Application No. PCT/IL2024/050123, filed on Jan. 31, 2024, which claims priority to U.S. Application No. 63/516,660, filed Jul. 31, 2023, and U.S. Application No. 63/482,437, filed Jan. 31, 2023. The entire contents of these applications are incorporated herein by reference in their entirety.

### SEQUENCE LISTING

The instant application contains a Sequence Listing which has been submitted electronically in XML format is hereby incorporated by reference in its entirety. Said XML copy, created on Jan. 31, 2024, is named 269002000032_Seq_Listing and is 125,267 bytes in size.

### FIELD OF THE INVENTION

The present disclosure relates to immunotherapy. More specifically, the present disclosure provides improved cells of the T lineage that predominantly express the soluble form of Fas (sFas), compositions and methods thereof for the treatment of immune-related disorders, specifically cancer.

### BACKGROUND ART

References considered to be relevant as background to the presently disclosed subject matter are listed below:
Anderson K G, et al. Engineering adoptive T cell therapy to co-opt Fas ligand-mediated death signaling in ovarian cancer enhances therapeutic efficacy. J Immunother Cancer 2022; 10, pages 1-14.
Izquierdo J M, et al., Regulation of Fas Alternative Splicing by Antagonistic Effects of TIA-1 and PTB on Exon Definition. 2005, Molecular Cell, Vol. 19, 475-484.
WO 2019/040590.

Acknowledgement of the above references herein is not to be inferred as meaning that these are in any way relevant to the patentability of the presently disclosed subject matter.

### BACKGROUND OF THE INVENTION

Using the immune system as a weapon against cancer is widely implemented and is often a central component of first-line treatments. Adoptive-cell therapy (ACT), CAR-T cells, and checkpoint inhibitors are immunotherapies that often result in significant cancer regression. A substantial part of the success is attributed to the blockade of immune checkpoint receptors and activation of stimulatory receptors.

During the transcription process, the introns of the pre-mRNA are spliced out, and the exons are joined together, resulting in the mature mRNA. In 95% of the genes, side by side with the constitutive mRNA form, skipping over exons (or part of them) can lead to a different configuration of mRNA, significantly increasing the diversity of proteins. This process is called alternative splicing (AS). The function of the alternative transcripts sometimes diverts from the constitutive form in direction and magnitude.

FAS (CD95, TNFRSF6) is one of the best-known and studied members of the TNF receptors superfamily (TNFRSF), expressed in abundant tissues, including the gastrointestinal tract, the respiratory system, and lymphoid tissues. FAS is mainly known for its death signal transduction following FAS ligand (FASL) binding. However, it also has other functions, e.g., it takes part in the differentiation of naïve T to memory T cells. FAS is robustly expressed on T cells and has an apoptosis-inducing role during T cell development, and the immune response.

WO 2019/040590 discloses compositions and methods for maintaining or increasing the number of naïve T cells in a T cell population, e.g., by delivering ex vivo an oligonucleotide that inhibits the interaction of FAS-AS 1 with RBM5 to a T cell population comprising naïve T cells. Such compositions and methods are useful, e.g., in adoptive T cell therapies including chimeric antigen receptor (CAR) T cell therapies.

### SUMMARY OF THE INVENTION

A first aspect of the present disclosure relates to a non-naïve cell of the T lineage manipulated, modulated and/or modified for enhanced skipping of exon 6 of the Fas (tumor necrosis factor (TNF) receptor superfamily, member 6) gene, or a cell population comprising at least one of the cell/s of the present disclosure. Specifically, the non-naïve manipulated cell of the T lineage in accordance with the present disclosure is manipulated and/or modified such that it predominantly expresses a soluble form of Fas (sFas, also referred to herein as FASΔEX6).

A further aspect of the preset disclosure relates to a composition comprising at least one of: (a), at least one non-naïve cell of the T lineage manipulated for enhanced skipping of exon 6 of the Fas gene, or a cell population comprising at least one of said cell. The non-naïve manipulated cell of the T lineage according to the present disclosure predominantly expresses sFas. In yet some alternative or additional embodiments, the disclosed composition may further comprise and/or alternatively comprise (b), at least one splicing modulating agent or any vector, vehicle, matrix, nano- or micro-particle or composition comprising the at least one agent, the splicing modulating agent comprises at least one nucleic acid molecule that targets at least one target nucleic acid sequence participating directly or indirectly in at least one splicing event of the Fas gene. In some embodiments, the composition optionally further comprises at least one of pharmaceutically acceptable carrier/s, diluent/s, excipient/s and additive/s.

A further aspect of the present disclosure relates to a method for treating, preventing, ameliorating, inhibiting or delaying the onset of a pathologic disorder in a mammalian subject. The methods disclosed herein comprises the step of administering to the subject an effective amount of at least one of: (a), at least one non-naïve cell of the T lineage manipulated for enhanced skipping of exon 6 of the Fas gene, or a cell population comprising at least one of the manipulated cell/s. The non-naïve manipulated cell of the T lineage used by the disclosed methods predominantly expresses sFas. In yet some further embodiments, the methods disclosed herein may further, or alternatively administer (b), at least one splicing modulating agent or any vector, vehicle, matrix, nano- or micro-particle or composition comprising the at least one agent. The splicing modulating agent used by the disclosed methods comprises at least one nucleic acid molecule that targets at least one target nucleic acid sequence participating directly or indirectly in at least one splicing event of the Fas gene. Still further, the disclosed methods may further, or alternatively administer (c) a composition comprising the non-naïve cell/s of the T lineage of (a) and/or the at least one splicing modulating agent of (b). In some particular embodiments, the therapeutic methods disclosed herein may be useful for the treatment of neoplastic disorders.

A further aspect of the present disclosure relates to a therapeutically effective amount of at least one of (a) at least one non-naïve cell of the T lineage manipulated for enhanced skipping of exon 6 of the Fas gene, or a cell population comprising at least one of the cell/s. It should be noted that the non-naïve manipulated cell of the T lineage predominantly expresses sFas. Still further, in some embodiments of the disclosed use, the cell/s optionally further expresses at least one receptor molecule.
(b) at least one splicing modulating agent or any vector, vehicle, matrix, nano- or micro-particle or composition comprising the at least one agent. Such splicing modulating agent comprises at least one nucleic acid molecule that targets at least one target nucleic acid sequence participating directly or indirectly in at least one splicing event of the Fas gene; and/or
(c) a composition comprising the cell/s of (a) and/or the at least one splicing modulating agent of (b), for use in a method for treating, preventing, ameliorating, inhibiting or delaying the onset of a pathologic disorder in a mammalian subject. In some embodiments, the disclosed methods are particularly useful for the treatment of neoplastic disorders.

Thus, in some particular aspects, the disclosure provides therapeutic effective amount of at least one splicing modulating agent and/or the manipulated cells of the T lineage for use in a method for treating, inhibiting, preventing, ameliorating or delaying the onset of at least one neoplastic disorder in a subject.

A further aspect provided by the present disclosure relates to a method for improving activity and/or survival of at least one cell of the T lineage. The method disclosed herein may comprise the step of contacting at least one cell with an effective amount of at least one splicing modulating agent comprising at least one nucleic acid molecule or any vector, vehicle, matrix, nano- or micro-particle or composition comprising the at least one agent. It should be noted that the at least one nucleic acid molecule of the splicing modulating agents provided by the present disclosure, may target at least one target nucleic acid sequence that participates directly or indirectly in at least one splicing event of the Fas gene, thereby enhancing skipping of exon 6.

A further aspect of the present disclosure relates to a method of enhancing expression of soluble Fas (sFas) and/or inhibiting expression of membrane FAS (mFas) in a cell of the T lineage. Such method comprises the step of contacting the cell with at least one splicing modulating agent comprising at least one nucleic acid molecule or any vector, vehicle, matrix, nano- or micro-particle or composition comprising at least one agent. Specifically, any of the splicing modulating agents as disclosed by the present disclosure.

A further aspect of the present disclosure relates to at least one splicing modulating agent comprising at least one nucleic acid molecule or any vector, vehicle, matrix, nano- or micro-particle or composition comprising at least one agent, in accordance with the present disclosure. More specifically, the disclosed splicing modulating agent comprises at least one of (a), at least one oligonucleotide comprising a nucleic acid sequence complementary to at least part of the target nucleic acid sequence; and/or (b), at least one nucleic acid sequence comprising at least one gRNA that targets at least one protospacer within the target nucleic acid sequence, or any nucleic acid sequence encoding the gRNA, said gRNA guides at least one PEN to the target nucleic acid sequence in the Fas gene.

These and other aspects of the present disclosure will become apparent by the hand of the following drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

FIG. 1A-IF. Activated T cells undergo FASΔEX6 alternative splicing

FIG. 1A. Agarose gel electrophoresis showing the dynamics of mFAS and FASΔEX6 splicing transcripts during T cell activation.

Total mRNA was extracted from Naïve or activated CD4+ T cells isolated from PBMCs. Cells were activated with anti-CD3 and anti-CD28 coated beads for 72 hours, and mRNAs were extracted at time points as indicated above.

FIG. 1B. Graph showing the ratio of mFAS and FASΔEX6 transcripts. Transcripts from (FIG. 1A) were measured by ImageJ. The total FAS transcript was calculated as the sum of both transcripts.

FIG. 1C. Graph showing mFAS and FASΔEX6 splicing isoforms transcripts fold change following activation. Three healthy donors mRNA were extracted from activated PBMCs as indicated in (FIG. 1A). mFAS and FASΔEX6 splicing isoforms transcripts fold change was measured by Real-time qPCR. Expression of GUSB mRNA was used for normalization. One representative graph is shown.

FIG. 1D. Graph showing the percentage of mFAS and FASΔEX6 transcripts out of the total FAS transcript in Naïve or activated CD4+ T cells from (FIG. 1C). The total FAS transcript was calculated as the summary of both transcripts. The average results of three healthy donors are represented. Error bars represent s.d.

FIG. 1E. Graph showing fold change of mFAS and FASΔEX6 transcripts after T-cell activation measured by qPCR as described in (FIG. 1A). mRNAs were extracted from bulk or activated CD8+ T cells isolated from PBMCs. The cells were activated for 24 h.

FIG. 1F. Graph showing fold change of mFAS and FASΔEX6 transcripts as described in (FIG. 1E). mRNAs were extracted from bulk or activated CD4+ T cells isolated from PBMCs.

FIG. 2A-2H. FAS Exon 6 skipping induced by splice switching antisense oligonucleotides (SSO)

FIG. 2A. Schematic representation of six SSOs that are targeting the 5′SS and 3′SS of FAS exon 6.

FIG. 2B. Flow cytometry analysis for activation and differentiation markers. Healthy donor PBMCs were activated with 1 ug/ml anti-CD3 and anti-CD28 antibodies for 48 hours prior to SSO electroporation. Flow cytometry analysis for activation and differentiation markers showed that all cells [97% (19.17+75.20+2.75)] were activated (CD25+ and/or CD69+), all cells are FAS+[99%].

FIG. 2C. FACS histogram of mFAS expression in PBMCs transfected with various SSOs. PBMCs of healthy donor from (FIG. 2B) were transfected by electroporation with the SSOs (SSO #1-#6, as denoted by SEQ ID Nos: 2, 4, 6, 8, 10 and 12, respectively) indicated in (FIG. 2A) or with SSO SCR (control, SEQ ID NO: 14) and the expression of mFAS is determined by FACS three days after the transfection.

FIG. 2D. Graph showing the fold change of the mFAS transcript extracted from the manipulated cells from (FIG. 2C), as measured by real-time qPCR. Expression of GUSB mRNA was used for qPCR normalization, and the graph was normalized to SSO SCR (control) transfected PBMCs.

FIG. 2E. Graph showing the fold change of FASΔEX6 transcript extracted from the manipulated cells (contacted with various SSO's) from (FIG. 2C) and measured as indicated in (FIG. 2D).

FIG. 2F. Graph showing the percentage of mFAS and FASΔEX6 transcripts out of the total FAS transcript in PBMCs from (FIG. 2C). percentage were calculated as in (FIG. 1D).

FIG. 2G. FACS analysis showing the percentage of mFAS positive cells in PBMCs transfected with SSO #6 (cells as described in FIG. 2C). Cells are gated on live singlet PBMCs. Results are presented as scatter plot (upper panels) or as histograms (lower panel).

FIG. 2I. Graph showing the fold change of mFAS (upper panel) and FASΔEX6 (lower panel) transcripts eight days following transfection with SSO #1 and SSO #6 as measured by Real-time qPCR as in (FIG. 2D, 2E).

FIG. 3A-3D. FAS Exon skipping induced by CRISPR/Cas9 splice site disruption, leading to ‘positive feedback’

FIG. 3A. Schematic representation of CRISPR/Cas9 system targeting the 3′SS of FAS exon 6. The target DNA sequence (coding strand) is denoted by SEQ ID NO: 7 (from 5′ to 3′), the sgRNA is denoted by SEQ ID NO: 16, and the sequence of the template is denoted by SEQ ID NO: 36.

FIG. 3B. FACS analysis of mFAS expression in wild type JURKAT cells and in JURKAT cells double transduced with lentivirus carrying Cas9 vector and with either lentivirus carrying CRISPR sgRNA targeting human FAS exon 3 (CRISPR_KO) or lentivirus targeting the FAS exon 6 3SS (CRISPR_AS, FASΔEX6).

The percentage of the cells expressing mFAS was determined by FACS at the indicated time points following transduction. Results are presented as scatter plot (upper panels) or as histograms (lower panels).

FIG. 3C. Graph showing mFAS (left panel) and FASΔEX6 (right panel) transcripts fold change extracted from 3SS CRISPR/Cas9 disrupted JURKAT cells from (FIG. 3B) and measured by Real-time qPCR.

FIG. 3D. Graph showing mFAS and FASΔEX6 transcripts as in (FIG. 3C) from wild type and 3SS CRISPR/Cas9 disrupted JURKAT cells, and total FAS transcript for each population. The total FAS transcript was calculated as in (FIG. 1D).

FIG. 4A-4F. In depth characterization of single cell FAS manipulated JURKAT cells

FIG. 4A. Graph of mFAS expression in single cell clone of JURKAT manipulated by FAS exon 6 skipping using CRISPR/Cas9 splice site disruption system, as analyzed by FACS. Single cell clones were generated by dividing the cells from (FIG. 3B) to 96 wells plate in a ratio of 0.1 cells/well. One representative graph is shown.

FIG. 4B. FAS DNA sequence in cells from (FIG. 4A). DNA extracted from SCR sgRNA transduced JURKAT single clone (SCR, as denoted by SEQ ID NO: 37) and 3SS CRISPR/Cas9 disrupted JURKAT cells (FASΔEX6) and the intron 5 and exon 6 junction was sequenced (as denoted by SEQ ID NO: 38).

FIG. 4C. FAS mRNA transcripts of cells shown in (FIG. 4B). mRNA was extracted and PCR and agarose gel electrophoresis were carried out using primers in exon 5 and exon 7 of FAS transcript. Shorter fragment obtained in FASΔEX6 single clone.

FIG. 4D. Sequence of representative FASΔEX6 JURKAT single clone mRNA (as denoted by SQ ID NO: 41) compared with SCR single clone (as denoted y SEQ ID NO: 40). The mRNA from (FIG. 4C) was sequenced, validating exon 6 skipping in the FASΔEX6 single clone.

FIG. 4E. Histogram showing FASΔEX6 Secretion as determined by ELISA from medium collected from manipulated FASΔEX6 and SCR JURKAT cells. Single clones from (FIG. 4A) were cultured for 24 h, and secretion of FASΔEX6 was analyzed by ELISA system.

FIG. 4F. Histogram showing the total fas transcript in FASΔEX6 JURKAT single clone compared with SCR. Real time qPCR using primers for exons 7 and 8 was carried out in cells from (FIG. 4B). Expression of GUSB mRNA was used for normalization.

FIG. 5A-5I. mFAS: FASΔEX6 ratio alteration, enhanced cytokine secretion, cell activation and survival of PBMCs through FASΔEX6 AS FIG. 5A. FACS analysis of healthy donor PBMCs activated as in (FIG. 2B) and transduced with lentivirus carrying CRISPR sgRNA targeting FAS exon 3 (CRISPR_KO). The percentage of transduced cells was determined by measuring the blue florescence protein (BFP) positive cells by FACS. One representative experiment is shown. Results are from triplicate.

FIG. 5B. FACS analysis of FAS positive cells in healthy donor's PBMCs activated, transduced with CRISPR_AS lentivirus, and electroporated with Cas9 protein. The percentage of FAS⁺ cells was determined by FACS five days following electroporation and compared to Wild Type PBMCs (non-manipulated cells). Results are presented as scatter plot (upper panel) or as a histogram (lower panel).

FIG. 5C. mFAS expression in transduced PBMCs before and after two repeated restimulations with plate bound aCD3 antibody. PBMCs as in (FIG. 5B) were restimulated twice with plate bound aCD3 antibody, 10 days after transduction and electroporation and then were stained and analyzed by flow cytometry for mFAS expression.

FIG. 5D. mFAS expression in transduced FASΔEX6 and SCR control cells at different time point. 6 days following transduction, FASΔEX6 PBMCs cell were restimulated and the mFAS expression was measured as in (FIG. 5B). The percentage of FAS negative cell population in FASΔEX6 PBMCs was increased from 16% (before stimulation, lower panel) to 34% following the second restimulation (upper panel).

FIG. 5E. IFNγ secretion as determined by ELISA from medium collected from three healthy donors restimulated FASΔEX6 transduced PBMCs (PBMCs as in 5B), restimulated FAS KO transduced PBMCs (CRISPR KO), and restimulated control SCR sgRNA transduced PBMCs (CRISPR_SCR). Medium was collected 48 h after restimulation and analyzed for IFNγ secretion by ELISA system. One representative donor is shown. Error bars represent s.d.

FIG. 5F. CD40L and 4-1BB expression in PBMCs transduced cells after restimulation with plate bound anti-CD3 antibody. PBMCs as in FIG. 5B were stained and analyzed by flow cytometry for CD40L and 4-1BB using FITC anti-human CD154 Antibody (BD 310804) and APC anti-human CD137 (4-1BB) Antibody (BD 309810).

FIG. 5G. FAS and CD25 expression in PBMCs transduced cells after restimulation with plate bound anti-CD3 antibody. PBMCs as in FIG. 5B were stained and analyzed by flow cytometry for FAS using Brilliant Violet 421™ anti-human CD95 (Fas) Antibody (BD 305624) and APC/Fire™ 750 anti-human CD25 Antibody (BD 302642).

FIG. 5H. 4-1BB and CD25 expression in transduced CD8+ T cells after restimulation with plate bound anti-CD3 antibody. PBMCs as in FIG. 5B were stained and analyzed by flow cytometry for 4-1BB using APC anti-human CD137 (4-1BB) Antibody (BD 309810) and APC/Fire™ 750 anti-human CD25 Antibody (BD 302642). PE anti-human CD8 (BD 980902) and Brilliant Violet 421™ anti-human CD95 (Fas) Antibody (BD 305624) were used for CD8 and FAS staining.

FIG. 5I. CD40L expression in transduced CD8− T cells after restimulation with plate bound anti-CD3 antibody. PBMCs as in FIG. 5B were stained and analyzed by flow cytometry for CD40L using FITC anti-human CD154 Antibody (BD 310804).

FIG. 5J. FACS analysis showing survival of melanoma target cells (mel624) co-cultured with PBMCs encoding NY-ESO1 TCR, that were manipulated with FASΔEX6, FAS KO or SCR. PBMCs were transduced as in (FIG. 5E). The manipulation rate for FAS KO and FASΔEX6 were 25%. The PBMCs were then transduced with retrovirus encoding NY-ESO1 TCR, that recognizes the NY-ESO antigen on the target mel624 cells. Transduced PBMCs were co-culture in one or two rounds with the target cells and CFSE in 1:1 effector:target (E:T) ratio for 24 hours, with an interval of 48 h. data is presented as scatter plot.

FIG. 5K. Graph showing the survival rates of target cells following one or two rounds of co-culturing with PBMCs manipulated FASΔEX6, FAS KO and SCR effector cells. The survival rates were calculated from the results obtained in (FIG. 5J) by the formula of: (Live mel624/Total live cells). Error bars represent s.d.

FIG. 6A-6H. Advantage of FASΔEX6NY manipulated PBMCs in pre-ACT context that includes rapid expansion

FIG. 6A. CD107 and IFNγ expression in CD8+ subpopulation from restimulated expanded FASΔEX6NY-ESO-TCR+ and SCR-ESO-TCR (as in FIG. 5J). PBMCs were transduced as in (FIG. 5H) and expanded by anti-CD3 and screened PBMCs as feeders for 12 days. The expanded cells were restimulated twice with 1 ug/ml anti-CD3 and anti-CD28 antibodies, with intervals of 48 h. Expression of intracellular IFNγ and CD107a was analyzed by flow cytometry after 6 h of activation.

FIG. 6B. IFNγ secretion as determined by ELISA from medium collected from restimulated expanded cells as in (FIG. 6A). Medium was collected 24 hr after the first and the second restimulation (left and right panels, respectively) and analyzed for IFNγ secretion by ELISA system. Error bars represent s.d.

FIG. 6C. GranzymeB secretion as determined by ELISA from medium collected from restimulated expanded cells as in (FIG. 6A). Medium was collected 24 hr after the first and the second restimulation and analyzed for GranzymeB secretion by ELISA system. Error bars represent s.d.

FIG. 6D. FACS histogram showing mFAS expression in restimulated expanded FASΔEX6NY-ESO-TCR+. Rapid expanded cells from (FIG. 6A) were analyzed for mFAS expression prior and following a second restimulation.

FIG. 6E. Apoptosis assay for restimulated expanded FASΔEX6NY-ESO-TCR+ and control SCR cells. Transduced and expanded cells as in (FIG. 6A) were restimulated with 1 ug/ml anti-CD3 and anti-CD28 antibodies for 24 h. Cells were analyzed for apoptosis with Annexin V (early apoptosis) and viability dye staining (late apoptosis).

FIG. 6F. Carboxyfluorescein Succinimidyl Ester (CFSE) proliferation assay for restimulated expanded FASΔEX6NY-ESO-TCR+(lower panel) and control SCR (upper panel) cells. Transduced and expanded cells as in (FIG. 6A) were stained with CFSE and restimulated as in (FIG. 6E). Cells were analyzed for proliferation 24 h and 96 h after the activation by flow cytometry.

FIG. 6G. CD107 and IFNγ expression in CD8+ subpopulation from expanded control SCR-ESO-TCR+ cells (upper panels) and FASΔEX6NY-ESO-TCR+ cells (lower panels) co-cultured with mel624 target cells in various effector:target ratios (E:T ratio). Cells as in (FIG. 6A) were restimulated with 1 ug/ml anti-CD3 and anti-CD28 antibodies for 24 h, and following additional 24 h co-cultured with smel624 in the indicated E:T ratios. Expression of intracellular IFNγ and CD107a was analyzed by flow cytometry as in (FIG. 6A).

FIG. 6H. Histogram showing the survival rates of melanoma target mel624 cells co-cultured with either FASΔEX6 PBMCs or SCR control cells. PBMCs were co-culture with mel624 as in (FIG. 6G) for 24 hours and analyzed by flow cytometry. Survival rates were calculated by the formula: (Live mel624/Total live cells). Error bars represent s.d.

FIG. 7A-7C. In vivo mouse model for evaluation of FASΔEX6 adoptive cell transfer-(ACT).

FIG. 7A. FACS analysis for mFAS expression in FASΔEX6 Pmel CD8+ T cells. CD8+ T cells were isolated from Pmel/Cas9 mouse splenocytes, activated with 2 ug/ml anti-mouse anti-CD3 and anti-CD28 antibodies, and transduced with sgRNA lentivirus for mouse FAS Exon 6 3′ SS disruption. mFAS expression was measured by FACS 10 days after transduction.

FIG. 7B. IFNγ secretion as determined by ELISA from medium collected from FASΔEX6 Pmel CD8+ T cells as in (FIG. 7A) or CD8+ mouse T cells transduced with sgRNA targeting mouse FAS exon 2 for FAS KO or SCR. Cells were co-cultured with the target cells B16-F10^{mhgp100} at the indicated E:T ratios. Medium was collected and analyzed for IFNγ secretion by ELISA system. Error bars represent s.d.

FIG. 7C. Graph showing the survival rates of target B16-F10^{mhgp100} cells co-cultured with either transduced FASΔEX6 Pmel CD8+ T cells, FAS KO and control SCR cells as in (FIG. 7B). PBMCs were co-culture with B16-F10^{mhgp100} as in (FIG. 6G) for 24 hours and analyzed by flow cytometry. Survival rates were calculated by the formula: (Live B16-F10^{mhgp100} Total live cells). Error bars represent s.d.

FIG. 8. Schematic timeline of human Adoptive Cell Transfer (ACT) protocol in melanoma-bearing murine

Schematic timeline of human Adoptive Cell Transfer (ACT) protocol in melanoma-bearing murine model. Human PBMCs are manipulated ex vivo with FAS-targeted gRNA lentivirus (FASΔEX6), co-transduced with NY-ESO-1 TCR retrovirus, and electroporated with recombinant Cas9 protein. Following FAS-based sorting, manipulated T cells undergo rapid expansion and are administered intravenously to A375-luciferase tumor-bearing NSG mice. IL-2 is administered post-transfer. On day 6 tumor-infiltrating lymphocytes (TILs) and splenocytes are harvested for analysis. Tumor size and survival are monitored longitudinally.

FIG. 9A-9B. Reduced tumor weights at day 6 post-Adoptive Cell Transfer (ACT) in melanoma-bearing NSG mice treated with FAS-targeted gRNA lentivirus (FASA EX6)

FIG. 9A: Representative images of tumors harvested from mice treated with FASΔEX6 T cells (SEQ ID NO: 15), a non-targeting sgRNA (SCR, SEQ ID NO: 23) or untreated melanoma-bearing NSG mice, showing visibly reduced tumor mass relative to SCR and untreated control groups.

FIG. 9B: Histogram depicting tumor weights at day 6 post-ACT across treatment groups. *p-value<0.05; **p-value<0.01; n=3 mice per group.

FIG. 10A-10B. Flow cytometry analysis of FAS and activation marker expression in tumor-infiltrating lymphocytes (TILs) isolated from tumors of melanoma-bearing NSG mice treated with FAS-manipulated T cells or controls

FIG. 10A: FACS analysis of FAS expression in TILs harvested 6 days after ACT with FASΔEX6 T cells (SEQ ID NO: 15) or control non-targeting sgRNA (SCR, SEQ ID NO: 23) T cells.

FIG. 10B: FACS analysis of the activation marker 4-1BB expression in TILs from tumors treated with FASΔEX6 or SCR T cells, measured 6 days after ACT.

FIGS. 11A-11C. Flow cytometry analysis of CD107a, IFN-γ, and TNF-α expression in splenic CD8+ T cells isolated from FASΔEX6- and SCR-treated mice upon ex vivo restimulation

FASΔEX6 and SCR T lymphocytes were isolated from splenic tissue of sacrificed mice at day 6 post-ACT and subjected to ex vivo stimulation with 1 μg/ml anti-CD3 and 1 μg/ml anti-CD28 antibodies for 6 hours. Marker expression was measured by flow cytometry.

FIG. 11A: Flow cytometry analysis of CD107a expression. Cells were stained using CD107 antibody (BL-328626) and intracellular CD107a expression was analyzed following 6 hours of stimulation.

FIG. 11B: Flow cytometry analysis of IFN-γ expression. Cells were stained using IFN-γ antibody (BL-502516) and intracellular IFN-γ expression was analyzed following 6 hours of stimulation.

FIG. 11C: Flow cytometry analysis of TNF-α expression. Cells were stained using TNF-α antibody (BL-502915) and intracellular TNF-α expression was analyzed following 6 hours of stimulation.

FIGS. 12A-12C. Longitudinal analysis of tumor growth and overall survival in melanoma-bearing NSG mice treated with FAS-targeted gRNA lentivirus (FASA EX6), non-targeting control sgRNA (SCR), or left untreated

FIG. 12A: Representative bioluminescence imaging of mice treated with FASΔEX6 T cells (SEQ ID NO: 15), SCR T cells (SEQ ID NO: 23), or untreated controls. Images acquired at day 0, 14, and 28 post-ACT.

FIG. 12B: Graph depicting median tumor volume at various time points post-ACT across treatment groups.

FIG. 12C: Kaplan-Meier survival curve showing survival rates at various time points post-ACT across treatment groups.

FIGS. 13A-13B. Apoptosis analysis in SCR and FASΔEX6 JURKAT clones following exposure to increasing concentrations of recombinant FAS ligand (0, 500, 1000, or 2500 ng/mL) for 24 hours

FIG. 13A Single-clone SCR and FASΔEX6 JURKAT cells were incubated with recombinant FAS ligand at the indicated concentrations for 24 hours. Apoptosis was assessed by flow cytometry using Annexin V (early apoptosis) and viability dye staining (late apoptosis). One representative experiment is shown.

FIG. 13B Graph showing the quantification of early and late apoptotic cell populations from flow cytometry analysis shown in FIG. 13A. Data represent mean values±standard deviation. Statistical comparisons were performed using an unpaired two-tailed Student's t-test. *P<0.05, **P<0.01, ***P<0.001.

FIG. 14. Evaluation of the protective effect of recombinant soluble FAS (sFAS) on SCR and FASΔEX6 JURKAT clones exposed to recombinant FAS ligand (500 ng/mL) Single-clone SCR and FASΔEX6 JURKAT cells were co-incubated with recombinant sFAS at ratios of 0:1, 2:1, and 20:1 (sFAS:FASL) for 24 hours. Apoptosis was assessed by flow cytometry using Annexin V (early apoptosis) and viability dye staining (late apoptosis). One representative experiment is shown. Apoptosis was assessed by flow cytometry using Annexin V (early apoptosis) and viability dye staining (late apoptosis). One representative experiment is shown. On the bottom, right side is a graph showing the quantification of early and late apoptotic cell populations from the flow cytometry analysis. Data represents mean values±standard deviation. Statistical comparisons were performed using an unpaired two-tailed Student's t-test. *P<0.05, **P<0.01, ***P<0.001.

FIG. 15. ELISA-based detection of free recombinant FASL in conditioned media from JURKAT clones following stimulation

Single-clone SCR, FAS-KO, and FASΔEX6 JURKAT cells were activated with 200 ng/mL PMA and 300 ng/mL ionomycin for 24 hours. Conditioned media were collected and incubated at 37° C. with increasing concentrations of recombinant FASL for one hour. Free (unbound) FASL was then measured using an ELISA competition assay. Reduced levels of free FASL in media from FASΔEX6 cells indicate binding by secreted soluble FAS. Data represents mean values±standard deviation. Statistical comparisons were performed using an unpaired two-tailed Student's t-test. *P<0.05, **P<0.01, ***P<0.001.

### DETAILED DESCRIPTION OF THE INVENTION

As shown by the present disclosure, manipulating FAS splicing and enhancing FAS exon 6 skipping combines the effect of membranal FAS (mFAS) knockdown together with soluble FAS (sFAS) overexpression. FAS knockdown has already shown promising effects in T cells. The effect of sFAS overexpression is less known, however, the results firstly presented by the present disclosure indicate the possibility that sFAS has a beneficial role as a decoy receptor for FASL. Furthermore, overexpression of sFAS may also supply environmental protection and block FAS-FASL interaction on bystander T cells. It can be also hypothesized that sFAS interaction with T cells membrane-bound FASL may lead to supportive reverse signaling, conducted by the FASL, which may promote immune cells' function and sustainability.

In the present disclosure, longitudinal qPCR for FAS alternative splicing isoforms after PBMCs activation showed an increase in membrane-bound form of FAS (mFAS) and a minor decrease in soluble form derived from exon 6 skipping (sFAS), turning the mFAS into the dominant isoform.

Using splice-switching oligonucleotides (SSOs) and CRISPR/Cas9 systems, the inventors were able to tilt FAS alternative splicing from the abundant membrane-bound form of FAS (mFAS) isoform to soluble form derived from exon 6 skipping (sFAS). It should be noted that throughout the present disclosure the term “sFAS” may be equivalently interchangeably replaced with the term “FASΔEX6”.

Accordingly, provided herein, are splicing manipulating agents that comprise nucleic acid molecules for FAS exon 6 skipping (SSO and sgRNA for CRISPR manipulation).

More specifically, CRISPR/Cas9 exon 6 splice site disruption method has another advantage in the form of positive feedback loop. The decrease in the mFAS transcript may cause upregulation of the FAS gene transcription, which can be spliced only to the sFAS form. Therefore, the signal for FAS transcription becomes constitutive, and there is an increase in positive feedback loop of sFAS transcript upregulation and production.

SSOs are short nucleic acid sequences that optionally, may comprise chemical modifications, designed to manipulate mRNA splicing by interrupting the linkage of splicing factors to regulatory motives in pre-mRNA sequences. SSO designed to target the 5′ splice-site (SS) manipulate FAS splicing led to high efficiency alternative splicing.

CRISPR/Cas9 system can also be used to target the 3′ SS of FAS exon 6, leading to double-strand breaking, non-homologous end joining, and disruption of the SS sequence. Therefore, the spliceosome is misidentifying the SS and skips over the exon. The total FAS transcript of CRISPR/Cas9 manipulated Jurkat cells was higher than the control population, apparently due to positive feedback loop: the decrease in the mFAS transcript caused upregulation of the FAS gene transcription, which can be spliced only to the sFAS form.

Taking advantage of FAS receptor alternative splicing manipulation may lead to better sustainability and functionality of immune cells in advanced cell-based therapies.

Provided herein are sequences for FAS exon 6 skipping (SSO and sgRNA for CRISPR manipulation). In addition, FAS manipulated T cells may be used for adoptive cell therapy. Thus, anew immunotherapy method is proposed using FAS manipulated T cells.

Indeed, using these systems, the alternative spliced population showed an increased secretion of IFNγ and GranzymeB following re-stimulation of the cells. Furthermore, following repeated re-stimulations, the alternative spliced population was positively selected naturally, had higher expression of activation markers, maintained the CD4+ and CD8+ sub-populations, as well as increased cell viability. Moreover, the alternative spliced population showed increased killing against target cells.

Accordingly, FAS receptor alternative splicing manipulation leads to better sustainability and functionality of immune cells. This approach can be used in immunotherapy, inter alia, in advanced cell-based therapies, and/or in any combination therapy approaches.

A first aspect of the present disclosure relates to a non-naïve cell of the T lineage manipulated, modulated and/or modified for enhanced skipping of exon 6 of the Fas (tumor necrosis factor (TNF) receptor superfamily, member 6) gene, or a cell population comprising at least one of the cell/s of the present disclosure. Specifically, the non-naïve manipulated cell of the T lineage in accordance with the present disclosure is manipulated and/or modified such that it predominantly expresses a soluble form of Fas (sFas, also referred to herein as FASΔEX6).

More specifically, a “non-naïve cell of the T lineage”, as used herein, refers to a specialized type of T cell that has undergone at least one of, prior activation, proliferation and/or differentiation to acquire specific functions, such that the cells are no longer considered naïve.

A “naïve T cell” in accordance with the present disclosure is a T cell that has differentiated in the thymus, and successfully undergone the positive and negative processes of central selection in the thymus. Among these are the naïve forms of helper T cells (CD4+) and cytotoxic T cells (CD8+). Any naïve T cell is considered immature and has not encountered its cognate antigen within the periphery. It should be understood that some T cells are incorporated within the naïve T cell phenotype but are a different T cell subset (Treg, regulatory T cells; RTE, Recent Thymic emigrant).

Still further, Naive T cells are commonly characterized by the surface expression of at least one of, the L-selectin (CD62L), and/or C-C Chemokine receptor type 7 (CCR7), and/or the CD45RA isoform (that may be also re-expressed on terminally exhausted TEMRA cells (terminally differentiated effector memory cells)). In yet some additional or alternative embodiments, naïve T cells may be characterized by the absence of the activation markers CD25, CD44 or CD69; and the absence of memory CD45RO isoform. Still further, in some embodiments, naïve cells may also express functional IL-7 receptors, that comprise subunits IL-7 receptor-α, CD127, and common-γ chain, CD132. In the naïve state, T cells are thought to require the common-gamma chain cytokines IL-7 and IL-15 for homeostatic survival mechanisms.

It should be understood that although CD62L, CCR7, and/or CD45RA, are expressed in naïve cells, they may also be expressed on early differentiated non naïve T cells. For example, Tscm, that are non-naïve cells encompassed by the present disclosure that express CCR7 and/or CD62L, and/or CD45RA, as well as other markers that characterize non-naïve cells. Thus, in some embodiments, non-naïve cells of the T lineage applicable in the present disclosure may also express CCR7 and/or CD62L. Still further, in some embodiments, non-naïve cells of the T lineage may comprise and/or express at least one of the following markers, specifically, activation markers such as CD25, CD44 and/or CD69; and/or the memory CD45RO isoform, and/or the CXCR3, CD11a, IL2Rb, CD58, CD57 markers.

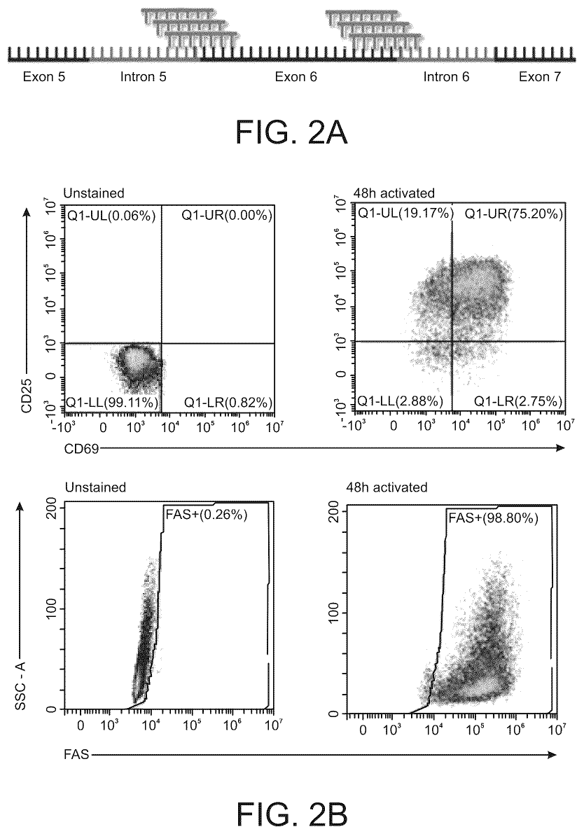

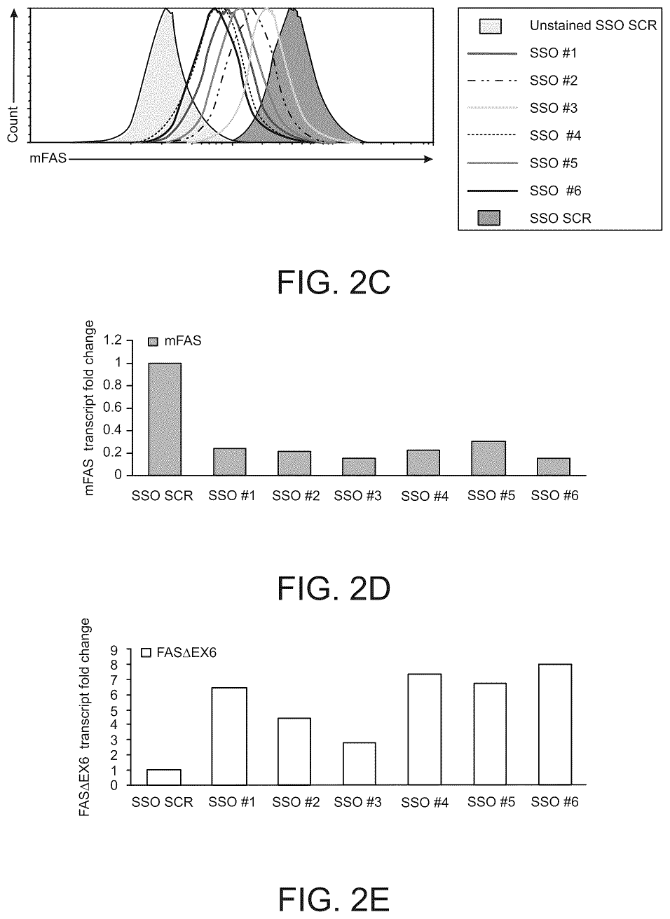

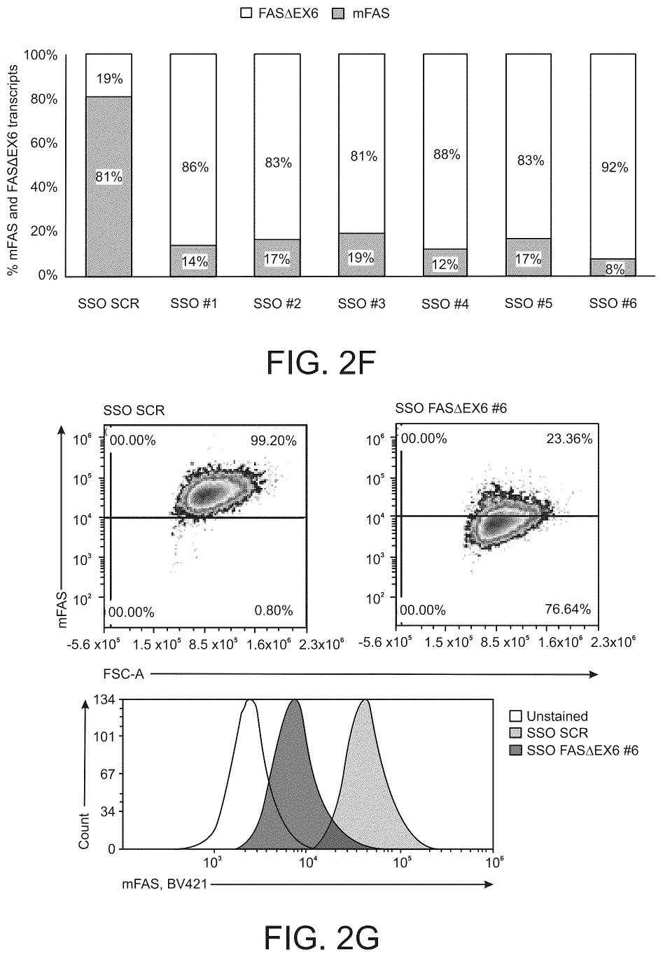

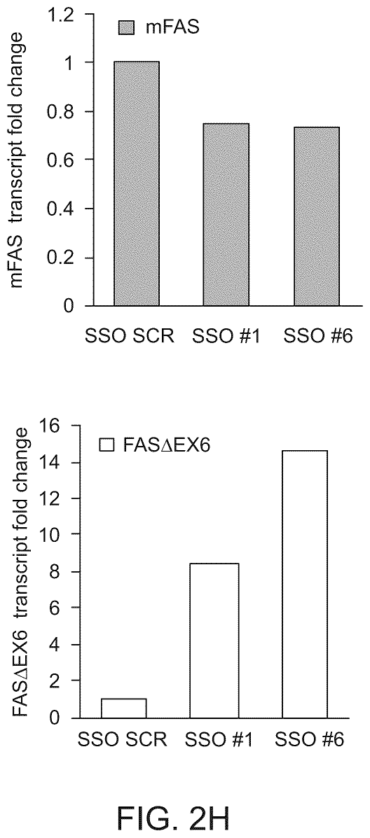

In yet some further embodiments, a non-naïve cell of the T lineage encompassed any activated cell of the T lineage. In some non-limiting embodiments, such activated cells express at least one of the characterizing markers disclosed herein above. In yet some further embodiments, the non-naïve cells applicable in the present disclosure may be activated by exposure to αCD3, and/or αCD28, and/or PMA and/or peptide antigen. Still further, as shown by FIG. 2B, T cells activated with anti-CD3 and anticD28 antibodies display expression of CD69 and/or CD25, that are markers for non-naïve cells. Thus, in some embodiments, the non-naïve cells of the present disclosure may express the CD69 and/or CD25 markers.

As indicted in the present aspect, the present invention provides a non-naïve cell of the T lineage that may be any lymphocyte, specifically, any lymphocyte of the T lineage. “Lymphocytes” are mononuclear nonphagocytic leukocytes found in the blood, lymph, and lymphoid tissues. They are divided on the basis of ontogeny and function into two classes, B and T lymphocytes, responsible for humoral and cellular immunity, respectively. Most are small lymphocytes 7-10 μm in diameter with a round or slightly indented heterochromatic nucleus that almost fills the entire cell and a thin rim of basophilic cytoplasm that contains few granules. When “activated” by contact with antigen, small lymphocytes begin macromolecular synthesis, the cytoplasm enlarges until the cells are 10-30 μm in diameter, and the nucleus becomes less completely heterochromatic; they are then referred to as large lymphocytes or lymphoblasts. These cells then proliferate and differentiate into B and T memory cells and into the various effector cell types: B cells into plasma cells and T cells into helper, cytotoxic, and suppressor cells.

Still further, in some embodiments, the manipulated cell of the present disclosure is a cell of the T lineage. A “T cell” or “T lymphocyte” as used herein is characterized by the presence of a T-cell receptor (TCR) on the cell surface. It should be noted that T-cells include helper T cells (“effector T cells” or “Th cells”), cytotoxic T cells (“Tc,” “CTL” or “killer T cell”), memory T cells, and regulatory T cells as well as Natural killer T cells, Mucosal associated invariants and Gamma delta T cells.

More specifically, Thymocytes are hematopoietic progenitor cells present in the thymus. Thymopoiesis is the process in the thymus by which thymocytes differentiate into mature T lymphocytes. The thymus provides an inductive environment, which allows for the development and selection of physiologically useful T cells. The processes of beta-selection, positive selection, and negative selection shape the population of thymocytes into a peripheral pool of T cells that are able to respond to foreign pathogens and are immunologically tolerant towards self-antigens.

Still further, in some embodiments of the present disclosure, the non-naïve cell of the T lineage manipulated in accordance with the present disclosure, is a T cell. For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, the spleen, lymph node, the thymus, or other tissues or fluids, for example, tumor tissue. T cells can also be enriched for or purified. The T cell may be a human T cell. The T cell may be a T cell isolated from a human. The T cell can be any type of T cell and can be of any developmental stage, provided that the cell is a non-naïve T cell, including but not limited to, CD4+/CD8+ double positive T cells, CD4⁺ helper T cells, e.g., Th¹ and Th² cells, CD8+ T cells (e.g., cytotoxic T cells), tumor infiltrating cells, memory T cells, Tscm (T memory stem (TSCM) cells), and the like. The T cell may be a CD8+ T cell or a CD4+ T cell.

In some embodiments of the present disclosure, the manipulated cell of the T lineage may be a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes. NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human).

Also provided by some embodiments of the present disclosure is a population of cells comprising at least one non-naïve cell of the T lineage manipulated for enhanced skipping of exon 6 of the Fas, specifically, the non-naïve cell of the T lineage described herein. The population of cells can be a heterogeneous population comprising the non-naïve cell of the T lineage manipulated for enhanced skipping of exon 6 of the Fas gene as described, in addition to at least one other cell, e.g., a host cell (e.g., a T cell), which was not manipulated for enhanced skipping of exon 6 of the Fas gene, or at least one cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly the non-naïve cell of the T lineage manipulated for enhanced skipping of exon 6 of the Fas gene (e.g., consisting essentially of). The population also can be a clonal population of cells, in which all cells of the population are clones of a single cell of the non-naïve cell of the T lineage manipulated for enhanced skipping of exon 6 of the Fas gene. In one embodiment of the present disclosure, the population of cells is a clonal population comprising cells that are non-naïve cell of the T lineage manipulated for enhanced skipping of exon 6 of the Fas gene as described herein. In some embodiments, the non-naïve cells of the T lineage manipulated for enhanced skipping of exon 6, form about 10% to about 100% of the cells in the cell population, specifically, about 10%, 20%, 30%, 40%, 50%, 60%, 70%, 89%, 90%, 100%.

Still further, the present disclosure provides cell manipulated for enhanced skipping of exon 6 of the FAS gene. The term “manipulation” and/or “manipulated” and/or “modulated” and/or “modified” and the like in the context of the subject disclosure refers to any deliberate alteration, modification exerted on cells, either in vitro or in vivo, to achieve desired enhanced skipping of exon 6 of the Fas gene. As explained in more detail below, this manipulation can involve various techniques, methodologies, or interventions aimed at modifying the alternative splicing of Fas gene, such as genetic modifications, epigenetic modifications, biochemical/chemical treatments, or any combination thereof. The purpose of manipulating cells in the context of the subject disclosure is to enhance skipping of exon 6 of the Fas (tumor necrosis factor (TNF) receptor superfamily, member 6) gene, thereby resulting in non-naïve manipulated cell of the T lineage which predominantly expresses a soluble form of Fas (sFas).

It should be understood that the manipulation may be either stable specifically affecting the genome of the cells and passing in any cell divisions and passages, or alternatively, transient. In transient manipulation it is meant that the modification involved are not stable and therefore are not maintained in the next generation, or even at a later stage in the same cell.

The term FAS gene, also known as TNFRSF6, FAS receptor or CD95, refers to the gene encoding the cell surface receptor protein FAS involved in regulating programmed cell death, a process known as apoptosis. The FAS gene is located on chromosome 10 (10q24.1) and consists of nine exons separated by eight introns. The boundaries of exon 2 to 5 encoding the extracellular region. Exon structure and functional protein domains correspond for exon 6 encoding the transmembrane region and for exon 9 encoding the “death domain” (Structure of the human APO-1 gene, European Journal of immunology, volume 24(12), 1994 pages 3057-3062).

The cells of the T lineage disclosed herein, are manipulated at the FAS gene. The FAS protein is a member of the tumor necrosis factor (TNF) receptor superfamily and plays a crucial role in controlling cell survival and immune response. The structure of the FAS gene allows for the production of a transmembrane receptor or a secreted protein which are involved in apoptotic and/or survival signaling. The alternative splicing of the FAS pre-mRNA can give rise to different isoforms of the FAS protein with varying functional properties. Dysregulation or mutations in the FAS gene can have implications for immune system function, autoimmune diseases, cancer, and other pathological conditions.

Therefore, provided by the present disclosure, is a non-naïve cell of the T lineage manipulated to enhanced skipping of exon 6 of the Fas, which encodes the transmembrane domain. This manipulation that specifically leads to enhanced skipping of exon 6, results in a significant reduction of the membranal form of FAS (mFAS). In yet some further embodiments, the manipulated cells of the T lineage display enhanced skipping of exon 6 that leads to a significant increase in the levels of the soluble form of FAS, thereby resulting in non-naïve manipulated cell of the T lineage which predominantly expresses a soluble form of Fas.

In some embodiments, Fas, as used herein refers to the human FAS. In some embodiments, the human Fas as used herein is encoded by the Fas gene that comprises the nucleic acid sequence as denoted by the NCBI Accession number. NC_000010.11. Still further, in some embodiments, the Fas gene comprises the nucleic acid sequence as denoted by SEQ ID NO: 31, or any homologs or variants thereof.

Still further, in some embodiments, the membranal FAS transcript, also referred to herein as the mFAS, may comprise in some embodiments, the nucleic acid sequence as denoted by NM_000043.6. Still further, in some embodiments, the mFAS transcript comprises the nucleic acid sequence as denoted by SEQ ID NO: 32, or any homologs or variants thereof. In some specific embodiments, the mFAS protein is as denoted by the NCBI accession number NP_000034.1. In some embodiments, the mFAS protein comprises the amino acid sequence as denoted by SEQ ID NO: 34, or any variants, mutants or homologs thereof.

In some embodiments, the soluble FAS transcript, that is also referred to herein as the soluble Fas. A “soluble protein” generally refers to a protein that is capable of being dissolved or dispersed in a liquid medium. Solubility is a characteristic that describes how well a substance (in this case, a protein) can dissolve in a particular solvent, such as water or another aqueous solution. It should be however noted that soluble for of Fas, in accordance with the present disclosure may further comprise any Fas variant that lacks the entire exon 6 or part of it. Still further, such Fas in some embodiments may not be connect/linked to the plasm membrane. sFAS may comprise the nucleic acid sequence as denoted by NM_152871.4. Still further, in some embodiments, the sFAS transcript comprises the nucleic acid sequence as denoted by SEQ ID NO: 33, or any mutants, homologs or variants thereof. In some embodiments, the sFAS transcript encodes the sFAS protein. In some specific embodiments, the sFAS protein is as denoted by the NCBI accession number NP_690610.1. In some embodiments, the sFAS protein comprises the amino acid sequence as denoted by SEQ ID NO: 35, or any variants, mutants or homologs thereof. It must be understood that in some embodiments, for example when a gene editing system is used for the manipulation of the cells of the T lineage towards enhanced skipping of exon 6, the resulting transcript may differ from the transcript disclosed above (SEQ ID NO: 33), at least in several nucleotides due to resulting mutations, such as indels, specifically, 1, 2, 3, 4, 5, 6, 7, 8, 9, 20 or more nucleotides differ. It should be noted that throughout the present disclosure, the term “sFAS” may be equivalently replaced with the term “FASΔEX6”, and the meaning of both terms similarly refer to soluble FAS as defined above.

Still further, the manipulated cell/s of the T linage in accordance with the present disclosure exhibit enhanced skipping of exon 6 of the FAS gene.

Still further, the manipulation of the cells of the T lineage results in cells displaying enhanced exon skipping. When referring to the term “enhanced”, it should be understood as increased, improved, intensified, augmented exclusion of exon 6 of the Fas gene as disclosed by the present disclosure, by any of the manipulation disclosed herein as compared to a suitable control, e.g., cells that are not manipulated for enhanced skipping of exon 6 of the Fas. Particularly, an enhanced skipping of exon 6 of the Fas gene can be an increase of about 5%, 10%, 15%, 20%, 25%, 30%, 35%, 40%, 45%, 50%, 55%, 60%, 65%, 70%, 75%, 80%, 85%, 90%, 95%, 99%, or more specifically 100%, as compared to exon skipping that occurs naturally with no manipulation as discussed herein.

In some embodiments, the non-naïve cell of the T lineage is manipulated by at least one splicing modulating agent or any vector, vehicle, matrix, nano- or micro-particle or composition comprising the at least one agent. Specifically, the splicing modulating agent comprising at least one nucleic acid molecule that targets at least one target nucleic acid sequence participating directly or indirectly in at least one splicing event of the Fas gene. In some embodiments, the introduction of the at least one agent into the cell induces at least one a splicing event via the target nucleic acid sequence, for example, a non-natural or aberrant splicing event that may not occurs in the absence of cell manipulation or may not occur in the extent demonstrated by the present disclosure in manipulated cells. In some embodiments, the introduction of the at least one splicing modulating agent into the cell induces at least one a splicing event via the target nucleic acid sequence, for example, enhancing a natural splicing event and shifting the balance toward a preferred variant compared to the non-manipulated cells as demonstrated by the present disclosure in manipulated cells.

In yet some further embodiments, the aberrant splicing event results in the predominantly expression of the soluble form of Fas.

When referring to the term “predominantly”, it is to be understood that the manipulated cells express mainly the soluble form of Fas, whether it is the mRNA transcript of soluble Fas or the soluble protein Fas. Still further, in accordance with the present disclosure at least 50%, 55%, 60%, 65%, 70%, 75%, 80%, 85%, 90%, 95%, 99.9% or more, specifically, 100% of the Fas mRNA transcript and/or the Fas protein expressed in the manipulated is the soluble form of Fas, which does not include exon 6.

Accordingly, in some embodiments, the splicing modulating agent provided herein modulate splicing of a target gene in order to generate aberrant or non-naturally occurring splicing events. Specifically, aberrant splicing event according to the disclosure relates to exon skipping. Such modulation includes promoting or inhibiting exon inclusion or exclusion. Still further, in some embodiments, aberrant splicing event results in mRNA transcripts comprised of a different combination of exons, specifically, excluding exon 6 of Fas. In certain embodiments, aberrant splicing event results in mRNA transcripts with deletions of exon 6. In certain embodiments, aberrant splicing event results in mRNA transcripts with deletions of portions of exon 6. In certain embodiments, aberrant splicing event results in mRNA transcripts comprising premature stop codons. Further provided herein are splicing modulating agents that comprise at least one nucleic acid molecule (e.g. antisense compounds such as oligonucleotides and gRNAs), that are targeted to target nucleic acid sequences that participate either directly or indirectly in the aberrant or induced splicing event. In some embodiments, the splicing modulating agents provided by the present disclosure target cis splicing regulatory elements present in pre-mRNA molecules, including exonic splicing enhancers, exonic splicing silencers, intronic splicing enhancers and intronic splicing silencers. Disruption of cis splicing regulatory elements is thought to alter splice site selection, which may lead to an alteration in the composition of splice products.

Alternative splicing or “splicing” as used herein, is the process by which exons of primary transcripts can be spliced into alternative arrangements to produce structurally and functionally different messenger RNA (mRNA) i.e., splicing variants. During splicing, introns are removed, and exons are joined together. the splicing process can be regulated by different cis- and trans-acting factors that influence the selection of specific splicing junctions. More specifically, splice junctions are also referred to as splice sites with the junction at the 5′ side of the intron often called the “5′ splice site,” or “splice donor site” and the junction at the 3′ side of the intron called the “3′ splice site” or “splice acceptor site.” In splicing, the 3′ end of an upstream exon is joined to the 5′ end of the downstream exon. Thus, the unspliced RNA (or pre-mRNA) has an exon/intron junction at the 5′ end of an intron and an intron/exon junction at the 3′ end of an intron. After the intron is removed, the exons are contiguous at what is sometimes referred to as the exon/exon junction or boundary in the mature mRNA. Point mutations in a gene may weaken or strengthen splice sites, enhancer or silencer elements or lead to their destruction. This in turn causes alteration of splicing events. More specifically, the exons to be retained in the mRNA are determined during the splicing process. The regulation and selection of splice sites are done by trans-acting splicing activator and splicing repressor proteins as well as cis-acting elements within the pre-mRNA itself such as exonic splicing enhancers and exonic splicing silencers.

The typical eukaryotic nuclear intron has consensus sequences defining important regions. Each intron has the sequence GU at its 5′ end. Near the 3′ end there is a branch site. The nucleotide at the branchpoint is always an A; the consensus around this sequence varies somewhat. The branch site is followed by a series of pyrimidines, the polypyrimidine tract then by AG at the 3′ end.

Splicing of mRNA is performed by an RNA and protein complex known as the spliceosome, containing snRNPs designated U1, U2, U4, U5, and U6 (U3 is not involved in mRNA splicing). U1 binds to the 5′ GU and U2, with the assistance of the U2AF protein factors, binds to the branchpoint A within the branch site. The complex at this stage is known as the spliceosome A complex. Formation of the A complex is usually the key step in determining the ends of the intron to be spliced out and defining the ends of the exon to be retained (the U nomenclature derives from their high uridine content). The U4, U5, U6 complex binds, and U6 replaces the U1 position. U1 and U4 leave. The remaining complex then performs two transesterification reactions. In the first transesterification, 5′ end of the intron is cleaved from the upstream exon and joined to the branch site A by a 2′,5′-phosphodiester linkage. In the second transesterification, the 3′ end of the intron is cleaved from the downstream exon, and the two exons are joined by a phosphodiester bond. The intron is then released in lariat form and degraded.

Splicing is regulated by trans-acting proteins (repressors and activators) and corresponding cis-acting regulatory sites (silencers and enhancers) on the pre-mRNA. However, as part of the complexity of alternative splicing, it is noted that the effects of a splicing factor are frequently position-dependent. That is, a splicing factor that serves as a splicing activator when bound to an intronic enhancer element may serve as a repressor when bound to its splicing element in the context of an exon, and vice versa. The secondary structure of the pre-mRNA transcript also plays a role in regulating splicing, such as by bringing together splicing elements or by masking a sequence that would otherwise serve as a binding element for a splicing factor. Together, these elements form a “splicing code” that governs how splicing will occur under different cellular conditions. There are two major types of cis-acting RNA sequence elements present in pre-mRNAs and they have corresponding trans-acting RNA-binding proteins. Splicing silencers are sites to which splicing repressor proteins bind, reducing the probability that a nearby site will be used as a splice junction. These can be located in the intron itself (intronic splicing silencers, ISS) or in a neighboring exon (exonic splicing silencers, ESS). They vary in sequence, as well as in the types of proteins that bind to them. The majority of splicing repressors are heterogeneous nuclear ribonucleoproteins (hnRNPs). Splicing enhancers are sites to which splicing activator proteins bind, increasing the probability that a nearby site will be used as a splice junction. These also may occur in the intron (intronic splicing enhancers, ISE) or exon (exonic splicing enhancers, ESE). Most of the activator proteins that bind to ISEs and ESEs are members of the SR protein family. Such proteins contain RNA recognition motifs and arginine and serine-rich (RS) domains.

In some embodiments, the aberrant splicing induced by the present disclosure may involve, and moreover, may enhance exon skipping, where an exon may be spliced out of the primary transcript or retained. More specifically, “Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered form of the protein. Still further, in some embodiments, alternative donor site, specifically, an alternative 5′ splice junction (donor site), changing the 3′ boundary of the upstream exon, and/or an alternative acceptor site, specifically, an alternative 3′ splice junction (acceptor site), changing the 5′ boundary of the downstream exon, may be utilized in exon skipping. As indicated above, the cell of the T lineage in accordance with the present disclosure was manipulated for exon skipping of exon 6 of the Fas gene.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. An “intron” refers to a nucleic acid region (within a gene) that is not translated into a protein. An intron is a non-coding section that is transcribed into a precursor mRNA (pre-mRNA), and subsequently removed by splicing during formation of the mature RNA.

A “Gene” as used herein in connection with the FAS gene, may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. Genes are composed of coding and non-coding transcripts, that may utilize the same sequence space on the same and opposite strands often each controlled by their own distinct regulatory regions. In general, “transcript” as used herein may refer to any nucleic acid or its sequence of any gene or gene combination and any variant thereof, in particular mRNA or cDNA sequence variants thereof. “Isoform” is used to relate to a particular variant of a transcript.

The disclosure involves the use of splicing modulating agent/s, or splicing modulator/s. “Modulator”, as used herein means a compound that leads or causes directly or indirectly to a perturbation of function or activity, specifically, of splicing. In certain embodiments, modulation means an increase, a decrease or alteration in splicing of a specific target. In some specific embodiments, modulators of splicing in accordance with the disclosure lead to aberrant splicing that in some embodiments results in skipping exon 6 of Fas.

As indicated above, the splicing modulating agent in accordance with the present disclosure may comprise a nucleic acid-based molecule. In some embodiments, the at least one nucleic acid sequence comprised within the splicing modulating agents used by the methods, manipulated cells, compositions of the disclosure, target at least one target nucleic acid sequence that participates directly or indirectly in at least one splicing event of the Fas gene. In some embodiments, such target nucleic acid sequence comprises at least one of, a splicing junction, a splice donor site, a splice acceptor site, a branch site, an exonic splicing enhancer, splicing silencer, an intronic splicing enhancer and an intronic splicing silencer of said target gene, specifically, the Fas gene, as specified above, more specifically exon 6 of Fas gene.

Thus, the target sequence for an aberrant splicing event may include any sequence within an exon, or within at least one intron located upstream or downstream to said exon, or within at least one splicing junction flanking said exon. More specifically, the target sequence may include any sequence comprised within a sequence flanking the 5′ end of an exon in a distance from about 1 to about 500 base pairs upstream of the indicated exon, or alternatively or additionally, any sequence comprised within a sequence flanking the 3′ end of an exon in a distance from about 1 to about 500 base pairs downstream of the indicated exon in a preprocessed target transcript. Specifically about 1, 5, 10, 15, 20, 25, 30, 35, 40, 45, 50, 55, 60, 65, 70, 75, 80, 85, 90, 95, 100, 150, 200, 250, 300, 350, 400, 450 or 500 base pairs downstream or upstream of the indicated exon. The term “flanked”, “flanking”, “flank”, as used herein refers to a nucleic acid sequence positioned between two defined regions. For example, the target exon is flanked by an intron/exon splice junction and an exon/intron splice junction, where the intron/exon splice junction is positioned 5′ (or upstream) to the exon and the exon/intron splice junction is positioned 3′ (or downstream) to the exon.

The nucleic acid sequence of the splicing modulating agent of the methods of the disclosure targets a target nucleic acid. As used herein, “target nucleic acid” means a nucleic acid molecule to which an antisense compound (e.g., oligonucleotide or complementary guide RNAs) hybridizes. As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions, as will be further detailed herein after.

Accordingly, in some embodiments, the target nucleic acid sequence for an aberrant splicing event, specifically, skipping of exon 6, is comprised within and/or comprises at least one of: 5′ splice site (5′SS) of exon 6, 3′ splice site (3′SS) of exon 6, exon 6, at least one intron located upstream and/or downstream to said exon 6, and/or at least one splicing junction flanking exon 6 of Fas gene.

In some embodiments, the target nucleic acid sequence for an aberrant splicing event that manipulates and/or modifies the disclosed cells, is comprised within intron 5 and exon 6 of the Fas gene. In some other embodiments the target nucleic acid sequence is comprised within exon 6 and intron 6 of the Fas gene. In some other specific embodiments, the target nucleic acid sequence for an aberrant splicing event is comprised within and/or comprises the 3′ splice site (3′SS) of exon 6.

In some embodiments the splicing modulating agent used in the cells/compositions/kits or by the methods of the disclosure comprises at least one of an oligonucleotide and/or a compound of a gene editing system: In some embodiments, (a), at least one oligonucleotide which comprises a nucleic acid sequence complementary to at least part of the target nucleic acid sequence. In yet some further additional or alternative embodiments (b), the slicing modulating agent may comprise at least one nucleic acid sequence comprising at least one guide RNA (gRNA) that targets at least one protospacer within the target nucleic acid sequence, or any nucleic acid sequence encoding the gRNA. It should be understood that the gRNA guiding at least one programmable engineered nuclease (PEN) to the target nucleic acid sequence.

In some embodiments the splicing modulating agent used for manipulating the cells, in the compositions of the present disclosure and by the methods disclosed herein, may comprise at least one oligonucleotide. In more specific embodiments such oligonucleotide is an antisense oligonucleotide (ASO).

Thus, the present disclosure, as well as the cells, methods, compositions and splicing modulators disclosed herein provide oligonucleotides for modulating splicing events, thereby leading to skipping of exon 6 of Fas. As used herein, “oligonucleotide” means a compound comprising a plurality of linked nucleosides. More specifically, single strand or double strand oligomer or polymer of ribonucleic acid molecules, deoxyribonucleic acid molecules and any combinations thereof. In certain embodiments, an oligonucleotide comprises one or more unmodified ribonucleosides (RNA) and/or unmodified deoxyribonucleosides (DNA) and/or one or more modified RNA and/or modified DNA.

As used herein, “modified oligonucleotide” means an oligonucleotide comprising at least one modified nucleoside and/or at least one modified internucleoside linkage. More specifically, “Nucleobase” means the heterocyclic base portion of a nucleoside. Nucleobases may be naturally occurring or may be modified. In certain embodiments, a nucleobase may comprise any atom or group of atoms capable of hydrogen bonding to a nucleobase of another nucleic acid. “Nucleotide” means a nucleoside comprising a phosphate linking group. As used herein, nucleosides include nucleotides. “Modified nucleoside” a nucleoside comprising at least one modification compared to naturally occurring RNA or DNA nucleosides. Such modification may be at the sugar moiety and/or at the nucleobase. “Oligonucleoside” means an oligonucleotide in which none of the internucleoside linkages contains a phosphorus atom. As used herein, oligonucleotides include oligonucleosides. “Modified oligonucleotide” means an oligonucleotide comprising at least one modified nucleoside and/or at least one modified internucleoside linkage.

In some specific embodiments, the oligonucleotides disclosed by the present disclosure may be antisense oligonucleotides, specifically, AON/s, or ASO (antisense oligo), and Splice-Switching Oligonucleotides (SSOs), that are used herein interchangeably. Provided herein are antisense compounds useful for modulating RNA splicing via antisense mechanisms of action, including antisense mechanisms based on target occupancy. More specifically, an “antisense” is a single strand nucleic acid molecule that is complementary to one of the strands of a target nucleic acid molecule of a specific target gene. Antisense sequence may inhibit or interfere with a splicing event by base-pairing to it and physically obstructing the splicing machinery. As used herein, “nucleobase complementarity” or “complementarity” when in reference to nucleobases means a nucleobase that is capable of base pairing with another nucleobase. For example, in DNA, adenine (A) is complementary to thymine (T), and cytosine (C) is complementary to Guanine (G). In RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity. Still further, as used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. In contrast, “non-complementary” in reference to nucleobases means a pair of nucleobases that do not form hydrogen bonds with one another. Complementary oligomeric compounds need not have nucleobase complementarity at each nucleoside. Rather, some mismatches are tolerated. Complementarity may be “partial,” in which only some of the nucleic acids' bases are matched according to base pairing rules. In some embodiments, there may be “complete” or “total” complementarity between the nucleic acids. The degree of complementarity between nucleic acid strands has significant effects on the efficiency and strength of hybridization between nucleic acid strands. In some embodiments, the complementarity may range between about 50% to about 100%. In certain embodiments, complementary oligomeric compounds or regions are complementary at 70% of the nucleobases to the target sequence (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary. While perfect complementarity is often desired, some embodiments can include one or more but preferably 6, 5, 4, 3, 2, or 1 mismatch/s with respect to the target nucleic acid sequence. Variations at any location within the oligomer are included. In certain embodiments, variations in sequence near the termini of an oligomer are generally preferable to variations in the interior, and if present are typically within about 6, 5, 4, 3, 2, or 1 nucleotide/s of the 5′ and/or 3′ terminus. In yet some further embodiments, the antisense oligomer of the disclosure may be said to be “directed to” or “targeted against” a target sequence with which it hybridizes. As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases. As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site. As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

Still further, “Antisense oligonucleotide” (SSO, or ASO/s as used herein interchangeably) means an oligomeric compound, at least a portion of which is at least partially complementary to a target nucleic acid to which it hybridizes, wherein such hybridization results in at least one antisense activity. In some specific embodiments, the ASOs of the disclosure are Splice-Switching Oligonucleotides (SSOs). In certain embodiments, the present disclosure provides antisense oligonucleotides of any of a variety of ranges of lengths. In certain embodiments, the present disclosure provides oligomeric compounds including oligonucleotides of any of a variety of ranges of lengths.

In certain embodiments, the disclosure provides oligomeric compounds or oligonucleotides consisting of X to Y linked nucleosides or nucleotides, where X represents the fewest number of nucleosides in the range and Y represents the largest number of nucleosides in the range. In certain such embodiments, X and Y are each independently selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 and more nucleosides or nucleotides; provided that X<Y. For example, in certain embodiments, the disclosure provides antisense compounds or antisense oligonucleotides comprising or consisting of: 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50 and more, specifically, 2-100 and more, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50 and more, specifically, 3-100 and more, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33, 4-34, 4-35, 4-36, 4-37, 4-38, 4-39, 4-40, 4-41, 4-42, 4-43, 4-44, 4-45, 4-46, 4-47, 4-48, 4-49, 4-50 and more, specifically, 4-100 and more, 5-9, 5-10, 5-11, 5-12, 5-13, 5-14, 5-15, 5-16, 5-17, 5-18, 5-19, 5-20, 5-21, 5-22, 5-23, 5-24, 5-25, 5-26, 5-27, 5-28, 5-29, 5-30, 5-31, 5-32, 5-33, 5-34, 5-35, 5-36, 5-37, 5-38, 5-39, 5-40, 5-41, 5-42, 5-43, 5-44, 5-45, 5-46, 5-47, 5-48, 5-49, 5-50 and more, specifically, 5-100 and more, 6-9, 6-10, 6-11, 6-12, 6-13, 6-14, 6-15, 6-16, 6-17, 6-18, 6-19, 6-20, 6-21, 6-22, 6-23, 6-24, 6-25, 6-26, 6-27, 6-28, 6-29, 6-30, 6-31, 6-32, 6-33, 6-34, 6-35, 6-36, 6-37, 6-38, 6-39, 6-40, 6-41, 6-42, 6-43, 6-44, 6-45, 6-46, 6-47, 6-48, 6-49, 6-50 and more, specifically, 6-100 and more, 7-9, 7-10, 7-11, 7-12, 7-13, 7-14, 7-15, 7-16, 7-17, 7-18, 7-19, 7-20, 7-21, 7-22, 7-23, 7-24, 7-25, 7-26, 7-27, 7-28, 7-29, 7-30, 7-31, 7-32, 7-33, 7-34, 7-35, 7-36, 7-37, 7-38, 7-39, 7-40, 7-41, 7-42, 7-43, 7-44, 7-45, 7-46, 7-47, 7-48, 7-49, 7-50 and more nucleosides or nucleotides, specifically, 7-100 and more, 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 8-31, 8-32, 8-33, 8-34, 8-35, 8-36, 8-37, 8-38, 8-39, 8-40, 8-41, 8-42, 8-43, 8-44, 8-45, 8-46, 8-47, 8-48, 8-49, 8-50 and more, specifically, 8-100 and more, 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 9-31, 9-32, 9-33, 9-34, 9-35, 9-36, 9-37, 9-38, 9-39, 9-40, 9-41, 9-42, 9-43, 9-44, 9-45, 9-46, 9-47, 9-48, 9-49, 9-50 and more, specifically, 9-100 and more, 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 10-31, 10-32, 10-33, 10-34, 10-35, 10-36, 10-37, 10-38, 10-39, 10-40, 10-41, 10-42, 10-43, 10-44, 10-45, 10-46, 10-47, 10-48, 10-49, 10-50 and more, specifically, 10-100 and more, nucleosides or nucleotides.

In some specific embodiments of the disclosure, oligomers for use in antisense applications for manipulating and/or modifying the disclosed cells of the T lineage, generally range in length from about 10 to about 50 bases, also referred to herein as subunits, more preferably about 10 to 30 bases/subunits, and typically 15-25 bases. For example, an oligomer of the disclosure having 15-20 bases/subunits, specifically, 15, 16, 17, 18, 19, 20, or more bases. In some embodiments the oligonucleotide used by the method of the disclosure may comprise at least 10 or more contiguous nucleobases complementary to at least part of the at least one nucleic acid sequence that participates directly or indirectly in at least one splicing event. In some embodiments, the splicing event discussed herein is in the Fas gene, specifically, a splicing event that results in skipping of exon 6 of Fas gene.

Specifically, at least 10 or more, at least 11 or more, at least 12 or more, at least 13 or more, at least 14 or more, at least 15 or more, at least 16 or more, at least 17 or more, at least 18 or more, at least 19 or more, at least 20 or more, at least 21 or more, at least 22 or more, at least 23 or more, at least 24 or more, at least 25 or more, at least 26 or more, at least 27 or more, at least 28 or more, at least 29 or more, at least 30 or more contiguous nucleobases. In yet some further specific embodiments, the oligonucleotide used by the methods of the disclosure may comprise at least fifteen contiguous nucleobases complementary to at least part of the at least one nucleic acid sequence that participates directly or indirectly in at least one splicing event. In some embodiments, the splicing event discussed herein is in the Fas gene, specifically, a splicing event that results in skipping of exon 6 of Fas gene.

Thus, in some embodiments, the splicing modulating agent used in the cells/compositions/kits or by the methods of the disclosure comprises at least one antisense oligonucleotide (ASO) and/or a splice switching antisense oligonucleotide (SSO). Specifically, the ASO and/or SSO comprises at least fifteen contiguous nucleobases complementary, or that display at least partial complementarity to at least part of the at least one nucleic acid sequence that participates directly or indirectly in at least one splicing event, specifically, at least one splicing event in the Fas gene.

In some other embodiments the SSO and/or ASO comprise nucleic acid sequence complementary to intron 5/exon 6 of the Fas gene. Specifically, complementarity to the sequence spanning from the end of intron 5, and the beginning of exon 6 of the Fas gene, or in other words, in the intron 5/exon 6 junction. In yet some further embodiments, the SSOs and/or ASOs of the present disclosure display complementarity to the sequence spanning from the end of exon 6 to the beginning of intron 6, or in other words, in the exon 6/intron 6 junction sequence. In other embodiments, the SSO and/or ASO comprising nucleic acid sequence complementary to exon 6/intron 6 of the Fas gene. In yet some other embodiments, the SSO and/or ASO comprising nucleic acid sequence complementary to both intron 5/exon 6 and exon 6/intron 6 of the Fas gene. In some embodiments, the SSO and/or ASO comprising nucleic acid sequence complementary to the 3′ and/or 5′ splice site of exon 6. In some embodiments, the SSO and/or ASO comprise nucleic acid sequence complementary to the 3′ splice site of exon 6. In some embodiments, the SSO and/or ASO comprise nucleic acid sequence complementary or complementary in part to the sequence as denoted by SEQ ID NO. 1, or any homologs or variants thereof (also referred to herein as the target site).

In some embodiments, the SSO used in the compositions/kits or by the methods of the disclosure may comprise the nucleic acid sequence as denoted by SEQ ID NO: 2, or any variants and/or derivatives thereof, and its target sequence in FAS is as denoted by SEQ ID NO: 3 (targeting 3′ splice site as denoted by SEQ ID NO. 1).

In some embodiments, the SSO used in the compositions/kits or by the methods of the disclosure may comprise the nucleic acid sequence as denoted by SEQ ID NO: 4 or any variants and/or derivatives thereof. In yet some further embodiments, such SSO targets a target sequence in FAS that comprises a sequence as denoted by SEQ ID NO: 5 (targeting 3′ splice site as denoted by SEQ ID NO. 1).

In yet some other embodiments, the SSO used in the compositions/kits or by the methods of the disclosure may comprise the nucleic acid sequence as denoted by SEQ ID NO: 6 or any variants and/or derivatives thereof. In yet some further embodiments, such SSO targets a target sequence in FAS that may comprise a sequence as denoted by SEQ ID NO: 7 (targeting 3′ splice site as denoted by SEQ ID NO. 1).

In yet some other embodiments, the SSO used in the compositions/kits or by the methods of the disclosure may comprise the nucleic acid sequence as denoted by SEQ ID NO: 8 or any variants and/or derivatives thereof. In yet some further embodiments, such SSO targets a target sequence in FAS, that may comprise a sequence as denoted by SEQ ID NO: 9 (targeting 5′ splice site as denoted by SEQ ID NO. 1).

In yet some other embodiments, the SSO used in the compositions/kits or by the methods of the disclosure may comprise the nucleic acid sequence as denoted by SEQ ID NO: 10 or any variants and/or derivatives thereof. In yet some further embodiments, such SSO targets a target sequence in FAS that comprises a sequence as denoted by SEQ ID NO: 11 (targeting 5′ splice site as denoted by SEQ ID NO. 1).

In yet some other embodiments, the SSO used in the compositions/kits or by the methods of the disclosure may comprise the nucleic acid sequence as denoted by SEQ ID NO: 12 or any variants and/or derivatives thereof. Still further, in some embodiments, such SSO targets a target sequence in FAS that may comprise a sequence as denoted by SEQ ID NO: 13 (targeting 5′ splice site as denoted by SEQ ID NO. 1).

The present disclosure also encompasses any homologues or variant of the SSOs and/or AONs of the present disclosure, specifically, those defined by their nucleic acid sequence according to the disclosure. The term “homologues” is used to define nucleic acid sequences (oligonucleotide) which maintain a minimal homology to the nucleic acid sequences defined by the disclosure, e.g. preferably have at least about 65%, more preferably at least about 70%, at least about 75%, even more preferably at least about 80%, at least about 85%, most preferably at least about 90%, at least about 95% overall sequence homology, specifically, with the entire nucleic acid sequence of any of the oligonucleotides as structurally defined above, e.g. of a specified sequence, more specifically, the nucleic acid sequence of the SSOs as denoted by any one of SEQ ID NOs. 2, 4, 6, 8, 10 and 12, and any variants and derivatives thereof. The term “derivative” or “variant” is used to define nucleic acid sequences (oligonucleotide), with any insertions, deletions, substitutions and modifications of between about 1 to 10 bases, to the nucleic acid sequences that do not alter the activity of the original SSOs (specifically, to induce aberrant splicing event in the target sequence). It should be understood that all SSOs modifications disclosed by the disclosure in connection with other aspects of the disclosure, are also applicable in the present aspect.

In some embodiments, the oligonucleotides provided and used by the disclosure may comprise DNA, RNA, any derivatives thereof or any combinations thereof. More specifically, the currently used antisense oligonucleotides are rarely regular RNA or DNA oligonucleotide, as alternative antisense oligonucleotide chemistries have been developed to improve affinity, boost stability in the circulation and in target cells, and enhance cell penetration and nuclear accumulation. The non-bridging oxygen in the phosphate backbone may be replaced with a sulfur atom, generating phosphorothioate (PS) AONs. This modification enhances cellular uptake and improves resistance to nucleases but reduces the affinity of the AON to the target RNA. Addition of a methyl or a methoxyethyl group to the 2′-O atom of the ribose sugar (2′OMe and 2′OMOE, respectively) renders the AON-target RNA hybrid RNase H-resistant and increases the affinity of the AON for the target RNA. Most AONs have both the 2′O and the phosphorothioate (PS) modification (2′OMe-PS and 2′OMOE-PS) since they have a good safety-profile, and their synthesis is relatively inexpensive. “2′-OMe” or “2′-OCH₃” or “2′-O-methyl” each means a nucleoside comprising a sugar comprising an —OCH₃ group at the 2′ position of the sugar ring. “MOE” or “2′-MOE” or “2′-OCH₂CH₂OCH₃” or “2′-O-methoxyethyl” each means a nucleoside comprising a sugar comprising a —OCH₂CH₂OCH₃ group at the 2′ position of the sugar ring.

In a different available oligonucleotide chemistry, a methylene bridge connects the 2′-O and the 4′-C of the ribose, forcing the nucleotide in the “endo” conformation, in what has been dubbed “locked nucleic acid” (LNA). This modification leads to a very high affinity for the target nucleic acid. In addition to the described negatively charged oligonucleotides (2′OMe-PS, 2′OMOE-PS, and LNA), two more oligonucleotide chemistries may be used in attempts to modulate splicing in accordance with some embodiments of the disclosure, specifically, peptide nucleic acids (PNAs) and phosphorodiamidate morpholino oligomers (PMOs). Both these types of charge-neutral oligonucleotides are resistant to exo- and endonucleases and RNase H cleavage. PNAs have a 2-aminoethyl glycine backbone linked to nucleobases and show high affinity to both RNA and DNA targets and good sequence specificity. PMOs consist of morpholine rings that are connected through phosphorodiamidate groups. The terms “morpholino oligomer” or “PMO” (phosphoramidate- or phosphorodiamidate morpholino oligomer) refer to an oligonucleotide analog composed of morpholino subunit structures, where (i) the structures are linked together by phosphorus-containing linkages, one to three atoms long, preferably two atoms long, and preferably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide. Variations can be made to this linkage as long as they do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to phosphorus may be unsubstituted, monosubstituted, or disubstituted with (optionally substituted) lower alkyl. The purine or pyrimidine base pairing moiety is typically adenine, cytosine, guanine, uracil, thymine or inosine. The synthesis, structures, and binding characteristics of morpholino oligomers are detailed in U.S. Pat. Nos. 5,698,685, 5,217,866, 5,142,047, 5,034,506, 5,166,315, 5,521,063, 5,506,337, 8,076,476, 8,299,206 and 7,943,762 (cationic linkages). It should be understood that the disclosure further encompasses any oligonucleotide modification known in the art, specifically, any one of LNOs, PMOs, 2′-O-ME, 2′-MOE, 2′-Flour, and the like. In certain embodiments, the antisense oligonucleotides of the present disclosure are modified by attachment of one or more conjugate groups. In general, conjugate groups modify one or more properties of the attached oligomeric compound including but not limited to, pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety, cholic acid, a thioether, e.g., hexyl-S-tritylthiol, a thiocholesterol, an aliphatic chain, e.g., do-decan-diol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

In some embodiments, antisense oligonucleotides/AONs include both GAPmers that induce degradation of mRNAs (e.g., RNase H cleavage) and Splice-Switching Oligonucleotides (SSOs) known in the art. It should be understood that the disclosure relates to any SSOs. More specifically, in some embodiments, the ASO of the disclosure does not include GAPmers. In addition to modulation of the splicing events by ASOs of the disclosure, one can use a different approach where the endogenous Double-stranded RNA-specific adenosine deaminase (ADAR) RNA editing enzymes are recruited and guided to edit a selected target to change base at key splicing site. This could be achieved by introducing an antisense oligonucleotide that, when bound to its target, assumes a dsRNA configuration similar to that of a typical ADAR editing site and instigates editing. Thus, in some embodiments, the ASOs used and/or provided by the disclosure may be suitable for guiding and recruiting ADAR proteins, thereby leading to aberrant splicing event in the target sequence.

In certain embodiments, the splicing modulating agents, specifically, oligonucleotides comprise one or more terminal stabilizing group that enhances properties such as for example nuclease stability. Included in stabilizing groups are cap structures. The terms “cap structure” or “terminal cap moiety,” as used herein, refer to chemical modifications, which can be attached to one or both of the termini of an oligomeric compound. Certain such terminal modifications protect the oligomeric compounds having terminal nucleic acid moieties from exonuclease degradation and can help in delivery and/or localization within a cell. The cap can be present at the 5′-terminus (5′-cap) or at the 3-terminus (3′-cap) or can be present on both termini.

Still further, in some embodiments, the antisense compounds of the disclosure may include an oligonucleotide moiety conjugated to a CPP, preferably an arginine-rich peptide transport moiety effective to enhance transport of the compound into cells. The transport moiety is preferably attached to a terminus of the oligomer. In some embodiments, the cell-penetrating peptide may be an arginine-rich peptide transporter. In other embodiments, the cell-penetrating peptide may be Penetratin or the Tat peptide. These peptides are well known in the art.

Still further, it should be noted that physical methods applied for in vitro and in vivo ASOs delivery are based on making transient penetration in cell membrane by mechanical, electrical, ultrasonic, hydrodynamic, or laser-based energy so that DNA entrance into the targeted cells is facilitated.

As described above, the ASO and/or SSO used in the cells/compositions/kits or by the methods of the disclosure are chemically modified. It should be appreciated that the ASO and/or SSO used for manipulating the disclosed non-naïve cells of the T lineage may comprise any of the modifications disclosed herein above.

In yet some more specific embodiments, the SSO may comprise a phosphorothiate (PS) modifications. It is well known that ASO that are fully modified at the 2′ sugar position confer RNAs H resistance and may be used as SSO. In some specific embodiments, the SSO used in the compositions/kits or by the methods of the disclosure are modified at the 2′ position and comprises a 2′-O-methoxyethyl modification. In some other embodiments, the SSO used in the compositions/kits or by the methods of the disclosure are modified at the 2′ position and comprises a 2′-O-methyl modification (2′-OME).

In yet some further additional or alternative embodiments, the skipping of exon 6 of the Fas gene may be performed or facilitated using a gene editing system. Thus, as indicated above, in some alternative or additional embodiments, the splicing modulating agent used in the cells/compositions/kits or by the methods of the disclosure comprises at least one nucleic acid sequence comprising at least one component of a gene editing system designated to participate in a gene editing even that leads to skipping of exon 6 of the Fas gene. In some embodiments, the at least one component of a gene editing system may comprise at least one guide RNA (gRNA) that targets at least one protospacer within the target nucleic acid sequence, or any nucleic acid sequence encoding the gRNA. Specifically, the gRNA guides at least one programmable engineered nuclease (PEN) to the target nucleic acid sequence.

In yet some alternative embodiments the splicing modulating agent may be at least one guide RNA that guides at least one PEN to the at least one target nucleic acid sequence as specified herein. In some embodiments, the PEN comprises at least one clustered regulatory interspaced short palindromic repeat (CRISPR)/CRISPR associated (cas) protein.

Thus, according to some embodiments, the splicing modulating agent of the present disclosure, used in the cells, methods, compositions and uses disclosed herein comprises (a), at least one nucleic acid sequence comprising at least one gRNA, or any nucleic acid sequence encoding the gRNA; or any kit, composition, vector or vehicle comprising the gRNA or nucleic acid sequence encoding the gRNA. Optionally, the splicing modulating agent may further comprise (b), at least one CRISPR/cas protein, or any nucleic acid molecule encoding the Cas protein, or any kit, composition, vector or vehicle comprising the CRISPR/cas protein or nucleic acid sequence encoding the CRISPR/cas protein, or any nucleic acid sequence encoding said gRNA; or any kit, composition or vehicle comprising at least one of (a) and (b).

Thus, in some embodiments, the Cas protein and the specific gRNA may be provided either as a protein and gRNA, or alternatively, as nucleic acid sequences encoding these two elements, either in two or more separate nucleic acid molecules (e.g., two separate constructs), or comprised in one nucleic acid molecule (e.g., a construct encoding both).

The term “programmable engineered nucleases (PEN)” as used herein also known as “molecular DNA scissors”, refers to enzymes either synthetic or natural, used to replace, eliminate or modify target sequences in a highly targeted way. PEN target and cut specific genomic sequences (recognition sequences) such as DNA sequences. The PEN may be derived from natural occurring nucleases or may be an artificial enzyme, all involved in DNA repair of double strand DNA lesions and enabling direct genome editing. In some alternative or additional embodiments the splicing modulating compound according with the present disclosure encompasses also any nucleic acid molecule comprising at least one nucleic acid sequence encoding the PEN or any kit, composition or vehicle comprising the at least one PEN, or any nucleic acid sequence encoding the PEN. In yet some further specific embodiments, such nucleases may include RNA guided nucleases such as CRISPR-Cas. However, it should be understood that in some alternative embodiments, other nucleases such as ZFN, TALEN, Homing endonuclease, Meganuclease, Mega-TALEN may be used by the methods of the disclosure for targeting at least one target nucleic acid sequence involved in at least one splicing event and inducing aberrant splicing of the target transcript.

More specifically, in some embodiments, the at least one PEN may be at least one of a mega nuclease, a zinc finger nuclease (ZFN), a transcription activator-like effector-based nuclease (TALEN), or a clustered regularly interspaced short palindromic repeats (CRISPR/Cas) system.

In some embodiments, the PEN may be a mega nuclease. Mega nucleases are endodeoxyribonucleases characterized by a large recognition site (double-stranded DNA sequences of 12 to 40 base pairs); such that this site generally occurs only once in any given genome. Meganucleases are specific naturally occurring restriction enzymes and include among others, the LAGLIDADG family of homing endonucleases, mostly found in the mitochondria and chloroplasts of eukaryotic unicellular organisms.

In some embodiments, PEN may be a megaTAL. MegaTALs are fusion proteins that combine homing endonucleases, such as LAGLIDADG family, with the modular DNA binding domains of TALENs.

In some alternative embodiments, the PEN may be a zinc finger nuclease (ZFN). ZFNs are artificial restriction enzymes generated by fusing a zinc finger DNA-binding domain to a DNA-cleavage domain. Zinc finger domains can be engineered to target specific desired DNA sequences, enabling ZFN to target the target sequences within the target transcripts specified by the disclosure, thereby inducing aberrant splicing events.

In yet some other embodiments, the at least one PEN may be a transcription activator-like effector-based nuclease (TALEN). TALEN are restriction enzymes that can be engineered to cut specific sequences of DNA. TALEN are made by fusing a TAL effector DNA-binding domain to a DNA cleavage domain (a nuclease which cuts DNA strands). In yet some further embodiments, additional technologies that can be used are the combination of engineered base editor proteins. These artificially engineered proteins need, just as the CRISPR system discussed above, antisense oligonucleotides to guide the base editor proteins (e.g., any ADAR protein or any variant thereof, or any apolipoprotein B mRNA editing enzyme, catalytic polypeptide-like (APOBEC) protein and any variant thereof), to the proper location that needs to be edited in order to manipulate the splicing reaction.

As indicated above, in some specific embodiments, the targeting of the target nucleic acid sequence that participate in splicing event may be mediated by a PEN that may comprise at least one clustered regulatory interspaced short palindromic repeat (CRISPR)/CRISPR associated (cas) protein system. The Clustered Regularly Interspaced Short Palindromic Repeats (CRISPR) system is a bacterial immune system that has been modified for genome engineering.

CRISPR-Cas systems fall into two major classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI. Thus, in some embodiments, the Cas protein may be a member of at least one of CRISPR-associated system of Class 1 and Class 2. In some embodiments, the cas protein may be a member of at least one of CRISPR-associated system of any one of type II, type I, type III, type IV, type V and type VI from E. coli, Mycobacterium tuberculosis, Haloferax mediterranei, Methanocaldococcus jannaschii, Thermotoga maritima and other bacteria and archaea. It should be understood that the disclosure contemplates the use of any of the known CRISPR systems, particularly any of the CRISPR systems disclosed herein. The CRISPR-Cas system, targets DNA molecules based on short homologous DNA sequences, called spacers that exist between repeats. These spacers guide CRISPR-associated (Cas) proteins to matching sequences within the target DNA, called proto-spacers, which are subsequently cleaved. The spacers can be rationally designed to form guide RNAs (gRNAs) that target any target DNA sequence. It should be noted that the splicing modulating agents of the disclosure comprise in some embodiments at least one gRNA targeted against any of the specific targets specified by the disclosure, or any nucleic acid sequence encoding such gRNA. In some specific embodiment, the RNA guided DNA binding protein nuclease used by the methods of the disclosure may be of a CRISPR Class 2 system. In yet some further particular embodiments, such class 2 system may be a CRISPR type II system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein (RuvC and HNH). However, as the HNH nuclease domain is abundant in restriction enzymes and possesses endonuclease activity responsible for target cleavage.

It should be appreciated that any type II CRISPR-Cas systems may be applicable in the present disclosure, specifically, any one of type II-A, typeII-B or typeII-C. In more particular embodiments, at least one cas protein of type II CRISPR system used by the methods and systems of the disclosure may be the cas9 protein, or any fragments, mutants, fusion proteins, variants or derivatives thereof (e.g., Cas9/Cpf1/CTc(1/2/3), SpCas9, SaCas9, engineered Cas9, and any mutants [for example dCas (with no nuclease activity) e.g., the D10A and/or the H840A that harbor at least one mutation in the RuvC and/or the HNH nucleolytic domains, or any fusion proteins thereof, specifically with any nucleic acid modifying protein, for example, ADAR, as discussed above, with a transcription repressor such as dCAS-KRAB-MeCP2, or alternatively, with a transcription activator, or dCas9-Fok1 (for increasing specificity to the target), and the like]. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occurs, creating the double strand brakes (DSBs).

Still further, CRISPR type II system as used herein requires the inclusion of two essential components: a “guide” RNA (gRNA), that is comprised within the splicing modulating agent of the disclosure, and a non-specific CRISPR-associated endonuclease (Cas9). Guide RNA (gRNA), as used herein refers to a synthetic fusion of the endogenous tracrRNA with a targeting sequence (also named crRNA), providing both scaffolding/binding ability for Cas9 nuclease and targeting specificity. Also referred to as “single guide RNA” or “sgRNA”. In some embodiments, the gRNA of the disclosure may comprise between about 15 to about 50 nucleotides, specifically, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. More specifically, spacers may comprise between about 15 to about 35, 16 to about 35, 17 to about 35, 18 to about 35, 19 to about 35, 20 to about 35, 21 to about 35, 22 to about 35. More specifically, 20-25 nucleotides.

In yet some further embodiments, where the splicing modulating agent comprises at least one nucleic acid sequence encoding the gRNA, such encoding sequence may be designed to target at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more target protospacers (target sequences recognized by the gRNA, for inducing skipping of exon 6 of Fas) within the target transcript or several target transcripts.

In CRISPR systems based on PAM (protospacer adjacent motif) sequence recognition like CRISPR Type II, the PAM is absolutely necessary for target binding and the exact sequence is dependent upon the species of Cas9 used. Thus, in some embodiments, the target sequences within the target transcript, comprise at least one PAM required for recognition and binding of the CRISPR/Cas protein. In certain embodiments, Cas9 from S. pyogenes may be used in the methods, cells, compositions, and kits of the disclosure. In yet some further embodiments, the Cas9 used in the splicing modulating agent may be the S. pyogenes Cas9 protein. In yet some further embodiments the s.p.Cas9 used herein may comprise the amino acid sequence as denoted by SEQ ID NO: 55.

Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. Still further, it should be appreciated that type V CRISPR/Cas, including Cas12a, Cpf1 (type VI), C2C1 (type V-B), Cas13 (type VI), specifically, C2C2 and CasRx and CasX, as well as any variants or fusion proteins thereof, may be also applicable in the disclosure. Still further, in some embodiments, the Cas protein used in the splicing modulating agent of the present disclosure may be any Cas protein known in the art, for example, any one of Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Peil91, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CHI, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

In more specific embodiments, the gRNA comprised within the splicing modulating agent of the disclosure used herein, targets the specific target sequence as disclosed by the disclosure and guides the CRISPR/Cas-protein, specifically, Cas9 to cleave, or perform other modification in the target site. The result of Cas9-mediated DNA cleavage is a double strand break (DSB) within the target DNA. The resulting DSB may be then repaired by one of two general repair pathways, the efficient but error-prone Non-Homologous End Joining (NHEJ) pathway and the less efficient but high-fidelity Homology Directed Repair (HDR) pathway. In some specific embodiments, the targeted nucleic acid sequences specified above are repaired through the NHEJ pathway, resulting in most cases in alteration of the target sequence, also known as indels (insertions/deletions). In some embodiments, the gRNA used in the cells, methods, compositions and uses disclosed herein targets at least one protospacer within at least one of: the 5′ splice site (5′SS) of exon 6 and/or the 3′ splice site (3′SS) of exon 6 of the Fas gene. In some specific embodiments, the gRNA targets at least one protospacer within the 3′SS of exon 6. In some other embodiments, the gRNA targets at least one protospacer within the 5′SS.

In yet some other embodiments, the guide sequence (gRNA) used in the cells, methods, compositions and uses disclosed herein, may comprise the nucleic acid sequence as denoted by SEQ ID NO: 15 or any variants and/or derivatives thereof. The full sgRNA sequence used in the cells, methods, compositions and uses disclosed herein, may comprise the nucleic acid sequence as denoted by SEQ ID NO: 16 or any variants and/or derivatives thereof. The 3′ splice site targeted sequence in FAS is as denoted by SEQ ID NO: 17 (comprising the intron5 exon6 junction sequence) or as denoted by SEQ ID NO: 18 (the complementary sequence to the gRNA as denoted by SEQ ID NO: 15). The enhanced skipping of exon 6 of the Fas gene results in a product that lacks exon 6 of Fas. In some specific embodiments, such product may be the soluble Fas, also referred to herein as the sFas, or as the FASΔEX6. In some specific and non-limiting embodiments, the resulting product that lacks exon 6 may comprise the amino acid sequence as denoted by SEQ ID NO: 35, or any variants and mutants thereof. As indicated herein, when using a gene editing system as a splicing modulating agent, due to indels or any other point mutations or variations that may be introduced during the aberrant splicing event, the resulting sFAS, or FASΔEX6, may comprise a variant, mutant or derivative of the amino acid sequence as denoted by SEQ ID NO: 35.

Thus, in some embodiments, the non-naïve cell of the T lineage according to the present disclosure express reduced levels of membrane Fas (mFas). In some specific embodiments, the mFas may comprise the amino acid sequence as denoted by SEQ ID NO: 34.

Thus, in some embodiments, an enhanced skipping of exon 6 of the Fas gene may result in an increase, elevation, enhancement, of about 5%, 10%, 15%, 20%, 25%, 30%, 35%, 40%, 45%, 50%, 55%, 60%, 65%, 70%, 75%, 80%, 85%, 90%, 95%, 99%, or more specifically 100% of the resulting sFAS, or FASΔEX6, as compared to exon skipping that occurs naturally with no manipulation as discussed herein. Similarly, the enhanced skipping of exon 6 of the Fas gene may result in a decrease, reduction of about 5%, 10%, 15%, 20%, 25%, 30%, 35%, 40%, 45%, 50%, 55%, 60%, 65%, 70%, 75%, 80%, 85%, 90%, 95%, 99%, or more specifically 100% of the mFAS produced, as compared to exon skipping that occurs naturally with no manipulation as discussed herein.

In some other embodiments, the non-naïve cell of the T lineage according to the present disclosure may be further engineered to express at least one receptor molecule. In some embodiments such receptor may comprise at least one target binding domain specific against at least one target antigen.

As indicated above, it should be appreciated that the splicing modulating agent comprising at least one nucleic acid sequence used by the methods of the invention can be expressed from a nucleic acid construct administered to the individual or contacted with the target cells employing any suitable mode of administration (i.e., in-vivo gene therapy). Alternatively, the nucleic acid construct is introduced into a suitable cell via an appropriate gene delivery vehicle/method (transfection, transduction, homologous recombination, etc.).

As used herein, a cell has been “transformed”, “transduced” or “transfected” by exogenous or heterologous DNA and/or RNA, e.g., the splicing modulating agent that may comprise nucleic acid molecule/s or any cassette or system comprising the same, when such DNA/RNA has been introduced inside the cell. The transforming DNA may be integrated (covalently linked) into the genome of the cell. With respect to the present invention, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. It should be appreciated that the present disclosure further encompasses any population of cells comprising at least 5%, 10%, 15%, 20%, 25%, 30%, 35%, 40%, 45%, 50%, 55%, 60%, 65%, 70%, 75%, 80%, 85%, 90%, 95%, 99.9% or more, specifically, 100% of the manipulated cells of the preset disclosure.

In some embodiments, the receptor molecule may be at least one of: (a) at least one T-cell receptor (TCR) molecule specific for at least one target antigen; and/or (b) at least one chimeric antigen receptor (CAR) molecule specific for at least one target antigen.

In some embodiments, the cells of the present disclosure may further express at least one receptor molecule. The receptor molecule may be either endogenous molecule or alternatively, exogeneous molecule exogenously added to the cell, for example, by genetically editing the cell. In some embodiments, the receptor molecule may be at least one CAR molecule. The term “chimeric protein” relates to proteins created through the joining/fusing of two or more genes that originally coded for separate proteins. Translation of this chimeric/fusion gene results in a single or multiple polypeptides with functional properties derived from each of the original proteins. Recombinant chimeric/fusion proteins are created artificially by recombinant DNA technology. Chimeric or chimera usually designate hybrid proteins made of polypeptides having different functions, sources or physico-chemical patterns.

Chimeric Antigen Receptor (CAR), as used herein, refers to a polypeptide comprising at least an extracellular antigen binding domain, a transmembrane domain and an intracellular cytoplasmic signaling domain comprising a functional stimulatory domain.

The receptors are chimeric because they couple between extracellular antigen-binding capabilities and intracellular T-cell, activating functions, in a single receptor molecule. CARs have been engineered to give the cells they are expressed in a new ability to recognize a specific antigen of interest, thereby facilitating an immune reaction against it. For example, the technology is used in immunotherapy for specifically recognizing specific cancer cells' antigens of interest in order to more effectively direct the immune cells towards those target cells and destroy them.

As indicated above, as being engineered receptors, CARs graft an arbitrary specificity onto an immune effector cell. Typically, these receptors are used to graft the specificity of a monoclonal antibody onto a T cell.

The initial design (also referred to a first generation) joined an antibody-derived scFv to the CD3ζ intracellular signaling domain of the T-cell receptor through hinge and transmembrane domains.

Second generation CARs add intracellular signaling domains from various costimulatory protein receptors (e.g., CD28, 41BB, ICOS) to the cytoplasmic tail of the CAR to provide additional signals to the T cell. More recent, third generation CARs combine multiple signaling domains, such as CD27, CD28, 4-1BB, ICOS, or OX40, to augment potency.

More specifically, in some embodiments, the non-naïve cell of the T lineage of the present disclosure further expresses at least one receptor molecule, specifically, at least one CAR molecule. In some embodiments such CAR molecule may comprise: (i) at least one target-binding domain that specifically recognizes and binds at least one target antigen; (ii) at least one hinge and at least one transmembrane domain; and (iii) at least one intracellular T cell signal transduction domain.

In some embodiments, the at least one target binding domain of the CAR molecule that is further expressed by the non-naïve cell of the T lineage of the present disclosure, may comprise at least one antibody or any antigen-binding fragment/s, portion/s or chimera/s thereof, specific for a target antigen.

The CAR molecule provided herein, comprises at least one target-binding domain, that may be in some embodiments, any target-recognition element, for example, at least one antibody or any antigen-binding fragments or domains thereof. In yet some further embodiments, the target-recognition element of the CAR molecule of the present disclosure comprises at least one antibody or any antigen-binding fragment/s, portion/s or chimera/s thereof.

It should be understood that the non-naïve cell of the T lineage in accordance with the present disclosure may further express any CAR molecule directed against any target molecule. In some specific and non-limiting embodiments, the non-naïve cell of the T lineage in accordance with the present disclosure may further express a CAR molecule directed against the CD19 molecule. The CD19 molecule is present in B-cell-derived cancers such as acute lymphoblastic leukemia (ALL) and diffuse large B-cell lymphoma (DLBCL).

Still further, in some embodiments, the non-naïve cell of the T lineage of in accordance with the present disclosure may further express at least one CAR molecule directed against the CD30 molecule.

In some embodiments, the non-naïve cell of the T lineage in accordance with the present disclosure may further express at least one at least one CAR molecule directed against the CD33 molecule.

In some embodiments, the non-naïve cell of the T lineage in accordance with the present disclosure may further express at least one CAR molecule directed against the CD123 molecule.

In some embodiments, the non-naïve cell of the T lineage in accordance with the present disclosure may further express at least one CAR molecule directed against the FLT3 molecule.

In some embodiments, the non-naïve cell of the T lineage in accordance with the present disclosure may further express at non-naïve cell of the T lineage at least one CAR molecule directed against the BCMA molecule.

In yet some further additional or alternative embodiments, the non-naïve cell of the T lineage in accordance with the present disclosure may further express at least one T cell receptor targeted against at least one target antigen. More specifically, the T-cell receptor (TCR) is a protein complex found on the surface of T cells, or T lymphocytes, that is responsible for recognizing fragments of antigen as peptides bound to major histocompatibility complex (MHC) molecules. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. The core TCR complex consists of two TCR chains and six cluster of differentiation 3 (CD3) chains. The human genome expresses four TCR genes known as TCRα, TCRβ, TCRγ, and TCRδ, which forms two distinct heterodimers: TCRα/TCRβ or TCRγ/TCRδ. The majority of mature T cells expresses TCRα and TCRβ isoforms, generally referred to as T cells (or αβ T cells), while a small portion (0.5-5%) of T lymphocytes (γδ T cells) expresses TCRγ and TCRδ isoforms. Both heterodimers form multiprotein complexes with CD3 δ, γ, ε, and ζ chains. However, in both complexes, three dimers of CD3 proteins, δε and γε heterodimers and ((homodimers, are present. These CD3 proteins associate with TCR via non-covalent hydrophobic interactions and are required for a complete TCR localization on the cell surface. The TCR mediates recognition of antigenic peptides bound to MHC molecules (pMHC), whereas the CD3 molecules transduce activation signals to the T cell.

In some embodiments, the at least one target antigen targeted by the TCR or the CAR molecules further expressed by the non-naïve cell of the T lineage of the present disclosure, may be at least one of: at least one tumor associated antigen (TAA), at least one tumor specific antigen (TSA), at least one neoantigen, at least one viral antigen, at least one bacterial antigen, at least one fungal antigen and/or at least one parasite antigen.

It should be understood that the at least one receptor molecule comprising the at least one target binding domain that may be directed to any antigen of interest, specifically any antigen specific for a pathologic disorder. In more specific embodiments, the receptor molecule expressed either endogenously or exogenously by the non-naïve cell of the T lineage, may be directed against antigens specific for proliferative disorders, specifically, tumor associated antigens (TAAs), tumor specific antigen (TSA), or antigens specific for any pathogen, specifically, viral, bacterial, fungal or parasitic pathogen. Specific pathogens applicable in the present disclosure, are described in more detail herein after. In some specific embodiments, the at least one receptor molecule disclosed in the disclosure, may comprise at least one antibody directed against at least one tumor associated antigen (TAA).

Tumor or cancer associated antigen (TAA), as used herein may be an antigen that is specifically expressed, over expressed or differentially expressed in tumor cells. In yet some further embodiments, TAA can stimulate tumor-specific T-cell immune responses. Exemplary tumor antigens that may be applicable in the present disclosure, include, but are not limited to, Melan-A/MART-1, RAGE-1, tyrosinase, MAGE-1, MAGE-2, NY-ESO-1, glycoprotein (gp) 75, gp100, MUC1, beta-catenin, PRAME, MUM-1, WT-1, CEA, PR-1 CD45, glypican-3, IGF2B3, Kallikrein4, KIF20A, Lengsin, Meloe, MUC5AC, survivin, CLPP, Cyclin-A1, SSX2, XAGE1b/GAGED2a, MAGE-A3, MAGE-A6, LAGE-1, CAMEL, hTRT and Eph, and TRP-1. Still further, TAA may be recognized by CD8+ T cells as well as CD4+ T cells. Non limiting examples of TAA recognized by CD8+ T cells may be CSNK1A1, GAS7, HAUS3, PLEKHM2, PPPIR3B, MATN2, CDK2, SRPX (P55L), WDR46 (T2271), AHNAK (S4460F), COL18A1 (S126F), ERBB2 (H197Y), TEAD1 (L209F), NSDHL (A290V), GANAB (S184F), TRIP12 (F1544S), TKT (R438W), CDKN2A (E153K), TMEM48 (F169L), AKAP13 (Q285K), SEC24A (P469L), OR8B3 (T190I), EXOC8 (Q656P), MRPS5 (P59L), PABPC1 (R520Q), MLL2, ASTN1, CDK4, GNL3L, SMARCD3, MAGE-A6, MED13, PAS5A WDR46, HELZ2, AFMID, CENPL, PRDX3, FLNA, KIF16B, SON, MTFR2 (D626Y), CHTF18 (L769V), MYADM (R30W), NUP98 (A359D), KRAS (G12D), CASP8 (F67V), TUBGCP2 (P293L), RNF213 (N1702S), SKIV2L (R653H), H3F3B (A48T), AP15 (R243Q), RNF10 (E572K), PHLPP1 (G566E) and ZFYVE27 (R6H). Non limiting examples of TAA recognized by CD4+ T cells may be ERBB2IP (E805G), CIRH1A (P333L), GART (V551A), ASAP1 (P941L), RND3 (P49S), LEMD2 (P495L), TNIK (S502F), RPS12 (V104I), ZC3H18 (G269R), GPD2 (E426K), PLEC (E1179K), XPO7 (P274S), AKAP2 (Q418K) and ITGB4 (S10021). Non-limiting examples of MHC class II-restricted antigens may be Tyrosinase, gp100, MART-1, MAGE-A1, MAGE-A2, MAGE-A3, MAGE-A6, LAGE-1, CAMEL, NY-ESO-1, hTRT and Eph. In some embodiments, the TAA may be CD30 (in refractory Hodgkin's lymphoma). In yet some further embodiments, the TAA may be CD33. In some embodiments, the TAA may be CD123. In some embodiments, the TAA may be FLT3 that is expressed in in acute myeloid leukemia (AML). In some embodiments, the TAA may be BCMA that is expressed in multiple myeloma. In some embodiments, the TAA may be MESOTHELIN that is expressed in lung, pancreatic cancer. In some embodiments, the TAA may be GD2 that is expressed in melanoma, brain, pancreatic cancers. In some embodiments, the TAA may be CEA that is expressed in Colorectal cancer. In some embodiments, the TAA may be HER2 that is expressed in in breast cancer. In some embodiments, the TAA may be CEACAM 7 that is expressed in Pancreatic cancer. In some embodiments, the TAA may be CD22 expressed in Lymphoma. In some embodiments, the TAA may be mutated p53. Still further in some embodiments, the TAA may be gp100 that is expressed in Melanoma. In some embodiments, the TAA may be Tyrosinase (Melanoma). In some embodiments, the TAA may be NY-ESO1 (Epithelial malginancies). In some embodiments, the TAA may be MAGE antigen family. In some embodiments, the TAA may be WT1 (hematological malignancies). In some embodiments, the TAA may be MUC-1 (Breast ovarian carcinoma, pancreatic cancer).

In some embodiments, the TAA may be LMP2 (EBV-linked malignancies). In some embodiments, the TAA may be E6, E7 (HPV-linked malignancies).

Cancer antigen and tumor antigen are used interchangeably herein. The antigens may be related to cancers that include, but are not limited to, skin cancer, hematological malignancies, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (e.g., nonmelanoma, melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

Still further, in some embodiments, a few examples of antibodies used in the treatment of cancer that may be applicable in the present disclosure (e.g., the target recognition moiety of the CAR or the engineered TCR expressed by the non-naïve cells of the T lineage of the present disclosure. include, but are not limited to monoclonal antibodies such as Bevacizumab (UNII: 2S9ZZM9Q9V), Cetuximab (UNII: PQX0D8J21J), Panitumumab (UNII: 6A901E312A), Rituximab (UNII: 4F4X42SYQ6), Alemtuzumab (UNII 3A189DH42V), Trastuzumab (UNII: P188ANX8CKO, that is directed against HER2, Ipilimumab (UNII: 6T8C155666, Yervoy), that is a check point inhibitor, specifically, a monoclonal antibody that works to activate the immune system by targeting CTLA-4, Tremelimumab, formerly ticilimumab (CP-675,206) is a fully human monoclonal antibody against CTLA-4, ibritumomab tiuxetan (UNII: 4Q52C550XK), lambrolizumab (formerly MK-3475, Pembrolizumab, Keytruda® UNII: DPT003T46P), that is a check point inhibitor, specifically, a humanized antibody that targets programmed cell death (PD-1), Nivolumab (Opdivo® UNII: 31YO63LBSN) is an Fab fragment of an antibody that binds the extracellular domain of PD-1, Atezolizumab (trade name Tecentriq) is a fully humanized, engineered monoclonal antibody of IgG1 isotype against the protein programmed cell death-ligand 1 (PD-L1), Avelumab (trade name Bavencio) is a fully human monoclonal antibody that targets PD-L1, Durvalumab is a human immunoglobulin G1 kappa (IgG1κ) monoclonal antibody that blocks the interaction of PD-L1 with the PD-1 and CD80 (B7.1) molecules and Tremelimumab (formerly ticilimumab; UNII: QEN1X95CIX) that is a check point inhibitor and ado-trastuzumab emtansine (UNII: SE2KH7T06F).

In some embodiments, the at least one target antigen recognized by the receptor further expressed by the non-naïve cell of the T lineage, may be a tumor specific antigen.

A tumor-specific antigen (TSA) is a protein or other molecule that is produced by cancer cells and not normally found in healthy cells. There are different types of TSAs, including those that are specific to a particular type of cancer (such as prostate-specific antigen for prostate cancer) and those that are more broadly expressed across different types of cancer (such as carcinoembryonic antigen). Major classes of TSAs, include those generated from mutational frameshifts, splice variants, gene fusions, endogenous retroelements and other classes, such as human leukocyte antigen (HLA)-somatic mutation-derived antigens and post-translational TSAs. Non-limiting examples for TSAs useful in the present disclosure may include, but are not limited to mutated p53, mutated Ras, various splice variants (e.g., RHAMM-48 and RHAMM-147), BCR-ABL fusion and the like. In some particular embodiments, receptors optionally expressed in the non-naïve cell of the T lineage of the present disclosure may be directed against any antigen derived from a pathogen, specifically, viral, bacterial, fungal, parasitic pathogen and the like. Thus, in some specific embodiments, the at least one receptor expressed by the non-naïve cell of the T lineage, may be directed against a viral antigen. It should be appreciated that any of the viral pathogens discussed herein after, is applicable in this aspect, as well as in all aspects of the present disclosure.

In some specific embodiments, the viral pathogen may be of any of the following orders, specifically, Herpesvirales (large eukaryotic dsDNA viruses), Ligamenvirales (linear, dsDNA (group I) archaean viruses), Mononegavirales (include nonsegmented (−) strand ssRNA (Group V) plant and animal viruses), Nidovirales (composed of (+) strand ssRNA (Group IV) viruses), Ortervirales (single-stranded RNA and DNA viruses that replicate through a DNA intermediate (Groups VI and VII)), Picornavirales (small (+) strand ssRNA viruses that infect a variety of plant, insect and animal hosts), Tymovirales (monopartite (+) ssRNA viruses), Bunyavirales contain tripartite (−) ssRNA viruses (Group V) and Caudovirales (tailed dsDNA (group I) bacteriophages).

In yet some further specific embodiments, the receptors optionally expressed in the non-naïve cell of the T lineage of the present disclosure may be specifically directed against DNA viruses, specifically, any virus of the following families: the Adenoviridae family, the Papovaviridae family, the Parvoviridae family, the Herpesviridae family, the Poxviridae family, the Hepadnaviridae family and the Anelloviridae family.

In yet some further specific embodiments, the receptors optionally expressed in the non-naïve cell of the T lineage may be specifically directed against RNA viruses, specifically, any virus of the following families: the Reoviridae family, Picomaviridae family, Caliciviridae family, Togaviridae family, Arenaviridae family, Flaviviridae family, Orthomyxoviridae family, Paramyxoviridae family, Bunyaviridae family, Rhabdoviridae family, Filoviridae family, Coronaviridae family, Astroviridae family, Bomaviridae family, Arteriviridae family, Hepeviridae family and the Retroviridae family.

In more specific embodiments, the receptors optionally expressed in the non-naïve cell of the T lineage of the present disclosure may be directed against any antigen derived from a viral pathogen of the order Mononegavirales. In yet some further embodiments, receptors optionally expressed in the non-naïve cell of the T lineage may be directed against an antigen derived from a virus of the family Pneumoviridae. In more specific embodiments, the receptors optionally expressed in the non-naïve cell of the T lineage may be directed against any antigen derived from a viral pathogen of the genus Orthopneumovirus. In some specific embodiments, such viral antigen may be an antigen specific for respiratory syncytial virus (RSV), for example, any one of the Human respiratory syncytial virus (HRSV), A2 and B1, the bovine respiratory syncytial virus (BRSV) and the murine pneumonia virus (MPV). In more specific embodiments, the receptors optionally expressed in the non-naïve cell of the T lineage may be directed against the human RSV. In yet some further specific embodiments, the anti-RSV antibody may be the anti-RSV palivizumab antibody. More specifically, Palivizumab (brand name Synagis, manufactured by MedImmune) is a humanized monoclonal antibody (IgG) directed against an epitope in the A antigenic site of the F protein of RSV.

In yet some further specific embodiments, the receptors optionally expressed by the non-naïve cell of the T lineage may be directed against any antigen derived from a viral pathogen of the family Retroviridae. In yet some further embodiments, the receptors optionally expressed in the non-naïve cell of the T lineage may be directed against an antigen derived from a virus of the subfamily Orthoretrovirinae. In more specific embodiments receptors optionally expressed in the non-naïve cell of the T lineage may be directed against any antigen derived from a viral pathogen of the genus Lentivirus, specifically, of the species human immunodeficiency virus (HIV).

Specific embodiments that relate to particular viruses associated with specific disorders are specified herein below. It should be understood that any of the viral pathogens and any of the bacterial, fungal and parasite pathogen described herein after, are also applicable in connection with the antigens derived therefrom that are recognized by the receptors optionally expressed in the non-naïve cell of the T lineage of the present disclosure.

In more specific embodiments, the receptors optionally expressed in the non-naïve cell of the T lineage may be directed against at least one neoantigen. A neoantigen, as used herein, is a type of antigen created by a genetic mutation, alternative splicing or other alteration in a cell, for example, a cancer cell. Neoantigens are not present in normal, healthy cells, making them an attractive target for cancer immunotherapy. Neoantigens are formed when a mutation occurs in regulatory or encoding sequences leading to the creation of a novel protein or a modification of an existing protein. The mutated protein is then presented on the surface of the cell as an antigen, which can be recognized by the immune system as foreign.

In some specific embodiments, the antigen recognized by the TCR or CAR molecule that is further expressed by the non-naïve cell of the T lineage of the present disclosure, is a melanoma antigen. In yet some further embodiments, the melanoma antigen may be the MART-1 antigen as disclosed herein above.

In some embodiments, the non-naïve cell of the T lineage manipulated for enhanced skipping of exon 6 of the Fas according to the present disclosure is characterized by at least one of (i) reduced expression of mFas; (ii), increased cytokine secretion; (iii) increased expression of activation markers; (iv) increased cell survival; (v) increased cytotoxicity; and/or (vi) reduced expression of exhaustion markers.

In yet some further embodiments the non-naïve cell of the T lineage manipulated for enhanced skipping of exon 6 of the Fas according to the present disclosure may be characterized by increased mitochondrial production and/or increased resistance to FASL, increased or decreased CD4:CD8 ratios and increase tumor immunity.

The disclosed non-naïve cell of the T lineage manipulated according to the present disclosure display a reduced expression of membrane Fas (mFas) as compared with control cells, specifically cells that are not manipulated for enhanced skipping of exon 6 of the Fas. The reduced expression of mFas is caused directly or indirectly by the initial manipulation of the disclosed cells for enhanced skipping of exon 6 in Fas. The reduced expression of mFas may be in the mRNA and/or protein levels.

According to some embodiments, the disclosed non-naïve cell of the T lineage manipulated according to the present disclosure display an increased activation as compared with control cells, specifically cells that are not manipulated to enhanced skipping of exon 6 of the Fas. As indicated above, increased activation of T cells may be reflected for example, by the increase in the expression of activation markers. Activation markers refer herein to molecules which are upregulated upon T cell activation, each at a different stage of the activation process.

According to some embodiments, increase in the expression of activation markers, may comprise for example, increase in the expression of at least one of CD25, 4-1BB and/or CD40L, as compared with control cells, specifically cells that are not manipulated to enhanced skipping of exon 6 of the Fas.

In yet some further embodiments, the non-naïve cell of the T lineage manipulated according to the present disclosure display reduced expression of at least one exhaustion markers as compared with control cells, specifically cells that are not manipulated to enhanced skipping of exon 6 of the Fas. This reduced exhaustion is reflected by reduced expression of exhaustion markers in response to a specific stimulation. T cell Exhaustion as used herein refers to a state of T cell dysfunction that arises during many chronic infections and cancer. It is defined by poor effector function, sustained expression of inhibitory receptors and a transcriptional state distinct from that of functional effector or memory T cells. By “dysfunction” here it is understood that some T cells, after activation and proliferation, do not fulfill the functions they are expected to perform as effector T cells—typically, they fail to eliminate cancerous or infected cells and control the tumor or the virus respectfully. As originally described, antigen-specific T cells become “dysfunctional” during the chronic phase of high viral load infections, with progressive loss of interleukin (IL)-2, then tumor necrosis factor alpha (TNFα), and, finally, interferon gamma (IFNγ). Thus, in some embodiments, the disclosed non-naïve cell of the T lineage manipulated according to the present disclosure display or characterized by reduced expression of exhaustion markers. More specifically, in some embodiments, the exhaustion markers may be at least one of Programmed Death-1 receptor (PD-1), Lymphocyte activation gene-3 (LAG-3, is also named CD223 or FDC protein), T-cell immunoglobulin and mucin-domain containing-3 (TIM3), T cell immunoreceptor with Ig and ITIM domains (TIGIT). Still further, in some additional and non-limiting embodiments, exhaustion markers applicable in the present disclosure include inducible T-cell co-stimulator (ICOS), cytotoxic T-lymphocyte-associated protein-4 (CTLA-4), CD244 (2B4), CD160, killer cell lectin-like receptor subfamily G member 1 (KLRG1), and the like.

In yet some further embodiments, the non-naïve cell of the T lineage manipulated according to the present disclosure display increased cytokine secretion as compared with control cells, specifically cells that are not manipulated to enhanced skipping of exon 6 of the Fas. Cytokine secretion refers to the process by which cells release cytokines into the surrounding tissue or media. Cytokines, in accordance with the present disclosure include interleukins, for example, interferon gamma (IFNγ), soluble FAS, granzyme B (GrB), interleukine-2, (IL-2), interleukin-1 (IL-1) and interleukin-6 (IL-6), Tumor necrosis factor (TNF), Chemokines, such as Transforming growth factor-beta (TGF-beta) and the like.

Still further, in some embodiments, the non-naïve cell of the T lineage manipulated according to the present disclosure display increased cell survival as compared with control cells, specifically cells that are not manipulated to enhanced skipping of exon 6 of the Fas.

Increased cell survival refers to the ability of cells to withstand stress or damage and continue to survive and function normally. This can be achieved through various mechanisms, such as increased resistance to oxidative stress, DNA damage, or other forms of cellular stress, as well as enhanced repair mechanisms that allow damaged cells to recover and continue to function. It should be appreciated that increased cell survival and/or viability may be monitored by any routine procedure enabling live cell counting, e.g., MTT, MTS, XTT, WSF1 tests or any assay based on protease activity (relative viability assays).

In yet some further embodiments, the non-naïve cell of the T lineage manipulated according to the present disclosure display increased T cell cytotoxicity as compared with control cells, specifically cells that are not manipulated to enhanced skipping of exon 6 of the Fas. Increased T cell cytotoxicity refers to the ability of T cells to efficiently target and eliminate specific target cells. Cytotoxic T cells (also known as CD8+ T cells) recognize and bind to specific antigens presented on the surface of target cells and then release cytotoxic molecules, such as perform and granzyme, to induce cell death. Increased T cell cytotoxicity can be achieved through various mechanisms, such as upregulation of cytotoxic molecules, increased expression of activation markers, or enhanced differentiation and proliferation of T cells. Assays for evaluation of increased cytotoxicity of the cells may include any functional assay such as lactate dehydrogenase assay, homogeneous membrane integrity assay, DNA dye cytotoxicity assays and the like.

In yet some further embodiments, the non-naïve cell of the T lineage manipulated according to the present disclosure display increased cell proliferation as compared with control cells, specifically cells that are not manipulated to enhanced skipping of exon 6 of the Fas. Increased cell proliferation refers to an increase in the rate at which cells divide and multiply to produce new cells. Cell proliferation is regulated by a complex network of signaling pathways and checkpoints that ensure proper cell cycle progression and prevent uncontrolled growth.

The disclosed non-naïve cell of the T lineage manipulated for enhanced skipping of exon 6 of the Fas display in some embodiments increased and elevated activity that is indicated in several functional and structural parameters. Increase, as used herein, in connection with various improved properties of the manipulated T-cell of the present disclosure, is meant that such increase or enhancement may be an increase or elevation or improvement of the indicated feature/activity (e.g., cytokine secretion, expression of activation markers, survival, proliferation, and the like), of between about 1% to 100%, specifically, 5% to 100% of the indicated parameter, more specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more, as compared with control cells, specifically cells that are not manipulated for enhanced skipping of exon 6 of the Fas. In yet some further embodiments, the terms “inhibition”, “moderation”, “reduction”, “decrease” or “attenuation” as referred to herein with respect to the various properties of the non-naïve cell of the T lineage manipulated according to the present disclosure (e.g., expression of the mFas, expression of exhaustion markers), relate to the retardation, restraining or reduction of the indicated parameter by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more, as compared with control cells, specifically cells that are not manipulated for enhanced skipping of exon 6 of the Fas. More specifically, the terms “enhance”, “increase”, “augmentation” and “enhancement” as used herein relate to the act of becoming progressively greater in size, amount, number, or intensity. Alternatively, “inhibition”, “moderation”, “reduction”, “decrease” or “attenuation” as used herein relate to the act of becoming progressively smaller in size, amount, number, or intensity. Particularly, an increase or alternatively, decrease of 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the indicated activity (e.g., increase of cytokine secretion and/or expression of activation markers, or alternatively, decrease of expression of exhaustion markers), as compared to a suitable control, e.g., cells that are not manipulated for enhanced skipping of exon 6 of the Fas.

In some embodiments, the indicated results of the enhanced skipping of exon 6 of the Fas in the disclosed manipulated cells may be in response to a specific stimulation in an in vivo and/or in vitro ex vivo setting, for example, activation of T-cells (e.g., by anti-CD3 antibodies and the like.

Still further, in some particular and non-limiting embodiments, the disclosed cells may be further manipulated prior, during or after manipulating of the exon skipping using the splicing modulating agents of the present disclosure.

In some specific embodiments, the cells are activated prior to manipulation of the exon skipping, e.g., prior to contacting the cells with the splicing modulating agents (e.g., SSOs, gRNAs) as discussed herein. Activation may include but is not limited to contacting the cells with an anti-CD3 antibody and/or with anti-CD28 antibody, and/or with any specific and non-specific activation component. In yet some further embodiments, the activation may encompass contacting the cells with Phorbol myristate acetate (PMA) and ionomycin. In some alternative or additional embodiments, activation may be performed using antigen peptide.

A further aspect of the preset disclosure relates to a composition comprising at least one of. (a), at least one non-naïve cell of the T lineage manipulated for enhanced skipping of exon 6 of the Fas gene, or a cell population comprising at least one of the cell. The non-naïve manipulated cell of the T lineage according to the present disclosure predominantly expresses the soluble product of Fas (sFas, FASΔEX6). In yet some alternative or additional embodiments, the disclosed composition may further comprise and/or alternatively comprise (b), at least one splicing modulating agent or any vector, vehicle, matrix, nano- or micro-particle or composition comprising the at least one agent. The splicing modulating agent comprises at least one nucleic acid molecule that targets at least one target nucleic acid sequence participating directly or indirectly in at least one splicing event of the Fas gene. In some embodiments, the composition optionally further comprising at least one of pharmaceutically acceptable carrier/s, diluent/s, excipient/s and additive/s.

In some embodiments, the non-naïve cell of the T lineage in the composition is as defined by the present disclosure.

The pharmaceutical compositions of the disclosure can be administered and dosed by the methods of the disclosure, in accordance with good medical practice, systemically, for example by parenteral, e.g., intrathymic, into the bone marrow and intravenous. It should be noted however that the disclosure may further encompass additional administration modes, especially in embodiments where the composition comprising the splicing modulating agent/s). In other examples, the pharmaceutical composition can be introduced to a site by any suitable route including intraperitoneal, subcutaneous, transcutaneous, topical, intramuscular, intraarticular, subconjunctival, or mucosal, e.g., oral, intranasal, or intraocular administration.

Local administration to the area in need of treatment may be achieved by, for example, by local infusion during surgery, topical application, direct injection into the specific organ (bone marrow, spleen, lymph nodes), etc. More specifically, the compositions used in any of the methods of the disclosure, described herein, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

More specifically, pharmaceutical compositions used to treat subjects in need thereof according to the disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, nucleic acid molecule/s of the disclosure or any cassette/s thereof, with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present disclosure also include, but are not limited to, emulsions and liposome-containing formulations. It should be understood that in addition to the ingredients particularly mentioned above, the formulations may also include other agents conventional in the art having regard to the type of formulation in question. Still further, pharmaceutical preparations are compositions that include one or more nucleic acid molecules, vectors and/or cassette and/or cells of the present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle”, when referred to the compositions in the present aspect, refers to a diluent, adjuvant, excipient, or carrier with which a compound of the disclosure is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the nucleic acid molecule/s that targets at least one target nucleic acid sequence participating directly or indirectly in at least one splicing event of the Fas gene and optionally, the at least one receptor molecule of the disclosure or any non-naïve cell of the T lineage manipulated for enhanced skipping of exon 6 of the Fas, and systems of the disclosure, can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity, or it may be formulated for sustained release.

In numerous embodiments, the compositions of the present disclosure may be administered in a form of combination therapy, i.e. in combination with one or more additional therapeutic agents. Combination therapy may include administration of a single pharmaceutical dosage formulation comprising at least one composition of the disclosure and additional therapeutics agent(s); as well as administration of at least one composition of the disclosure and one or more additional agent(s) in its own separate pharmaceutical dosage formulation. Further, where separate dosage formulations are used, compositions of the disclosure and one or more additional agents can be administered concurrently or at separately staggered times, i.e. sequentially. Still further, the concurrent or separate administrations may be carried out by the same or different administration routes. Thus, in some further embodiments, the non-naïve cell of the T lineage manipulated for enhanced skipping of exon 6 of the Fas of the present disclosure may be applicable in boosting the immune response of a subject suffering from a pathologic disorder and may be used in combined treatment with any therapeutic agent, for example, a chemotherapeutic agent.

As used herein, a “chemotherapeutic agent” or “chemotherapeutic drug” (also termed chemotherapy) as used herein refers to a drug treatment intended for eliminating or destructing (killing) cancer cells or cells of any other proliferative disorder. The mechanism underlying the activity of some chemotherapeutic drugs is based on destructing rapidly dividing cells, as many cancer cells grow and multiply more rapidly than normal cells. As a result of their mode of activity, chemotherapeutic agents also harm cells that rapidly divide under normal circumstances, for example bone marrow cells, digestive tract cells, and hair follicles. Insulting or damaging normal cells result in the common side-effects of chemotherapy: myelosuppression (decreased production of blood cells, hence also immuno-suppression), mucositis (inflammation of the lining of the digestive tract), and alopecia (hair loss).

Various different types of chemotherapeutic drugs are available. A chemotherapeutic drug may be used alone or in combination with another chemotherapeutic drug or with other forms of cancer therapy, in addition to the non-naïve cell of the T lineage manipulated according to the present disclosure, for example, other biological drugs (antibodies, ligands, receptors), radiation therapy or surgery.

Chemotherapeutic drugs affect cell division or DNA synthesis and function and can be generally classified into several groups, based on their structure or biological function. More specifically, chemotherapeutic agents that are classified as alkylating agents, anti-metabolites, anthracyclines, plant alkaloids, topoisomerase inhibitors, and other anti-tumor agents such as DNA-alkylating agents, anti-tumor antibiotic agents, tubulin stabilizing agents, tubulin destabilizing agents, hormone antagonist agents, protein kinase inhibitors, HMG-CoA inhibitors, checkpoint inhibitors, CDK inhibitors, cyclin inhibitors, caspase inhibitors, metalloproteinase inhibitors, antisense nucleic acids, triple-helix DNAs, nucleic acids aptamers, and molecularly-modified viral, bacterial or exotoxic agents. It should be appreciated that any combination therapy disclosed herein, using any of the indicated compounds with the cells and compositions of the present disclosure, together with any of the therapeutic agents discussed above, is encompassed by the present disclosure.

Still further, compositions and formulations for oral administration may include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable. Pharmaceutical formulations adapted for rectal administration may be presented as suppositories or enemas. Pharmaceutical formulations adapted for vaginal administration may be presented as pessaries, tampons, creams, gels, pastes, foams or spray formulations.

Pharmaceutical compositions used to treat subjects in need thereof according to the disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present disclosure also include, but are not limited to, emulsions and liposome-containing formulations. It should be understood that in addition to the ingredients particularly mentioned above, the formulations may also include other agents conventional in the art having regard to the type of formulation in question, for example those suitable for oral administration may include flavoring agents. The compositions of the disclosure may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose or methyl cellulose or a heteropolysaccharide polymer, for example, gelan gum, may be incorporated together with a preservative, such as benzalkonium chloride. Such formulations may also be delivered by iontophoresis. Formulations for ocular and aural administration may be formulated to be immediate and/or modified release. Modified release includes delayed, sustained, pulsed, controlled, targeted, and programmed release. In specific embodiments, the unit dosage formulations are those containing a daily dose or sub-dose, as herein above recited, or an appropriate fraction thereof, of an active ingredient. It should be appreciated that the formulations of the compositions of the disclosure may be in some embodiments, adapted for use as a nano- or micro-particles. Nanoscale drug delivery systems using liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds. Externally controlled release can be accomplished if drug delivery vehicles, such as liposomes or polyelectrolyte multilayer capsules, incorporate nanoparticle (NP) actuators. More specifically, Controlled drug delivery systems (DDS) have several advantages compared to the traditional forms of drugs. A drug is transported to the place of action, hence, its influence on vital tissues and undesirable side effects can be minimized. Accumulation of therapeutic compounds in the target site increases and, consequently, the required doses of drugs are lower. This modern form of therapy is especially important when there is a discrepancy between the dose or the concentration of a drug and its therapeutic results or toxic effects. Cell-specific targeting can be accomplished by attaching drugs to specially designed carriers. Various nanostructures, including liposomes, polymers, dendrimers, silicon or carbon materials, and magnetic nanoparticles, have been tested as carriers in drug delivery systems. Polymeric nanoparticles are one technology being developed to enable clinically feasible oral delivery. More specifically, the term “nanostructure” or “nanoparticle” is used herein to denote any microscopic particle smaller than about 100 nm in diameter. In some other embodiments, the carrier is an organized collection of lipids. When referring to the structure forming lipids, specifically, micellar formulations or liposomes comprising at least one of the splicing modulating agents of the disclosure. In some embodiments, the lipid may be natural, semi-synthetic or fully synthetic lipid, as well as electrically neutral, negatively or positively charged lipid. In some embodiments, the lipid may be a naturally occurring phospholipid. Examples of lipids forming glycerophospholipids include, without being limited thereto, glycerophospholipid. phosphatidylglycerols (PG) including dimyristoyl phosphatidylglycerol (DMPG); phosphatidylcholine (PC), including egg yolk phosphatidylcholine, dimyristoyl phosphatidylcholine (DMPC), 1-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS). Examples of cationic lipids may include, for example, 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy) propyl]-N,N,N-trimethylammonium chloride (DOTMA); 3β[N—(N′,N′-dimethylaminoethane) carbamoly]cholesterol (DC-Chol); and dimethyl-dioctadecylammonium (DDAB), N-[2-[[2,5-bis[3-aminopropyl)amino]-1-oxopentyl]amino]ethyl]-N,N-dimethyl-2,3-bis[(1-oxo-9-octadecenyl)oxy]-1 propanaminium (DOSPA), and ceramide carbamoyl spermine (CCS), or the neutral lipid dioleoylphosphatidyl ethanolamine (DOPE) derivatized with polylysine to form a cationic lipopolymer.

In some embodiments, the structure forming lipids may be combined with other lipids, such as a sterol. Sterols and in particular cholesterol are known to have an effect on the properties of the lipid's organized structure (lipid assembly), and may be used for stabilization, for affecting surface charge, membrane fluidity. In some embodiments, a sterol, e.g. cholesterol is employed in order to control fluidity of the lipid structure. The greater the ratio sterol:lipids (the structure forming lipids), the more rigid the lipid structure is.

A further aspect of the present disclosure relates to a method for treating, preventing, ameliorating, inhibiting or delaying the onset of a pathologic disorder in a mammalian subject. The methods disclosed herein comprises the step of administering to the subject an effective amount of at least one of: (a), at least one non-naïve cell of the T lineage manipulated for enhanced skipping of exon 6 of the Fas gene, or a cell population comprising at least one of the manipulated cell/s. The non-naïve manipulated cell of the T lineage used by the disclosed methods predominantly expresses sFas. In yet some further embodiments, the methods disclosed herein may further, or alternatively administer (b), at least one splicing modulating agent or any vector, vehicle, matrix, nano- or micro-particle or composition comprising the at least one agent. The splicing modulating agent used by the disclosed methods comprises at least one nucleic acid molecule that targets at least one target nucleic acid sequence participating directly or indirectly in at least one splicing event of the Fas gene. Still further, the disclosed methods may further, or alternatively administer (c) a composition comprising the non-naïve cell/s of the T lineage of (a) and/or the at least one splicing modulating agent of (b).

In some embodiments, the disclosed methods are appliable for a pathologic disorder. In some specific embodiments, such pathologic disorder may be at least one of a proliferative disorder, an inflammatory disorder, an infectious disease caused by a pathogen, an autoimmune-disease, a cardiovascular disease and/or a neurodegenerative disorder.

When referring to a proliferative disorder, it is to be understood to relate to at least one malignant neoplastic disorder, optionally the neoplastic disorder is at least one hematological malignancy, and/or at least one solid tumor.

In some specific embodiments, the subject treated by the method of the present disclosure may be a subject suffering of an immune-related disorder. An “Immune-related disorder” or “Immune-mediated disorder”, as used herein encompasses any condition that is associated with the immune system of a subject, more specifically through inhibition of the immune system, or that can be treated, prevented or ameliorated by reducing degradation of a certain component of the immune response in a subject, such as the adaptive or innate immune response. An immune-related disorder may include infectious condition (e.g., by a pathogen, specifically, viral, bacterial or fungal infections), inflammatory disease, autoimmune disorders, metabolic disorders and a proliferative disorder, specifically, cancer. In some specific embodiments wherein the immune-related disorder or condition may be a primary or a secondary immunodeficiency.

By “neoplasia” is meant any disease that is caused by or results in inappropriately high levels of cell division, inappropriately low levels of apoptosis, or both. For example, cancer is an example of a neoplasia. In yet some further embodiments, the target cell of the methods of the disclosure may be a cell of a subject suffering from at least one neoplastic disorder.

In more specific embodiments, such neoplastic disorder is cancer. In some specific embodiments, the cells, methods, compositions and any of the splicing modulating agents of the disclosure that comprise at least one nucleic acid sequence, e.g., any of the SSO/s or any of the guide RNAs and any PEN systems of the disclosure, may be used for treating cancer or any other neoplastic disorders or proliferative disorders. As used herein to describe the present disclosure, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods, compositions, kits and AON/s of the disclosure of the present disclosure may be applicable for treatment of a patient suffering from any one of non-solid and solid tumors. Malignancy, as contemplated in the present disclosure may be any one of melanoma, carcinomas, lymphomas, leukemias, myeloma and sarcomas.

In some specific and non-limiting embodiments, the present disclosure may be applicable for melanoma.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes. Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present disclosure can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The disclosure may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extrahepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

Still further, in some embodiments, the term cancer includes but is not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer). Still further the invention relates to any neurological tumor, for example, neuroblastoma, astrocytoma, CNS lymphoma, neuroma, glioma, Chordoma, medulloblastoma, Oligodendroglioma, Craniopharyngioma, and any mixed neurological tumor.

It should be understood that the present disclosure thus encompasses the treatment of any of the malignancies described in this context, specifically any malignancies described in connection with associated TAAs as described herein before in connection with other aspects of the present disclosure.

In yet some further embodiments, of the methods of the present disclosure may be also applicable for treating autoimmune disorders, that are also referred to as disorders of immune tolerance, when the immune system fails to properly distinguish between self and non-self-antigens.

Thus, according to some embodiments, the method of the present disclosure may be used for the treatment of a patient suffering from any autoimmune disorder. In some specific embodiments, the methods of the present disclosure may be used for treating an autoimmune disease such as for example, but not limited to, inflammatory bowel disease (IBD), ulcerative colitis, Crohn's disease, fatty liver disease, Lymphocytic colitis, Ischaemic colitis, Diversion colitis, Behcet's syndrome, Indeterminate colitis, rheumatoid arthritis, systemic lupus erythematosus (SLE), Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

In some further embodiments, the therapeutic methods disclosed herein may be applicable for at least one metabolic disorder. In more specific embodiments, such metabolic disorder may comprise at least one cardiovascular disorder, liver diseases, diabetes and metabolic syndrome.

Metabolic disease is any of the diseases or disorders that disrupt normal metabolism, the process of converting food to energy on a cellular level. Thousands of enzymes participating in numerous interdependent metabolic pathways carry out this process.

Metabolic diseases negatively affect the ability of the cell to perform critical biochemical reactions that involve the processing or transport of proteins (amino acids), carbohydrates (sugars and starches), or lipids (fatty acids). Numerous molecular pathways and thus, several organs, can be affected. Many of the metabolic diseases are caused by genetic mutations or by a combination of genetic and environmental factors. Some of the metabolic diseases or diseases associated with metabolic disorders include for example: diabetes (or diabetes mellitus), a group of common endocrine diseases characterized by sustained high blood sugar levels affecting nearly every major bodily organ; non-alcoholic steatohepatitis (NASH), a liver inflammation and damage caused by a buildup of fat in the liver; steatosis (or fatty change), an abnormal retention of fat (lipids) within a cell or organ, most often affects the liver, but can also occur in other organs, including the kidneys, heart, and muscle; obesity, a medical condition, in which excess body fat has accumulated to such an extent that it may negatively affect health; dyslipidemia, is the imbalance of lipids such as cholesterol, low-density lipoprotein cholesterol, (LDL-C), triglycerides, and high-density lipoprotein (HDL); cardiovascular diseases, a group of disorders of the heart and blood vessels and include coronary heart disease, cerebrovascular disease, rheumatic heart disease and other conditions.

In yet some other embodiments, the methods of the present disclosure may be also applicable for treating a subject suffering from an infectious disease. More specifically, such infectious disease may be any pathological disorder caused by a pathogen. As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts or a nematode.

In yet some further embodiments, the methods of the present disclosure may be applicable in boosting the immune response against a pathogen that may be in further specific embodiment, a viral pathogen or a virus. The term “virus” as used herein, refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. It should be noted that the term “viruses” is used in its broadest sense to include viruses of the families adenoviruses, papovaviruses, herpesviruses: simplex, varicella-zoster, Epstein-Barr (EBV), Cytomegalo virus (CMV), pox viruses: smallpox, vaccinia, hepatitis B (HBV), rhinoviruses, hepatitis A (HBA), poliovirus, respiratory syncytial virus (RSV), Middle East Respiratory Syndrome (MERS), Severe acute respiratory syndrome (SARS), rubella virus, hepatitis C (HBC), arboviruses, rabies virus, influenza viruses A and B, measles virus, mumps virus, human deficiency virus (HIV), HTLV I and II, Dengue virus and Zika virus.

In some further embodiments, the methods of the present disclosure may be applicable for immune-related disorder or condition that may be a pathologic condition caused by at least one pathogen. It should be appreciated that an infectious disease as used herein also encompasses any infectious disease caused by a pathogenic agent, specifically, a pathogen. Pathogenic agents include prokaryotic microorganisms, lower eukaryotic microorganisms, complex eukaryotic organisms, viruses, fungi, prions, parasites, yeasts, toxins and venoms. In yet some other specific embodiments, the methods and composition of the invention may be applicable for treating an infectious disease caused by bacterial pathogens. More specifically, a prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia co/i, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes. A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the invention. A complex eukaryotic organism includes worms, insects, arachnids, nematodes, aemobe, Entamoeba histolytica, Giardia lamblia, Trichomonas vagina/is, Trypanosoma brucei gambiense, Trypanosoma cruzi, Balantidium co/i, Toxoplasma gondii, Cryptosporidium or Leishmania. More specifically, in certain embodiments the methods and compositions of the invention may be suitable for treating disorders caused by fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous and possess branched somatic structures (hyphae) surrounded by cell walls containing glucan or chitin or both and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidioidoinycosis, and candidiasis.

As noted above, the present invention also provides for the methods and compositions for the treatment of a pathological disorder caused by “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species. As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

Thus, the methods of the present disclosure may offer a promising therapeutic modality for a variety of innate and acquired immunodeficiencies caused by immunosuppressive treatments (chemo- and radiotherapy), pathogenic infections, cancer and HSCT. More specifically, Immunodeficiency (or immune deficiency) is a state in which the immune system's ability to fight infectious disease and cancer is compromised or entirely absent. Most cases of immunodeficiency are acquired (“secondary”) due to extrinsic factors that affect the patient's immune system. Examples of these extrinsic factors include viral infection, specifically, HIV, extremes of age, and environmental factors, such as nutrition. In the clinical setting, the immunosuppression by some drugs, such as steroids, can be either an adverse effect or the intended purpose of the treatment. Examples of such use are in organ transplant surgery as an anti-rejection measure and in patients suffering from an overactive immune system, as in autoimmune diseases. Immunodeficiency also decreases cancer immuno-surveillance, in which the immune system scans the cells and kills neoplastic ones. Still further, Primary immunodeficiencies (PID), also termed innate immunodeficiencies, are disorders in which part of the organism immune system is missing or does not function normally. To be considered a primary immunodeficiency, the cause of the immune deficiency must not be caused by other disease, drug treatment, or environmental exposure to toxins). Most primary immune deficiencies are genetic disorders; the majority is diagnosed in children under the age of one, although milder forms may not be recognized until adulthood. While there are over 100 recognized PIDs, most are very rare. There are several types of immunodeficiency that include, Humoral immune deficiency (including B cell deficiency or dysfunction), which generally includes symptoms of hypogammaglobulinemia (decrease of one or more types of antibodies) with presentations including repeated mild respiratory infections, and/or agammaglobulinemia (lack of all or most antibody production) and results in frequent severe infections (mostly fatal); T cell deficiency, often causes secondary disorders such as acquired immune deficiency syndrome (AIDS); Granulocyte deficiency, including decreased numbers of granulocytes (called as granulocytopenia or, if absent, agranulocytosis) such as of neutrophil granulocytes (termed neutropenia); granulocyte deficiencies also include decreased function of individual granulocytes, such as in chronic granulomatous disease; Asplenia, where there is no function of the spleen; and Complement deficiency in which the function of the complement system is deficient. Secondary immunodeficiencies occur when the immune system is compromised due to environmental factors. Such factors include but are not limited to radiotherapy as well as chemotherapy. While often used as fundamental anti-cancer treatments, these modalities are known to suppress immune function, leaving patients with an increased risk of infection; indeed, infections were found to be a leading cause of patient death during cancer treatment. Neutropenia was specifically associated with vulnerability to life-threatening infections following chemotherapy and radiotherapy. In more specific embodiments, such secondary immunodeficiency may be caused by at least one of chemotherapy, radiotherapy, biological therapy, bone marrow transplantation, gene therapy, adoptive cell transfer or any combinations thereof.

It should be understood that the cells of the T lineage used in the disclosed methods are any of the cells as defined and disclosed by the present disclosure. In some further embodiments, the splicing modulating agent used in the disclosed methods may be any one of the splicing modulating agents (e.g., the SSOs, and/or the gRNA) defined and disclosed by the present disclosure.

As indicated herein, the disclosure provides therapeutic methods for treating the specific conditions or diseases disclosed herein before. As used herein, “disease”, “disorder”, “condition” and the like, as they relate to a subject's health, are used interchangeably and have meanings ascribed to each and all of such terms. It should be appreciated that the disclosure provides therapeutic methods applicable for any of the disorders disclosed above, as well as to any condition or disease associated therewith. It is understood that the interchangeably used terms “associated”, “linked” and “related”, when referring to pathologies herein, mean diseases, disorders, conditions, or any pathologies which at least one of share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

In some embodiments, the therapeutic methods described herein may use an effective amount of the splicing modulating agent of the disclosure (SSO/s and gRNAs), particularly for therapeutic purposes. The terms “effective amount” or “sufficient amount” used by the methods of the disclosure, mean an amount necessary to achieve a selected result. More specifically, the amount of the specific modulating agent that is sufficient to induce skipping of exon 6 of Fas. Moreover, such effective amount is sufficient to induce and enhance skipping of exon 6 of Fas, thereby leading to increased expression of sFas and/or reduced expression of mFas.

The “effective treatment amount” is determined by the severity of the disease in conjunction with the preventive or therapeutic objectives, the route of administration and the patient's general condition (age, sex, weight and other considerations known to the attending physician). The SSO/s or gRNA concentration can range between 0.1 mg/kg and 100 mg/kg but other concentrations may apply. More specifically, in certain embodiments, the dose for systemic administration is from 0.1 mg/kg to 500 mg/kg. In certain embodiments, the dose for systemic administration is from 0.1 mg/kg to 100 mg/kg, 0.5 mg/kg to 100 mg/kg, 1 mg/kg to 100 mg/kg, 1 mg/kg to 50 mg/kg, 1 mg/kg to 25 mg/kg. In some embodiments, the dose for systemic administration is from 5 mg/kg to 50 mg/kg. In yet some further embodiments, the dose for systemic administration is from 0.1 mg/kg to 25 mg/kg, 0.1 mg/kg to 10 mg/kg, 1 mg/kg to 10 mg/kg or from 1 mg/kg to 5 mg/kg.

The terms “treat, treating, treatment” as used herein and in the claims mean ameliorating one or more clinical indicia of disease activity by administering a pharmaceutical composition of the disclosure in a patient having a pathologic disorder.

More specifically, the term “treatment”, as used herein refers to the administering of a therapeutic amount of the cells, splicing modulator, or composition of the present disclosure which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “amelioration” as referred to herein, relates to a decrease in the symptoms, and improvement in a subject's condition brought about by the cells, splicing modulator, compositions and methods according to the disclosure, wherein said improvement may be manifested in the forms of inhibition of pathologic processes associated with cancer disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the disclosure described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the disclosure.

Still further, as mentioned above, the term “treatment or prevention” as used herein, refers to the complete range of therapeutically positive effects of administrating to a subject including inhibition, reduction of, alleviation of, and relief from, a cancer and illness, a cancer symptoms or undesired side effects of a cancer. More specifically, treatment or prevention of relapse, or re recurrence of the disease, includes the prevention or postponement of development of the disease, prevention or postponement of development of symptoms and/or a reduction in the severity of such symptoms that will or are expected to develop. These further include ameliorating existing symptoms, preventing—additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% or even 100%.

With regards to the above, it is to be understood that, where provided, percentage values such as, for example, 10%, 50%, 100%, 120%, 500%, etc., are interchangeable with “fold change” values, i.e., 0.1, 0.5, 1.2, 5, etc., respectively.

The present disclosure relates to the treatment of subjects or patients in need thereof. By “patient” or “subject in need” it is meant any organism who may be affected by the above-mentioned conditions, and to whom the therapeutic methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the subject may be also any reptile or zoo animal. More specifically, the methods of the present disclosure are intended for mammals. By “mammalian subject” is meant any mammal for which the proposed therapy is desired, including human, livestock, equine, canine, and feline subjects, most specifically humans.

In yet some further aspect, the disclosure provides therapeutic effective amount of at least one splicing modulating agent comprising at least one nucleic acid sequence or of any vector, vehicle, matrix, nano- or micro-particle or composition comprising the at least one agent, for use in a method for treating, inhibiting, preventing, ameliorating or delaying the onset of at least one neoplastic disorder in a subject. It should be noted that the at least one nucleic acid sequence of the agent targets at least one target nucleic acid sequence that participates directly or indirectly in at least one splicing event of at least one target gene. The introduction of the agent into the target cell in the treated subject induces at least one aberrant splicing event via the target nucleic acid sequence, thereby leading to enhanced skipping of exon 6 of Fas.

In some embodiments of the disclosed therapeutic methods and uses, the at least one target nucleic acid sequence that participates directly or indirectly in at least one splicing event used by the disclosed therapeutic methods comprises at least one of: a splicing junction, a splice donor site, a splice acceptor site, an exonic splicing enhancer, splicing silencer, an intronic splicing enhancer and an intronic splicing silencer of the target gene.

In some embodiments of the disclosed therapeutic methods and uses, such target nucleic acid sequence is in the Fas gene, as specified above, more specifically in exon 6 of Fas gene. In some embodiments the target nucleic acid sequence is comprised within and/or comprises at least one of 5′SS of exon 6, 3′SS of exon 6, exon 6, at least one intron located upstream and/or downstream to said exon 6, and/or at least one splicing junction flanking the exon 6 of Fas gene.

In yet some specific embodiments of the disclosed therapeutic methods and uses, the target nucleic acid sequence is comprised within intron 5 and exon 6 (3′ splice site) and/or exon 6 and intron 6 (5′ splice site) of the Fas gene.

In some embodiments of the disclosed therapeutic methods and uses, the splicing modulating agent used by the disclosed therapeutic methods comprises at least one of: (a), at least one oligonucleotide comprising a nucleic acid sequence complementary to at least part of the target nucleic acid sequence; In yet some further embodiments, the splicing modulating agent used by the disclosed therapeutic methods may further, or alternatively comprises (b), at least one nucleic acid sequence comprising at least one gRNA that targets at least one protospacer within the target nucleic acid sequence, or any nucleic acid sequence encoding the gRNA. Such gRNAs may guide at least one PEN to the target nucleic acid sequence in the Fas gene.

In some embodiments of the disclosed therapeutic methods and uses, the splicing modulating agent used by the disclosed therapeutic methods comprises at least one oligonucleotide. Such oligonucleotide may be ASO, and/or SSO, which comprises at least fifteen contiguous nucleobases complementary, or that display complementarity to at least part of the at least one nucleic acid sequence that participates directly or indirectly in at least one splicing event.

In some embodiments of the disclosed therapeutic methods and uses, the SSO and/or ASO used by the disclosed therapeutic methods comprise nucleic acid sequence complementary to intron 5/exon 6 and/or to exon 6/intron 6 of the Fas gene.

In some embodiments of the disclosed therapeutic methods and uses, the ASO and/or SSO used by the disclosed therapeutic methods is chemically modified.

Still further in some embodiments of the disclosed therapeutic methods and uses, the SSO used in the disclosed therapeutic methods may comprise the nucleic acid sequence as denoted by any one of SEQ ID NO: 2, 4, 6, 8, 10 and 12 or any variants and/or derivatives thereof. It should be understood that the disclosed methods and uses may be applicable for any of the SSOs disclosed by the present disclosure.

According to some embodiments of the disclosed therapeutic methods and uses, the PEN used in the therapeutic methods comprises at least one CRISPR/cas protein. The splicing modulating agent of the disclosed methods comprises:
(a) at least one nucleic acid sequence comprising at least one gRNA, or any nucleic acid sequence encoding said gRNA; or any kit, composition, vector or vehicle comprising said gRNA or nucleic acid sequence encoding the gRNA; Optionally, the splicing modulating agent may further comprise (b) at least one CRISPR/cas protein, or any nucleic acid molecule encoding the Cas protein, or any kit, composition, vector or vehicle comprising said CRISPR/cas protein or nucleic acid sequence encoding said CRISPR/cas protein.

Still further, the gRNA used in the disclosed methods targets at least one protospacer within at least one of the 5′SS of exon 6 and/or the 3SS of exon 6 of Fas gene.

In some specific embodiments, the gRNA that may be used in the disclosed methods may comprise the nucleic acid sequence as denoted by SEQ ID NO: 15, or any variants, homologs or derivatives thereof In some embodiments of the disclosed therapeutic methods and uses, the non-naïve manipulated cell of the T lineage used by the disclosed therapeutic methods, express reduced levels of membrane mFas.

In yet some other embodiments of the disclosed therapeutic methods and uses, the non-naïve manipulated cell of the T lineage used by the disclosed therapeutic methods is further engineered to express at least one receptor molecule. Such at least one receptor molecule comprises at least one target binding domain specific against at least one target antigen.

In some embodiments of the disclosed therapeutic methods and uses, the at least one receptor molecule is at least one of: (a) a TCR molecule specific for at least one target antigen; and/or (b) a CAR molecule specific for at least one target antigen.

In some embodiments of the disclosed therapeutic methods and uses, the at least one target antigen targeted by the TCR or CAR molecules further expressed by the non-naïve cell of the T lineage of the present disclosure, may be at least one of: at least one tumor associated antigen (TAA), at least one tumor specific antigen (TSA), at least one neoantigen, at least one viral antigen, at least one bacterial antigen, at least one fungal antigen and/or at least one parasite antigen, as described herein before in connection with other aspects of the present disclosure.

Still further, in some embodiments of the disclosed therapeutic methods and uses, the at least one non-naïve cell of the T lineage manipulated for enhanced skipping of exon 6 of the Fas gene used by the disclosed therapeutic methods is characterized by at least one of: (i) reduced expression of mFas; (ii), increased cytokine secretion; (iii) increased expression of activation markers; (iv) increased cell survival and/or viability; (v) increased cytotoxicity; and/or (vi) reduced expression of exhaustion markers. In yet some further embodiments the non-naïve cell of the T lineage manipulated for enhanced skipping of exon 6 of the Fas according to the present disclosure may be characterized by increased mitochondrial production and/or increased resistance to FASL, increase or decreased CD4:CD8 ratios, and anti-tumor immunity.

It should be understood that in some embodiments, the indicated results of the cell manipulation for enhanced skipping of exon 6 of the Fas gene used by the disclosed therapeutic methods may be in response to a specific stimulation in an in vivo and/or in vitro ex vivo setting, for example, activation of T-cells (e.g., by anti-CD3 antibodies and the like).

Still further, it should be understood that the present disclosure encompasses therapeutic methods that are based either on ex vivo manipulation of the cells of the T lineage to enhanced skipping of exon 6 of the Fas gene according to the present disclosure. Alternatively, or additionally, on in vivo methods that are based on in vivo manipulation of the cells of the T lineage within the body of the subject, wherein the subject is administered with an effective amount of the splicing modulating agents as disclosed herein.

More specifically, in some embodiments, as the ex vivo therapeutic approach, the subject administered in accordance with the disclosed therapeutic methods with the at least one non-naïve cell of the T lineage that were manipulated ex vivo or in vitro for enhanced skipping of exon 6 of the Fas gene. In some embodiments, such ex vivo/in vitro manipulated cells may be of an autologous or allogeneic source.

As indicated herein, the present disclosure provides method allowing in vivo as well as ex-vivo or in vitro manipulation of non-naïve cell of the T lineage. Thus, in some embodiments, in case the manipulation of the cells is performed ex vivo or in vitro (with cells of allogeneic or autologous source), the engineered cells are transferred back to the subject, by adoptive transfer. In such case, the method comprising the step of administering to the subject (a), the manipulated cells or any compositions thereof.

The term “adoptive transfer” as herein defined applies to all the therapies that consist of the transfer of components of the immune system, specifically cells that are already capable of mounting a specific immune response. In such option, the insertion of the at least one nucleic acid molecule that targets at least one target nucleic acid sequence participating directly or indirectly in at least one splicing event of the Fas gene and optionally, the at least one receptor disclosed herein, is performed in cells of an autologous or allogeneic source, that are then administered to the subject, specifically, by adoptive transfer. Still further, in some embodiments cells of a xenogeneic source may be also used.

In some embodiments, the cells are manipulated ex vivo or in vitro by contacting the cells with an effective amount of the splicing modulating agent, for example, an agent comprising the indicated nucleic acid molecules (e.g., SSOs and/or gRNAs) that target at least one target nucleic acid sequence participating directly or indirectly in at least one splicing event of the Fas gene, may be cells of an autologous source.

As indicated above, the ex vivo or in vitro manipulated cells may be in some embodiments, of an autologous or of an allogeneic source. The term “autologous” when relating to the source of cells, refers to cells derived or transferred from the same subject that is to be treated by the methods of the invention. The term “allogenic” when relating to the source of cells, refers to cells derived or transferred from a different subject, referred to herein as a donor, of the same species.

In some alternative or additional embodiments, the cells of the T lineage in accordance with the present disclosure may be manipulated in vivo. According to such embodiments the splicing modulating agent or any vector, vehicle, matrix, nano- or micro-particle or composition comprising the at least one agent is administered to the subject treated by the disclosed therapeutic methods. Such splicing modulating agent comprises at least one of (a) at least one oligonucleotide comprising a nucleic acid sequence complementary to at least part of the target nucleic acid sequence; and (b) at least one nucleic acid sequence comprising at least one gRNA that targets at least one protospacer within the target nucleic acid sequence, or any nucleic acid sequence encoding the gRNA. Such gRNAs may guide at least one PEN to the target nucleic acid sequence.

A further aspect of the present disclosure relates to a therapeutically effective amount of at least one of (a) at least one non-naïve cell of the T lineage manipulated for enhanced skipping of exon 6 of the Fas gene, or a cell population comprising at least one of the cell/s. It should be noted that the non-naïve manipulated cell of the T lineage predominantly expresses sFas. Still further, in some embodiments of the disclosed use, the cell/s optionally further expresses at least one receptor molecule.
(b) at least one splicing modulating agent or any vector, vehicle, matrix, nano- or micro-particle or composition comprising the at least one agent. Such splicing modulating agent comprises at least one nucleic acid molecule that targets at least one target nucleic acid sequence participating directly or indirectly in at least one splicing event of the Fas gene; and/or
(c) a composition comprising the cell/s of (a) and/or the at least one splicing modulating agent of (b), for use in a method for treating, preventing, ameliorating, inhibiting or delaying the onset of a pathologic disorder in a mammalian subject.

In some embodiments of the disclosed effective amount for use, the non-naïve cell of the T lineage manipulated for enhanced skipping of exon 6 of the Fas gene or a cell population comprising at least one of the cell/s used herein, may be any of the cells as defined by the present disclosure, and/or any of the compositions as defined by the present disclosure.

In some specific embodiments of the disclosed therapeutic methods and uses, the cells are activated prior to manipulation of the exon skipping, e.g., prior to contacting the cells with the splicing modulating agents (e.g., SSOs, gRNAs) as discussed herein. Activation may include but is not limited to contacting the cells with an anti-CD3 antibody and/or with anti-CD28 antibody, and/or with any specific and non-specific activation component. Thus, in some embodiments, the disclosed methods may further comprise the step of activation of the cells. In some embodiments, such activation step is performed before manipulation of the splicing, and specifically, the skipping of exon 6 is performed. Thus, the modulation on some embodiments is performed on activated cells of the T lineage. In accordance with in vivo therapeutic uses, the disclosed method may in some embodiments perform the activation step either before, during or after the modulation of the splicing event using the splicing modulating agent. The step of activation may comprise contacting the cells with an effective amount of an activating compound (e.g., anti-CD3 antibody and/or with anti-CD28 antibody). Still further, in case the contacting step is performed in vivo in a subject, prior to administration of the splicing modulating agent, the disclosed method comprises administering to the treated subject an effective amount of the activating agent, e.g., anti-CD3 antibody and/or with anti-CD28 antibody.

A further aspect provided by the present disclosure relates to a method for improving activity and/or survival of at least one cell of the T lineage. The method disclosed herein may comprise the step of contacting at least one cell with an effective amount of at least one splicing modulating agent comprising at least one nucleic acid molecule or any vector, vehicle, matrix, nano- or micro-particle or composition comprising the at least one agent. The at least one nucleic acid molecule used by the disclosed methods targets at least one target nucleic acid sequence that participates directly or indirectly in at least one splicing event of the Fas gene, thereby enhancing skipping of exon 6. In some embodiments, the at least one target nucleic acid sequence that participates directly or indirectly in at least one splicing event of the Fas gene, as used in the disclosed methods comprises at least one of a splicing junction, a splice donor site, a splice acceptor site, an exonic splicing enhancer, splicing silencer, an intronic splicing enhancer and an intronic splicing silencer of the target gene.

In yet some further embodiments, the target nucleic acid sequence is comprised within and/or comprises at least one of 5′SS of exon 6, 3′SS of exon 6, exon 6, at least one intron located upstream and/or downstream to said exon 6, and/or at least one splicing junction flanking said exon 6.

In some embodiments, the target nucleic acid sequence is comprised within intron 5 and exon 6 and/or exon 6 and intron 6 of the Fas gene.

Still further, the splicing modulating agent used in the disclosed methods comprises at least one of
(a) at least one oligonucleotide comprising a nucleic acid sequence complementary to at least part of the target nucleic acid sequence; and
(b) at least one nucleic acid sequence comprising at least one gRNA that targets at least one protospacer within the target nucleic acid sequence, or any nucleic acid sequence encoding said gRNA. Such gRNA guides at least one PEN to said target nucleic acid sequence.

In some embodiments, the splicing modulating agent used in the disclosed methods comprises at least one oligonucleotide. The oligonucleotide may be ASO, and/or SSO.

In some embodiments, the oligonucleotides comprise at least fifteen contiguous nucleobases that display complementarity to at least part of the at least one nucleic acid sequence that participates directly or indirectly in at least one splicing event.

In some embodiments, the SSO and/or ASO comprise nucleic acid sequence complementary to intron 5/exon 6 and/or to exon 6/intron 6 of the Fas gene.

In some embodiments, the ASO and/or SSO may be chemically modified.

In some embodiments, the SSO used in the disclosed method for modulating the cell of the present disclosure may comprise the nucleic acid sequence as denoted by SEQ ID NO: 2, 4, 6, 8, 10 and 12 or any variants and/or derivatives thereof.

In some embodiments, the PEN used in the disclosed method comprises at least one CRISPR/cas protein. In some embodiments the splicing modulating agent used in the disclosed method comprises:
(a) at least one nucleic acid sequence comprising at least one gRNA, or any nucleic acid sequence encoding the gRNA; or any kit, composition, vector or vehicle comprising the gRNA or nucleic acid sequence encoding the gRNA. The splicing modulating agent used in the disclosed method may further optionally comprises(b) at least one CRISPR/cas protein, or any nucleic acid molecule encoding the Cas protein, or any kit, composition, vector or vehicle comprising the CRISPR/cas protein or nucleic acid sequence encoding the CRISPR/cas protein.

In some embodiments, the gRNA used by the disclosed method, targets at least one protospacer within at least one of the 5′SS of exon 6 and/or the 3SS of exon 6 of Fas gene.

In some embodiments, the manipulated non-naïve cell of the T lineage used by the disclosed method express reduced levels of mFas.

Still further, in some embodiments, the non-naïve cell of the T lineage used by the disclosed method is further engineered to express at least one receptor molecule. Such receptor molecule comprises at least one target binding domain specific against at least one target antigen.

In some embodiments, the disclosed method for improving activity and/or survival of at least one non-naïve cell of the T lineage results in an improved cell characterized by at least one of the following properties: (i) reduced expression of mFas; (ii), increased cytokine secretion; (iii) increased expression of activation markers; (iv) increased cell survival; (v) increased cytotoxicity; and/or (vi) reduced expression of exhaustion markers; and increased production of the soluble form of Fas. In yet some further embodiments the non-naïve cell of the T lineage manipulated for enhanced skipping of exon 6 of the Fas according to the present disclosure may be characterized by increased mitochondrial production and/or increased resistance to FASL, increase or decreased CD4:CD8 ratios and increase tumor immunity.

In yet some other embodiments, the step of contacting the non-naïve cell/s of the T lineage with at least one splicing modulating agent, or any vector, vehicle, matrix, nano- or micro-particle or composition comprising the at least one agent, is performed by the disclosed method in vivo, in vitro or ex vivo.

In yet some further embodiments, the step of contacting the non-naïve cell/s of the T lineage with the at least one splicing modulating agent may be performed in vivo in a subject suffering from at least one pathologic disorder. In specific embodiment, the contacting step comprises administering to the subject an effective amount of the at least one splicing modulating agent or with any vector, vehicle, matrix, nano- or micro-particle or composition comprising the at least one agent.

In some embodiments, the at least one pathologic disorder is at least one of: a proliferative disorder, an inflammatory disorder, an infectious disease caused by a pathogen, an autoimmune-disease, a cardiovascular disease and/or a neurodegenerative disorder.

Still further, in some embodiments, the contacting step of the cell/s with at least one splicing modulating agent may be performed by the disclosed method in vitro or ex vivo, thereby obtaining a non-naïve cell of the T lineage manipulated for enhanced skipping of exon 6 of the Fas gene, or a cell population comprising at least one of the cell. The non-naïve manipulated cell of the T lineage produced by the disclosed method predominantly expresses sFas.

In some embodiments the methods for increasing the survival of the cells is performed ex vivo/in vitro, and is used to improve the function and quality of the T lymphocyte.

In some embodiments, the cells may be of autologous or allogeneic source.

In yet some further embodiments of the disclosed methods, the cell/s produced are as defined by the present disclosure.

In some embodiments, the disclosed methods may further comprise the step of activation of the cells. In some embodiments, such activation step is performed before manipulation of the splicing, and specifically, the skipping of exon 6 is performed. Thus, the modulation on some embodiments is performed on activated cells of the T lineage. In accordance with in vivo therapeutic uses, the disclosed method may in some embodiments perform the activation step either before, during or after the modulation of the splicing event using the splicing modulating agent.

A further aspect of the present disclosure relates to a method of enhancing expression of soluble Fas (sFas) and/or inhibiting expression of membrane FAS (mFas) in a cell of the T lineage. Such method comprises the step of contacting the cell with at least one splicing modulating agent comprising at least one nucleic acid molecule or any vector, vehicle, matrix, nano- or micro-particle or composition comprising the at least one agent. In some embodiments, the at least one nucleic acid molecule used by the disclosed method targets at least one target nucleic acid sequence that participates directly or indirectly in at least one splicing event of the Fas gene.

In some embodiments the disclosed method leads to inhibition of the expression of mFas by at least 50%, 60%, 70%, 80%, 90%, or 95% as compared to expression of mFas in a suitable control, e.g., in the absence of the at least one nucleic acid molecule used by the disclosed method to target at least one target nucleic acid sequence that participates directly or indirectly in at least one splicing event of the Fas gene.

In some embodiments the disclosed method leads to enhancement of the expression of sFas by at least 50%, 60%, 70%, 80%, 90%, or 95% as compared to expression of sFas in a suitable control, e.g., in the absence of the at least one nucleic acid molecule used by the disclosed method to target at least one target nucleic acid sequence that participates directly or indirectly in at least one splicing event of the Fas gene.

A further aspect of the present disclosure relates to a splicing modulating agent comprising at least one nucleic acid molecule or any vector, vehicle, matrix, nano- or micro-particle or composition comprising the at least one agent. Such at least one nucleic acid molecule targets at least one target nucleic acid sequence that participates directly or indirectly in at least one splicing event of Fas gene.

In some embodiments, the target nucleic acid sequence of the disclosed at least one splicing modulating agent is comprised within and/or comprises at least one of 5′SS of exon 6, 3′SS of exon 6, exon 6, at least one intron located upstream and/or downstream to said exon 6, and/or at least one splicing junction flanking the exon 6.

In some specific embodiments, the target nucleic acid sequence is comprised within intron 5 and exon 6 and/or exon 6 and intron 6 of the Fas gene.

Still further, the splicing modulating agent according to the present disclosure comprises at least one of:
(a) at least one oligonucleotide comprising a nucleic acid sequence complementary to at least part of the target nucleic acid sequence; and
(b) at least one nucleic acid sequence comprising at least one gRNA that targets at least one protospacer within the target nucleic acid sequence, or any nucleic acid sequence encoding said gRNA. Such gRNA guides at least one PEN to the target nucleic acid sequence in said Fas gene.

In some embodiments, the splicing modulating agent according to the present disclosure comprises at least one oligonucleotide. The oligonucleotide may be ASO, and/or SSO. In some embodiments the oligonucleotide comprises at least fifteen contiguous nucleobases complementary to at least part of the at least one nucleic acid sequence that participates directly or indirectly in at least one splicing event.

In some other embodiments, according to the present disclosure the PEN comprises at least one CRISPR/cas protein, and the splicing modulating agent comprises: (a), at least one nucleic acid sequence comprising at least one gRNA, or any nucleic acid sequence encoding said gRNA; or any kit, composition, vector or vehicle comprising the gRNA or nucleic acid sequence encoding the gRNA. The splicing modulating agent according to the present disclosure may optionally further comprises (b), at least one CRISPR/cas protein, or any nucleic acid molecule encoding said Cas protein, or any kit, composition, vector or vehicle comprising the CRISPR/cas protein or nucleic acid sequence encoding the CRISPR/cas protein.

In some yet other embodiments, the disclosed splicing modulating agent comprises:
(a) at least one SSO comprising the nucleic acid sequence as denoted by any one of SEQ ID NO: 2, 4, 6, 8, 10 and/or 12, or any variants and derivatives thereof, In some other embodiments the disclosed splicing modulating agent may further or alternatively comprises
(b) at least one gRNA comprising the nucleic acid sequence as denoted by SEQ ID NO:15, or any variants and derivatives thereof.

Still further, in some embodiments, the SSOs and ASOs, as well as the gRNAs and CRISPR, as defined by any of the definitions as disclosed by the present disclosure. be All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the disclosure are described in detail, it is to be understood that this disclosure is not limited to particular methods, and experimental conditions described, as such methods and conditions may vary. It is also to be understood that the terminology used herein is for the purpose of describing particular embodiments only, and is not intended to be limiting, since the scope of the present disclosure will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present disclosure. It should be appreciated that while these techniques are exemplary of preferred embodiments for the practice of the disclosure, those of skill in the art, in light of the present disclosure, will recognize that numerous modifications can be made without departing from the spirit and intended scope of the disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub combination or as suitable in any other described embodiment of the disclosure. Certain features described in the context of various embodiments are not to be considered essential features of those embodiments, unless the embodiment is inoperative without those elements.

Various embodiments and aspects of the present disclosure as delineated hereinabove and as claimed in the claims section below find experimental support in the following examples.

Disclosed and described, it is to be understood that this disclosure is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present disclosure will be limited only by the appended claims and equivalents thereof.

It must be noted that, as used in this specification and the appended claims, the singular forms “a”, “an” and “the” include plural referents unless the content clearly dictates otherwise.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Mice

C57BL/6 mice were purchased from Harlan Laboratories. Pmel-1 mice carry a rearranged T-cell receptor (TCR) Vbeta13, specific for the 9-mer epitope 25-32 from murine Pmel 17, the homolog of human gp100.

Cas9 Mice carry gene coding to the Cas9 protein. Pmel-1 and Cas9 mice are self-breeding.

### Reagents

PMA (Sigma-Aldrich, catalog no. P1585).

ionomycin (Sigma-Aldrich, catalog no. I0634).

Monensin Solution (1,000×) (biolegend 420701).

Brefeldin A Solution (1,000×) (biolegend 420601).

### Cells

JURKAT cells Clone E6-1 TIB-152™ (ATCC).
HEK293 CRL-1573™ (ATCC).

Cell line 624mel (HLA-A2-/MART-1+gp100)—a gift from M. Parkhurst (Surgery Branch, NCI, NIH, Bethesda. MD, 2004),
Mouse melanoma B16-F10/mhgp100 cells (B16-F10 melanoma cells transduced with pMSGV1 retrovirus, which encodes a chimeric mouse gp100 with the human gp10025-33 sequence) were a kind gift from Ken-ichi Hanada, Surgery Branch, NCI, NIH.

### Plasmids

lentiGuide-Puro vector (addgene 52963).
lentiCas9 plasmid (addgene, 52962).
pMSCV-U6sgRNA(BbsI)-PGKpuro2ABFP (addgene, 102796)

### Kits, Systems and Enzymes

CD4+ T cells (Cat. No: 130-096-533).

CD8+ T cells (Cat. No: 130-096-495).

naïve CD4+ T cells (Cat. No: 130-094-131).

Invitrogen CELLection™ Pan Mouse IgG Kit (Cat. No.: 11531D).

qScript reverse transcriptase (Quanta-bio. Cat. No.: 95047-100).

ViiA™ 7 Real-Time PCR System (Applied Biosystems).

Amaxa Nucleofector™ II System (Lonza, Basel, Switzerland).

Amaxa Human T Cell Nucleofector kit (VPA-1002).

PCRBIO HS Taq Mix Red (PCR Biosystems PB10.23-02).

DNA Clean & Concentrator-5 (Zymo research D4013).

QIAGENE DNeasy Blood & Tissue Kit (69504).

QIAGENE RNeasy plus mini kit (74134).

Human Fas/TNFRSF6/CD95 DuoSet ELISA Catalog #: DY326.

Human IFN-gamma DuoSet ELISA Catalog #: DY285B.

Human Granzyme B DuoSet ELISA Catalog #: DY2906-05.

Beckman Coulter FACS Gallios, USA, C09745.

EasySep™ Mouse CD8+ T Cell Isolation Kit (STEMCELL 19853).

ELISA MAX™ Deluxe Set Mouse IFN-γ (Biolegend 430804).

Alt-R™ S.p. Cas9 Nuclease V3 (IDT 1081059)

### Antibodies

CD3 mAb (biolegend, 317326).

CD28 mAb (biolegend, 302934).

Purified anti-mouse CD3 Antibody (Biolegend 100201).

Ultra-LEAF™ Purified anti-mouse CD28 Antibody (Biolegend 102115).

Brilliant Violet 421™ anti-human CD95 (Fas) Antibody (biolegend, 305624).

Anti-Human CD95 Monoclonal Antibody (APC Conjugated)[DX2](elabscience E-AB-F1168E).

PE anti-human CD95 (Fas) Antibody (Biolegend 305608).

APC/Fire™ 750 anti-human CD25 (biolegend, 302642).

APC anti-human CD137 (4-1BB) Antibody (biolegend, 309810).

FITC anti-human CD154 Antibody (biolegend, 310804).

PE anti-human CD8 (biolegend 980902).

Annexin V (Invitrogen™, A35110).

CFSE (5(6)-CFDA, SE) (Invitrogen™, C1157).

Zombie Aqua™ Fixable Viability Kit (Biolegend 423102).

Brilliant Violet 421™ anti-human CD69 Antibody (Biolegend 310930).

Alexa Fluor® 647 anti-human IFN-γ Antibody (Biolegend 502516).

APC/Fire™ 750 anti-human CD8 Antibody (Biolegend 344746).

Brilliant Violet 421™ anti-human CD107a (LAMP-1) Antibody (Biolegend 328626).

PE anti-mouse CD95 (Fas) Antibody (Biolegend 152608).

### Experimental Procedures

### Cell Culture and Isolation

Human PBMCs from healthy donors (HD) were separated from buffy coats by Ficoll-Paque gradient centrifugation. The cells were thawed and plated at 1E6 cells/ml concentration and cultured in complete medium (CM) at 37° C. in a humidified 5% CO2 incubator in the presence of 300 IU/ml IL-2 given every 2-3 days.

Individual cell populations were isolated from PBMCs using Miltenyi MACs magnetic isolation kits. Commercial standardized kits based on negative selection depleting non-target cells were used for the enrichment of CD4+ T cells, CD8+ T cells and naïve CD4+ T cells.

The Jurkat cell line was purchased from ATCC. The cells were cultured in RPMI1640 supplemented with 10% heat-inactivated FCS, 2 mmol/L 1-glutamine, and combined antibiotics. The line was regularly tested and was Mycoplasma free. The cells are authenticated once a year using flow cytometry markers.

### Cell Activation

PBMCs were stimulated with either 1 μg/ml purified plate-bound CD3 mAb and 1 μg/ml purified CD28 mAb. Repeated restimulations were done in similar protocol, with intervals as indicated.

Beads-based activation was done by Invitrogen CELLection™ Pan Mouse IgG Kit. 2E7 beads were coated with 8 ug of purified CD3 mAB, CD28 mAB and IgG (2.7:5.3:2) and incubated with PBMC at a ratio of 3:1 beads:cells.

JURKAT cells were activated by 200 ng/mL PMA and 300 ng/mL ionomycin.

### RNA and DNA Extraction

Total RNA was isolated from the PBMCs or JURKAT using QIAGENE RNeasy plus mini kit. RNA samples were subjected to reverse transcription analysis using qScript reverse transcriptase according to the manufacturer's instructions.

Total DNA was isolated from the PBMCs or JURKAT using QIAGENE DNeasy Blood & Tissue Kit (69504) according to the manufacturer's instructions.

RT-PCR and Real-Time qPCR Analysis

RT-PCR was performed in the SensQuest lab cycler machine (Danyel Biotech) using PCRBIO HS Taq Mix Red according to the manufacturer's instructions, the products were then run on 1.5% agarose gel and/or sent to Sanger sequencing (Hy Laboratories Ltd). Transcripts of FAS (two splicing isoforms) were quantified by real-time PCR using the ViiA™ 7 Real-Time PCR System instrument. Results were then normalized to GUSB amplified from the same cDNA mix.

The following primers were used:
FAS-FL F: GCAACACCAAGTGCAAAGAGG, as also denoted by SEQ ID NO: 25.
FAS-FL R: TCCTTTCTCTTCACCCAAACAA, as also denoted by SEQ ID NO: 26.
sFAS F: GTGCAAAGAGGAAGTGAAGAGA, as also denoted by SEQ ID NO: 27.
sFAS R: ACCTTGGTTTTCCTTTCTGTGC, as also denoted by SEQ ID NO: 28.
GUSB F: AGAGTGGTGCTGAGGATTGG, as also denoted by SEQ ID NO: 29.
GUSB R: CCCTCATGCTCTAGCGTGTC, as also denoted by SEQ ID NO: 30.
FAS5to7 F: ATGCACACTCACCAGCAACA, as also denoted by SEQ ID NO: 52.
Total_FAS F: TGCAGAAAGCACAGAAAGG, as also denoted by SEQ ID NO: 53.
Total_FAS R: TTTATTGCCACTGTTTCAGGATTT, as also denoted by SEQ ID NO: 54.

### SSO Electroporation

SSOs were synthesized by BioSpring GmbH (Germany) with a phosphorothioate backbone and 2′-O-methyl ribose modifications in each position. The SSOs were resuspended in water and kept frozen at −20° C. 5×10⁶ cells were electroporated in each reaction. SSOs were added to the electroporation buffer at 2.5 nmol/reaction. T cells were transfected using electroporation method using the Amaxa Nucleofector™ II System. T cells were washed with phosphate-buffered saline (PBS) and electroporated using the Amaxa Human T Cell Nucleofector kit (VPA-1002) following the manufacturer's instructions with the electroporation program T-007. Electroporated T cells were then resuspended in warm complete medium supplemented with 300 U/mL IL-2.

### Lentiviral Vector Design and HEK293 Transfection

The lentiGuide-Puro vector containing Puromycin resistance gene or BFP reporter gene and gRNA was used for splice site disruption.

The gRNA lentiviral particles were generated by co-transfecting HEK293T cells using entry vectors (carrying gRNAs) along with packaging plasmids (psPAX2 and pMD2.G, addgene 12260, 12259) using Lipofectamine™ 2000 Transfection Reagent (Invitrogen™, 11668019) according to manufacturer instructions. The medium was replaced after 30 h. Lentiviral particles were collected 48 h post transfection and filtered through 0.45 μm filter (Sartorius Stedim Biotech, STS6555-FMOSK).

Lentiviral particles of LentiCas9 virus were generated similarly, using the lentiCas9 plasmid.

### Lentiviral Transduction of PBMCs and JURKAT Cell Line

Activated PBMCs were transduced 48 h post activation with lentiviral particles supplemented with 10 ug/ml polybrene (MilliporeSigma, TR-1003-G) and 300 IU/ml IL-2 (1 ml of lentiviral medium/0.5*10⁶ PBMCs). Cells were spinoculated for 1 h in 200 g. The transduction medium was replaced with CM after 16-20 h. transduced cells were selected with 2.5 ug/ml Puromycin for at least 6 days.

JURKAT cells were transduced with lentiCas9 lentivirus in the same protocol using the Cas9 plasmid mentioned above. The cells were selected with 10 ug/ml Blasticidin, and then transduced with the gRNA lentivirus. The cells were selected with puromycin in the same protocol as for PBMCs.

### Cas9 Protein Electroporation

Electroporation of recombinant Cas9 protein (Takara, 632679) was done with the same protocol as for SSOs. Usually 5-10*10⁶ T cells were electroporated in each reaction. The recombinant Cas9 was added to the electroporation buffer at 10 ug/10⁶ T cells.

### Cell Expansion

PBMCs are expanded by activation with 30 ng/mL of anti-CD3 and screened PBMCs as feeders for 12 days [Dudley. Mark E. et al., Journal of immunotherapy 26 (4), 332-342 (2003); Eisenberg, G., et al., J Immunol 190 (11): 5856-5865 (2013)]. Fresh medium containing IL2 is added on day 5 and every other day thereafter.

### Flow Cytometry Analysis

PBMCs were collected and stained with the indicated fluorescein mAbs at the indicated time points and analyzed on a flow cytometer (Beckman Coulter FACS Gallios, USA, C09745), according to the manufacturer's instructions.

### ELISA Analysis

IFNγ and Granzyme-B levels in PBMCs supernatants were measured 24 h or 48 h after each restimulation using the DuoSet ELISA kit (R & D Systems, Minneapolis, MN), according to the manufacturer instructions.

sFAS levels in JURKAT cell lines supernatants were measured after 24 h of culture in fresh medium using the DuoSet ELISA kit (R & D Systems, Minneapolis, MN), according to the manufacturer instructions.

### Proliferation and Activation Induced Cell Death (AICD) Resistance Assay

PBMCs were stained with CFSE [Quah, B. J., Warren, H. S., & Parish, C. R. (2007). Monitoring lymphocyte proliferation in vitro and in vivo with the intracellular fluorescent dye carboxyfluorescein diacetate succinimidyl ester. Nature protocols, 2(9), 2049-2056. https://doi.org/10.1038/nprot.2007.2961 and stimulated with either 1 μg/ml purified plate-bound αCD3 mAb and 1 μg/ml purified αCD28 mAb over-night. Proliferation was measured as CFSE dilution on day 1 and 4 by FACS. AICD levels were measured by staining with AnnexinV and viability dye, according to manufacturer instructions on day 1, and analyzed by FACS.

### Co-Culture and Target Survival Assay

PBMCs were cocultured with the target cell line 624mel (HLA-A2+/MART-1+gp100+) presenting the NY-ESO1 melanoma antigen in different ratios, as indicated. Repeated co-cultures were done with intervals of 48 hours. Target survival rated were measured by FACS and calculated as: Live Target/Total live cells.

### Mice Ex-Vivo Assay

Splenocytes from pmel/cas9 mice were extracted and CD8+ T cells using EasySep™ Mouse CD8+ T Cell Isolation Kit. cultured in splenocytes medium at 37° C. in a humidified 5% CO2 incubator in the presence of 100 IU/ml IL-2 given every 2-3 days. Cells were activated for 48 h using 2 ug/ml plate-bound anti-mouse CD3 and anti-mouse CD28.

Activated mouse CD8+ T cells were transduced with lentivirus coding for sgRNA similarly to PBMCs transduction. The cells were selected with 1.25 ug/ml puromycin for 6 days and analyzed for mFAS expression by FACS.

Cells were co-cultured with Mouse melanoma B16-F10/mhgp100 cells at different E:T ratios for 24 hours. IFN γ secretion levels were measured by ELISA using ELISA MAX™ Deluxe Set Mouse IFNγ (Biolegend). Target survival rated were measured by FACS and calculated as: Live Target/Total live cells.

### Example 1

### Activated T Cells Undergo FAS Alternative Splicing

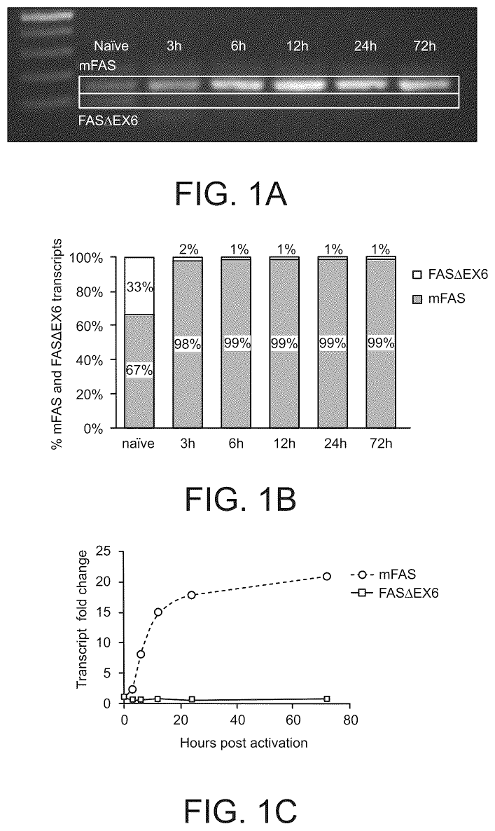

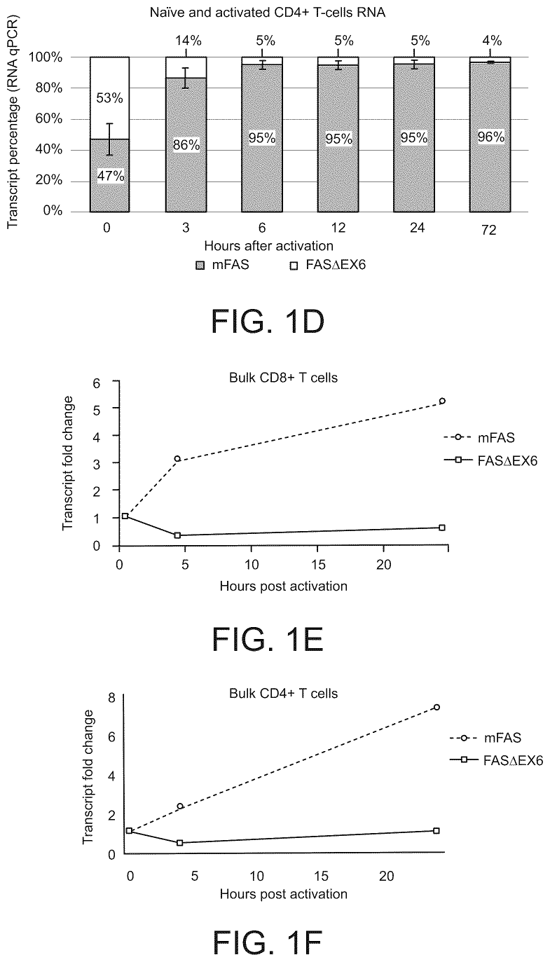

FAS alternative splicing following T-cells activation was first examined using Naïve CD4+ T cells isolated from healthy donors' PBMCs and activated with αCD3, and αCD28 antibodies coated beads. RT-PCR and agarose gel electrophoresis analysis showed that there was a change in the ratio of the membrane-bound form of FAS (mFAS) and the soluble form derived from exon 6 skipping (sFAS, also referred to herein as FASΔEX6). Naïve CD4+ T cells exhibited mFAS:FASΔEX6 ratio of about 2:1. However, three and six hours after activation, mFAS transcript was increased to 98% and 99% of the total FAS transcripts, respectively. The increased mFAS transcript was stable until 72 h after activation (FIGS. 1A, 1B).

Similar results were shown by longitudinal qPCR for FAS alternative splicing isoforms in three healthy donors PBMCs following activation. FIG. 1C shows that alongside the total FAS transcript increase, there was an increase in mFAS and no change in soluble form derived from exon 6 skipping (sFAS, FASΔEX6), turning the mFAS to the significantly dominant isoform (FIG. 1D). Naïve CD4+ T cells exhibited about 47% mFAS and 53% sFAS from the total transcript. However, three and six hours after activation, mFAS was elevated to 86% and 95% of the total FAS transcript, respectively (FIG. 1D).

In another experiment, bulk CD8+ and CD4+ T cells were isolated from PBMCs and were activated. The results show that a similar alternative splicing pattern occurs in both subpopulations (FIGS. 1E, 1F).

### Example 2

### Manipulating FAS Alternative Splicing by Exon 6 Skipping Using Splice-Switching Oligonucleotides (SSOs) System

The inventors next questioned whether manipulating FAS splicing and enhancing FAS exon 6 skipping will combine the effect of mFAS knockdown together with FASΔEX6 overexpression. FAS knockdown has already shown promising effects in T cells. The effect of FASΔEX6 overexpression is less known. The inventors next examined if the secreted FASΔEX6 has beneficial role as a decoy receptor for FASL and overexpression of FASΔEX6 may also supply environmental protection and block FAS-FASL interaction on bystander T cells.

Splice-switching oligonucleotides (SSOs) are short nucleic acid sequences with chemistry modifications, designed to manipulate mRNA splicing by interrupting the linkage of splicing factors to regulatory motives in pre-mRNA sequences.

To identify SSOs that can divert the splicing isoforms of the FAS transcript toward exon 6 skipping, six different SSOs targeting the 5′ splice site and 3′ splice site of FAS exon 6 (SSO FASΔEX6 #1 to #6, as denoted by SEQ ID NO: 2, 4, 6, 8, 10, 12, respectively) were tested and compared to null antisense oligonucleotide (ASO) (SSO SCR, SEQ ID NO: 14) (FIG. 2A and Table 1). Table 2 further indicates the FAS target sequences (SEQ ID NO: 3, 5, 7, 9, 11 and 13), and FAS coding strand as denoted by SEQ ID NO: 1. The SSOs were transfected into non-naïve, activated PBMCs from healthy donors (FIG. 2B) by electroporation and analyzed by FACS for mFAS expression (FIG. 2C) and by qPCR for alternative splicing isoforms transcripts three days after electroporation (FIGS. 2D and 2E). Some SSOs demonstrated high efficiency, leading to a 90% FASΔEX6 transcript (SSO #6), compared to 19% in SCR SSO transfected cells (FIG. 2F).

The change in splicing ratio is also reflected in the protein level, decreasing the mFAS expressing population from about 1000 in the DN SCR transfected cells to about 23 in the SS FASΔEX6 #6 transfected cells (FIG. 2G). The SSOs' effect could be seen eight days after electroporation with the significant FASΔEX6 transcript fold change (FIG. 2H). These results demonstrate that FASΔEX6 interaction with T cells membrane-bound FASL may lead to supportive reverse signaling, conducted by the FASL, which may promote immune cells' function and sustainability.

TABLE 1
FAS exon 6 skipping - SSOs system

| | | | FAS DNA coding strand | |
|---|---|---|---|---|
| | | FAS RNA | sequence | |
| SSO | SSO | target | (intron5EXON6intron6 \| | SSO |
| number | sequence | sequence | target site) | modification |
| 1 | SEQ ID | SEQ ID | SEQ ID NO: 1 | Modifications: |
| | NO: 2 | NO: 3 | aaatgtccaatgttccaacctacagGAT | 2′-MOE |
| | GGAUCCU | uguuccaaccua | CCAGATCTAACTTGGGGT | Backbone: PS |
| | GUAGGU | cagGAUCC | GGCTTTGTCTTCTTCTTTT | |
| | UGGAACA | | GCCAATTCCACTAATTGTT | |
| | | | TGGGgtaagttcttgctttgttcaaactg | |
| 2 | SEQ ID | SEQ ID | SEQ ID NO: 1 | Modifications: |
| | NO: 4 | NO: 5 | aaatgtccaatgttccaacctacagGAT | 2′-MOE |
| | GAUCUGG | caaccuacagGA | CCAGATCTAACTTGGGGTG | Backbone: PS |
| | AUCCUGU | UCCAGAUC | GCTTTGTCTTCTTCTTTTG | |
| | AGGUUG | | CCAATTCCACTAATTGTTT | |
| | | | GGGgtaagttcttgctttgttcaaactg | |
| 3 | SEQ ID | SEQ ID | SEQ ID NO: 1 | Modifications: |
| | NO: 6 | NO: 7 | aaatgtccaatgttccaacctacagGAT | 2′-MOE |
| | AGUUAG | uacagGAUCC | CCAGATCTAACTTGGGGTG | Backbone: PS |
| | AUCUGGA | AGAUCUAAC | GCTTTGTCTTCTTCTTTTG | |
| | UCCUGUA | U | CCAATTCCACTAATTGTTT | |
| | | | GGGgtaagttcttgctttgttcaaactg | |
| 4 | SEQ ID | SEQ ID | SEQ ID NO: 1 | Modifications: |
| | NO: 8 | NO: 9 | aaatgtccaatgttccaacctacagGAT | 2′-MOE |
| | CUUACCC | CACUAAUUG | CCAGATCTAACTTGGGGT | Backbone: PS |
| | CAAACAA | UUUGGGgua | GGCTTTGTCTTCTTCTTTT | |
| | UUAGUG | ag | GCCAATTCCACTAATTGTTT | |
| | | | GGGgtaagttcttgctttgttcaaactg | |
| 5 | SEQ ID | SEQ ID | SEQ ID NO: 1 | Modifications: |
| | NO: 10 | NO: 11 | aaatgtccaatgttccaacctacagGAT | 2′-MOE |
| | AAGAACU | AUUGUUUG | CCAGATCTAACTTGGGGT | Backbone: PS |
| | UACCCCA | GGguaaguucu | GGCTTTGTCTTCTTCTTTT | |
| | AACAAU | u | GCCAATTCCACTAATTGTT | |
| | | | TGGGgtaagttcttgctttgttcaaactg | |
| 6 | SEQ ID | SEQ ID | SEQ ID NO: 1 | Modifications: |
| | NO: 12 | NO: 13 | aaatgtccaatgttccaacctacagGAT | 2′-MOE |
| | AAAGCA | UUGGGguaag | CCAGATCTAACTTGGGGT | Backbone: PS |
| | AGAACU | uucuugcuuu | GGCTTTGTCTTCTTCTTTT | |
| | UACCCC | | GCCAATTCCACTAATTGTT | |
| | AA | | TGGGgtaagttcttgctttgttcaaactg | |
| SCR | SEQ ID | — | — | Modifications: |
| | NO: 14 | | | 2′-MOE |
| | ACACCAA | | | Backbone: PS |
| | CUAAUAC | | | |
| | GAAUAC | | | |

### Example 3

### Manipulating FAS Alternative Splicing by Exon 6 Skipping Induced by CRISPR/Cas9 Splice Site Disruption System

CRISPR/Cas9 system can also be used to target the 3′ SS of FAS exon 6, leading to double-strand breaking, non-homologous end joining, and disruption of the SS sequence. Therefore, the spliceosome is misidentifying the SS and skips over the exon.

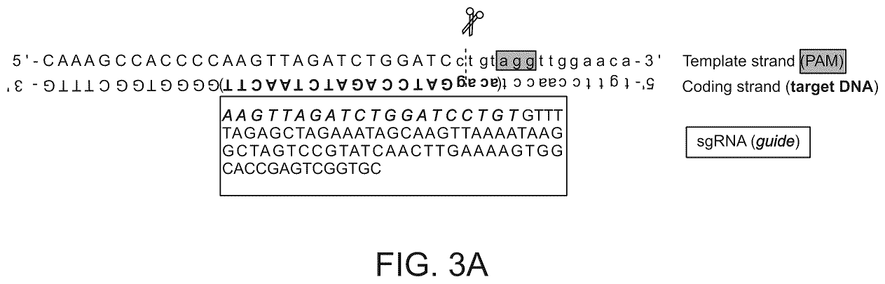

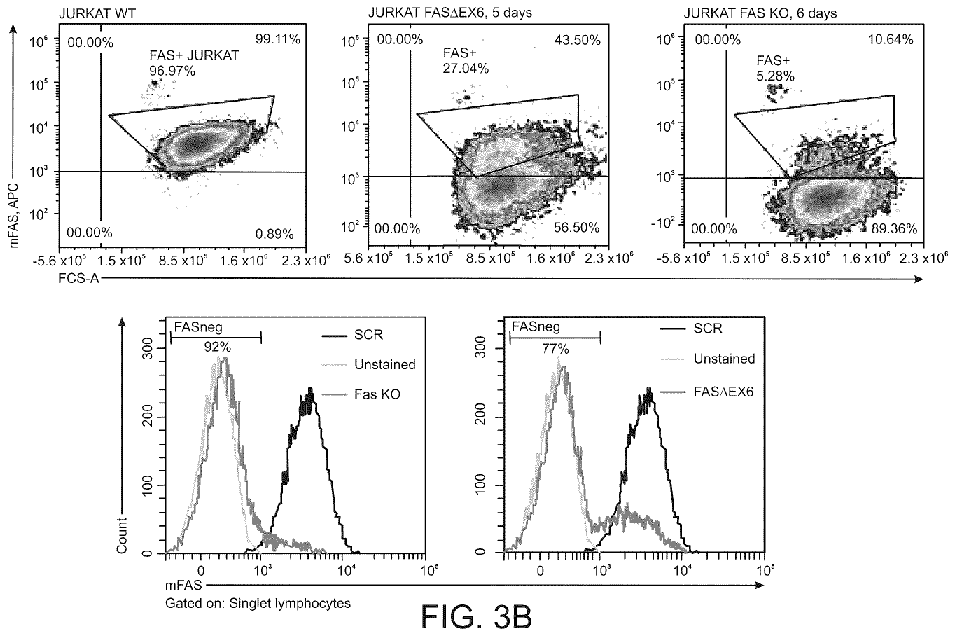

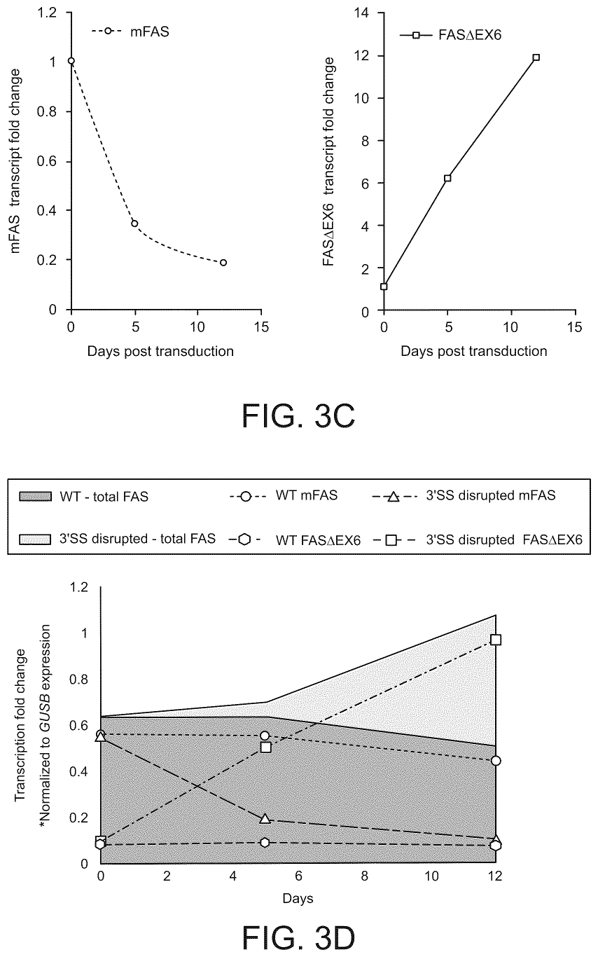

Accordingly, the inventors also used CRISPR/Cas9 system as another FAS exon 6 skipping strategy (FIG. 3A). JURKAT cells were double transduced with lentiviral particles carrying Cas9 gene and sgRNA targeting the 3′ splice site (3′SS) of FAS exon 6 (CRISPR_AS, FASΔEX6, SEQ ID NO: 15), exon 3 (CRISPR_KO, SEQ ID NO: 14), or non-targeting sgRNA (CRISPR_SCR, SEQ ID NO: 23) (Table 2). Table 2 further discloses the sgRNA full sequences as denoted by SEQ ID NOs: 16, 20, 24, the DNA long target sequences as denoted by SEQ ID NOs: 17 and 21, and the FAS DNA short target, as denoted by SEQ ID NO: 18, 22. The sgRNA plasmids contain a puromycin resistance gene or BFP as a reporter. The CRISPR_KO results in complete KO of FAS, i.e., both mFAS and sFAS transcripts. The CRISPR AS sgRNA disrupts the FAS exon 6 3′SS, leading the spliceosome to misidentify it and, therefore, to exon skipping (FASΔEX6). FAS manipulation resulted in >90% knockout six days after transduction (KO) and in 43% alternative spliced five days after transduction (FIG. 3B, upper panels). FACS analysis of single cell lymphocytes nine days after transduction, showed that about 77% of the FASΔEX6 transduced cells were negative for mFas compared to the control SCR transduced cells (FIG. 3B, lower panels). The alternative splicing was validated by qPCR, showing a significant decrease in mFAS transcript and an increase in FASΔEX6 transcript (FIG. 3C). Interestingly, the total FAS transcript in FASΔEX6 transduced cells was higher than in the control population, apparently due to ‘positive feedback loop’: the decrease in the mFAS transcript caused feedback that upregulates the FAS gene transcription. The transcription upregulation resulted in the production of a transcript that can only be spliced to the FASΔEX6 form due to the splice site disruption. Therefore, the upregulation feedback never ends, and the production of FASΔEX6 keeps increasing in a ‘positive feedback loop’. In other words, the signal for FAS transcription becomes constitutive, and there is an increase in positive feedback loop of FASΔEX6 transcript upregulation and production (FIG. 3D)).

TABLE 2
FAS exon 6 skipping - CRISPR/Cas9 3′SS disruption system in human:

| sgRNA | Guide | sgRNA full | FAS DNA long target | FAS DNA short |
|---|---|---|---|---|
| name | sequence | sequence | sequence: (target site) | target sequence |
| CRISPR_ | SEQ ID | SEQ ID | SEQ ID NO: 17 | SEQ ID NO: 18 |
| AS | NO: 15 | NO: 16 | intron5EXON6 sense | acagGATCCAGATC |
| | AAGTTA | AAGTTAGAT | tgttccaacctacagGATCCA | TAACTT |
| | GATCTG | CTGGATCCT | GATCTAACTTGGGGTG | |
| | GATCCT | GTGTTTTAG | GCTTTGTCTTCTTCTT | |
| | GT | AGCTAGAAA | TTGCCAATTCCACTA | |
| | | TAGCAAGTT | | |
| | | AAAATAAGG | | |
| | | CTAGTCCGT | | |
| | | TATCAACTT | | |
| | | GAAAAAGTG | | |
| | | GCACCGAGT | | |
| | | CGGTGC | | |
| CRISPR_ | SEQ ID | SEQ ID | SEQ ID NO: 21 | SEQ ID NO: 22 |
| KO | NO: 19 | NO: 20 | EXON3 antisense | TTCTTGGCAGGGC |
| | GACTGC | GACTGCGTG | CATGTCCTTCATCACA | ACGCAGTC |
| | GTGCCC | CCCTGCCAA | CAATCTACATCTTCTG | |
| | TGCCAA | GAAGTTTTA | CATTTGGAAGAAAAA | |
| | GAA | GAGCTAGAA | TGGGCTTTGTCTGTGT | |
| | | ATAGCAAGT | ACTCCTTCCCTTCTTG | |
| | | TAAAATAAG | GCAGGGCACGCAGTCT | |
| | | GCTAGTCCG | GGTTCATCCCCATTG | |
| | | TTATCAACT | ACTGTGCAGTCCCTA | |
| | | TGAAAAAGT | GCTTTCCTTTCAC | |
| | | GGCACCGAG | | |
| | | TCGGTGC | | |
| CRISPR_ | SEQ ID | SEQ ID | — | — |
| SCR | NO: 23 | NO: 24 | | |
| | GTATTA | GTATTACTG | | |
| | CTGATA | ATATTGGTG | | |
| | TTGGTG | GGGTTTTAG | | |
| | GG | AGCTAGAAA | | |
| | | TAGCAAGTT | | |
| | | AAAATAAGG | | |
| | | CTAGTCCGT | | |
| | | TATCAACTT | | |
| | | GAAAAAGTG | | |
| | | GCACCGAGT | | |
| | | CGGTGC | | |

### Example 4

### In Depth Characterization of Single Cell FAS Manipulated JURKAT Cells

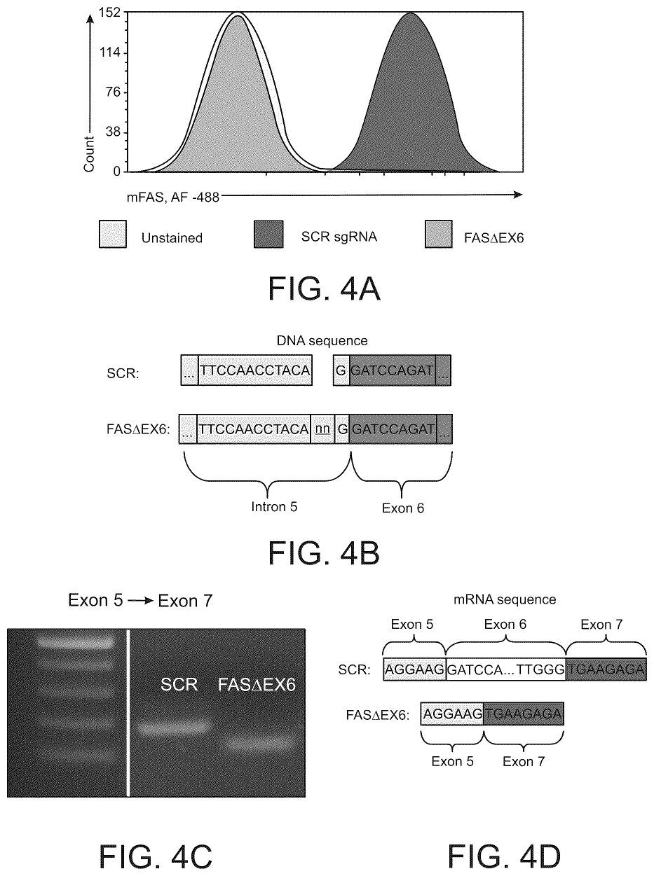

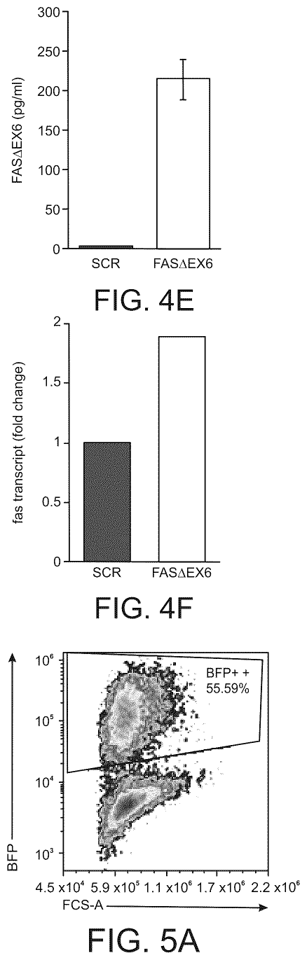

Single cell clones of JURKAT cells manipulated by FAS exon 6 skipping using CRISPR/Cas9 splice site disruption system were generated by dividing the cells to 96 wells plate in a ratio of 0.1 cells/well. Clones were analyzed for mFAS expression by FACS (FIG. 4A), and mFas negative clones were further analyzed by sequencing. The alternative splicing was first validated by DNA mutation sequencing (FIG. 4B, SEQ ID NOs: 37 and 38), FAS mRNA agarose gel electrophoresis (FIG. 4C) and mRNA sequencing (FIG. 4D, SEQ ID NOs: SEQ ID NO:40 and 41). Secretion of FASΔEX6 was also validated by ELISA (FIG. 4E).

As shown for the manipulated JURKAT cells, the positive feedback loop of FAS transcript upregulation is demonstrated also in the single cell clones (measured by qPCR as mentioned above), showing higher fas transcription (FIG. 4F).

Next, these cells are analyzed for the intracellular signaling involved in FAS AS modulation. Phosphorylated candidates for intracellular signaling are analyzed by western blotting and FACS.

### Example 5

mFAS:sFAS Ratio Alteration Through FASΔEX6 AS Enhances Survival and Functionality of PBMCs

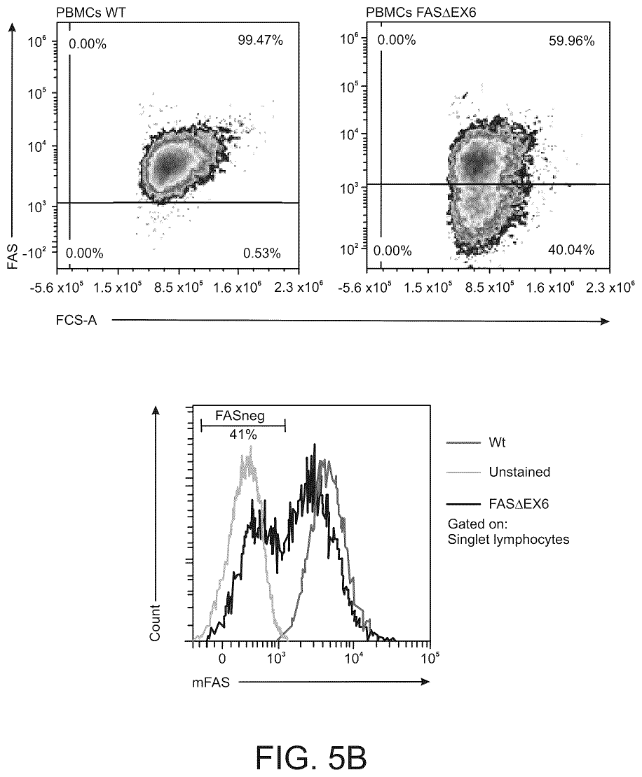

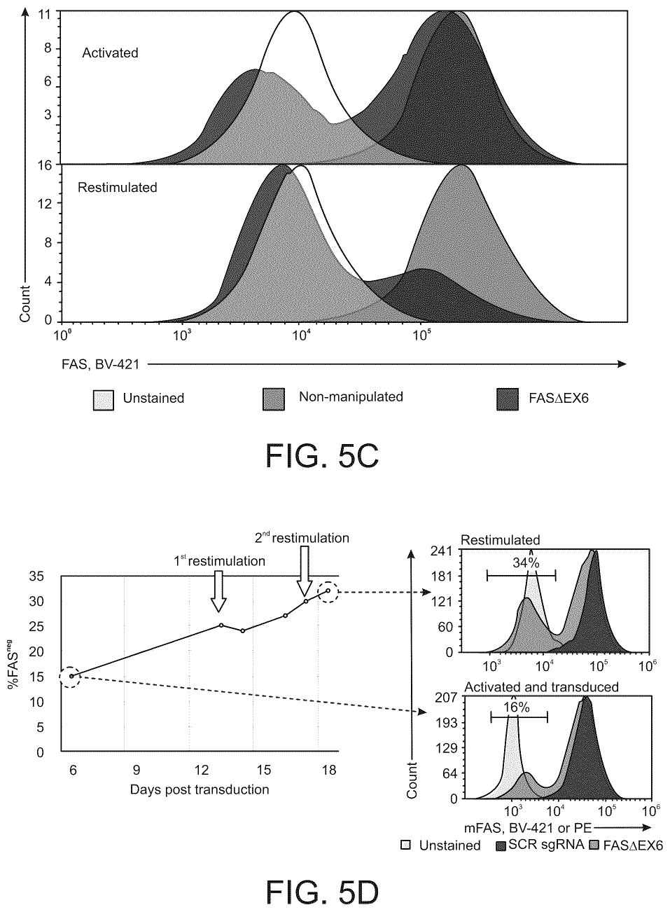

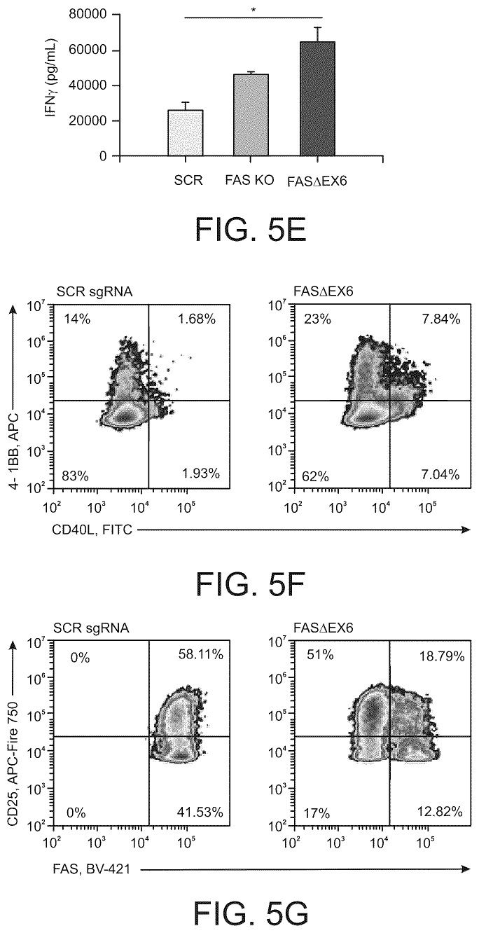

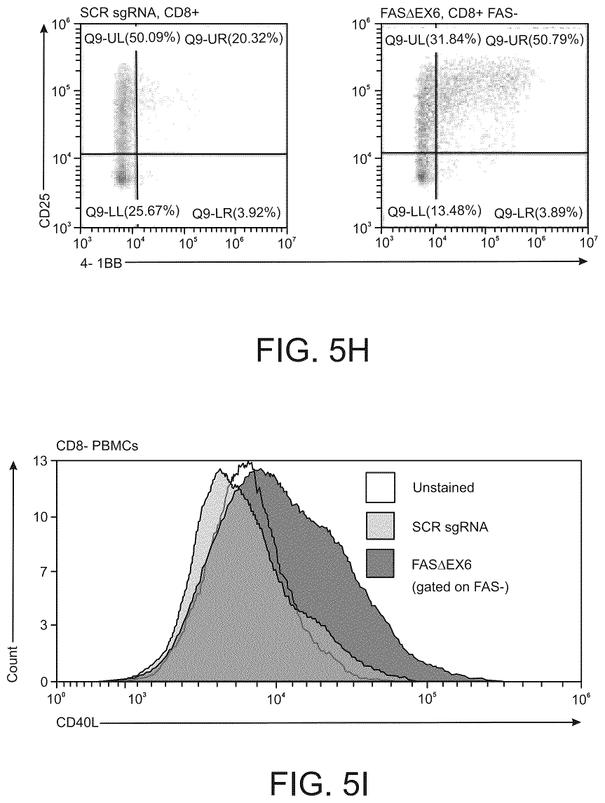

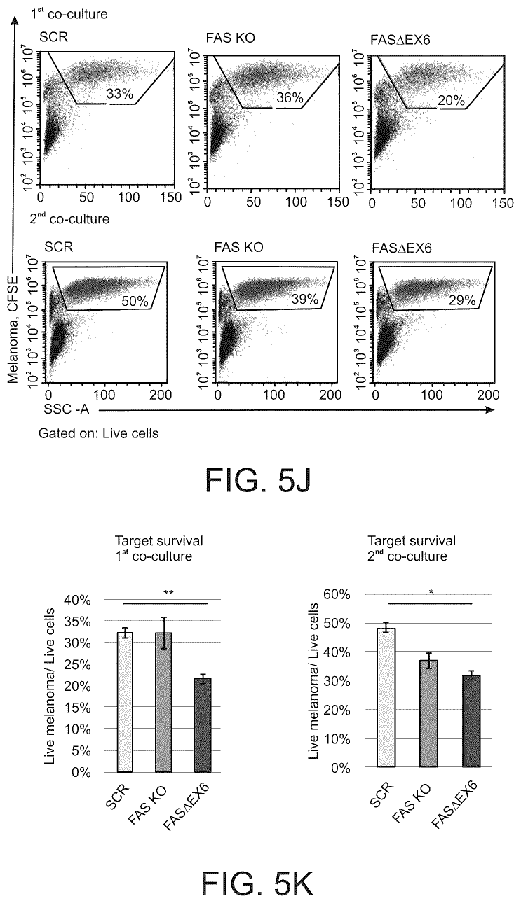

The CRISPR/Cas9 3′SS disruption system was then calibrated for activated PBMCs and reached a 56% transduction rate (FIG. 5A). Healthy donor PBMCs were transduced with CRISPR AS sgRNA (FASΔEX6), selected with puromycin, and electroporated with Cas9 protein, leading to about 40% of alternative spliced population five days after electroporation (FIG. 5B).

Following restimulation, it was expected that the percentage of the manipulated cells in the culture would remain steady. Interestingly, following two repeated restimulations (with intervals of 48 h) with 1 ug/ml plate bound anti-CD3, the alternative spliced population was positively selected (FIG. 5C, upper vs. lower panels, FASΔEX6 manipulated cells compared to non-manipulated, Cas9^{9neg} PBMCs). Similar results were observed at different FAS^{WT}:FAS^{manipulated} ratios, i.e., initially 16% of the FASΔEX6 manipulated cells were FAS negative, and this subpopulation increased to 34% following two restimulations (FIG. 5D). Moreover, FASΔEX6 T cells (PBMCs from FIG. 5B) secreted more IFNγ following restimulation (for 48 h) compared to the FAS KO cells (FIG. 5E).

Furthermore, he alternative spliced population has higher expression of the activation markers CD25 (FIG. 5G), 4-1BB and CD40L (FIG. 5F). Similarly, both CD8+ and CD8− subpopulations of FASΔEX6 transduced cells also showed higher expression of the activation markers CD25 and 4-1BB (FIG. 5H), and CD40L (FIG. 5I), compared to CD8+ and CD8− subpopulations in the control cells (SCR sgRNA).

To further examine FASΔEX6 function, CRISPR/Cas9 FAS 3SS disrupted (25% FAS^{neg}), FAS KO (25% FAS^{neg}) and SCR healthy donor PBMCs were transduced with retrovirus encoding NY-ESO1 TCR, that recognizes the NY-ESO antigen (FASΔEX6NY-ESO-TCR+/FAS KO-ESO-TCR/+SCR ESO-TCR+PBMCs). The transduced PBMCs were then cocultured twice in 1:1 ratio with the target cell line 624mel (HLA-A2+/MART-1+gp100+) presenting the NY-ESO1 melanoma antigen. Co-culturing of the melanoma target cells with FASΔEX6 cells resulted in 20% of live melanoma cells following 24 hours of incubation relative to 33%, 36% live melanoma cells when co-cultured with SCR, FAS KO cells (FIG. 5J upper panel—representative scattering plot and FIG. 5K left panel). Second co-culturing of the melanoma target cells with FASΔEX6 cells resulted in 29% of live melanoma cells relative to 50%, 39% live melanoma cells when co-cultured with SCR, FAS KO cells (FIG. 5J lower panel—representative scattering plot and FIG. 5K right panel).

### Example 6

Advantage of Manipulated FASΔEX6 PBMCs in Pre-ACT Context that Includes Rapid Expansion

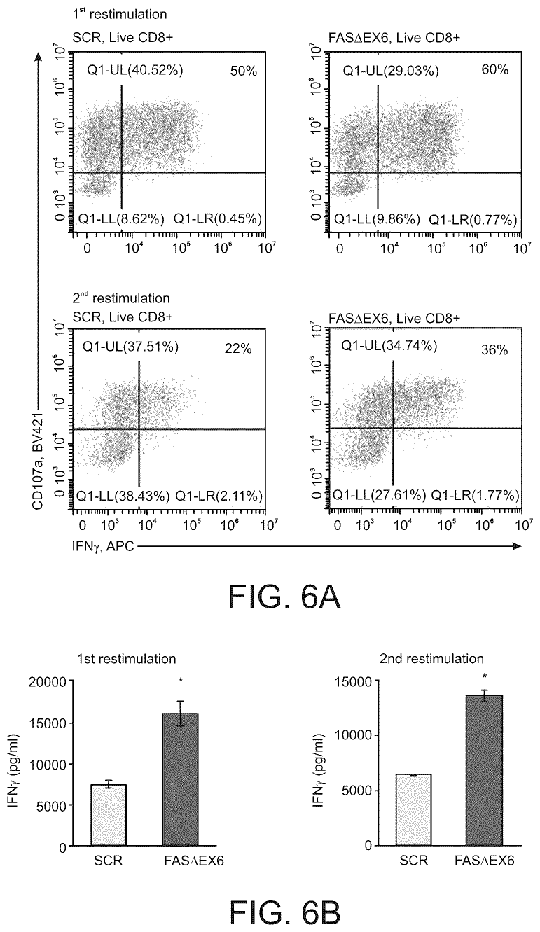

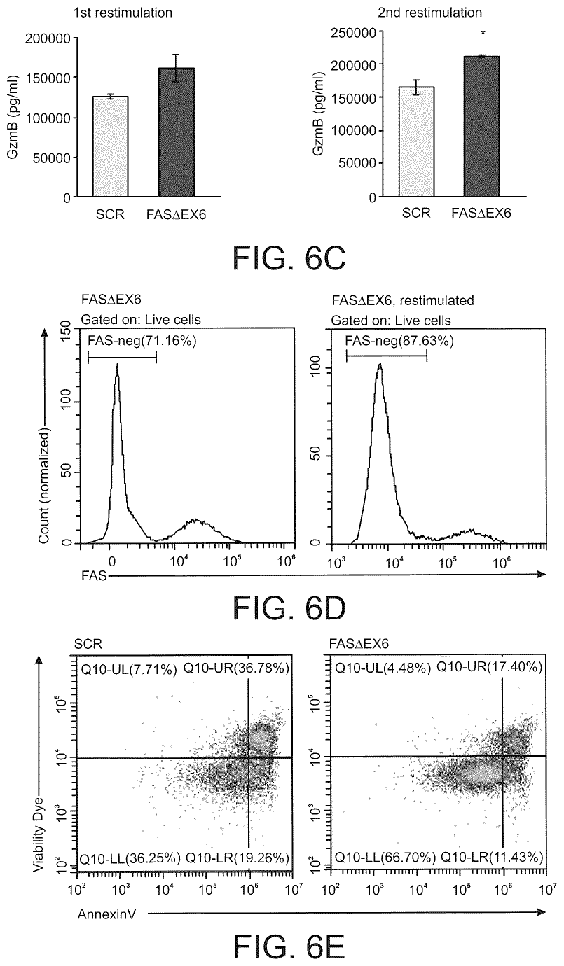

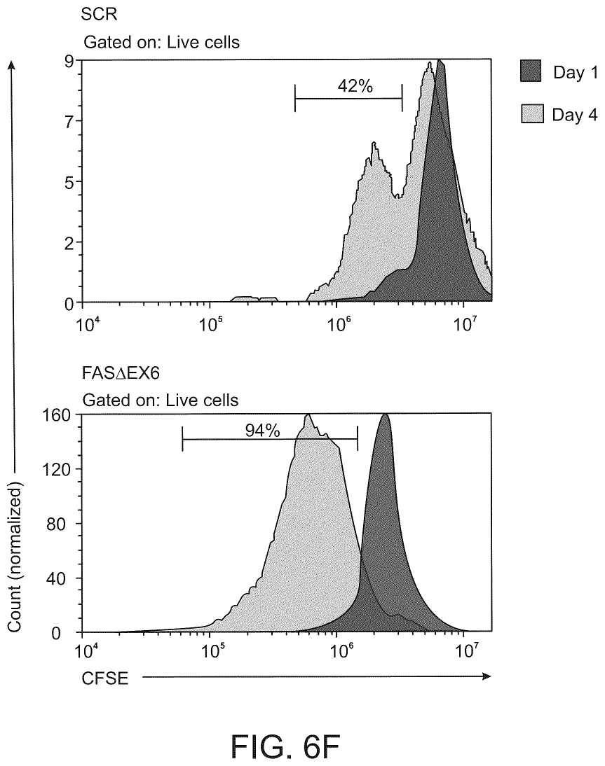

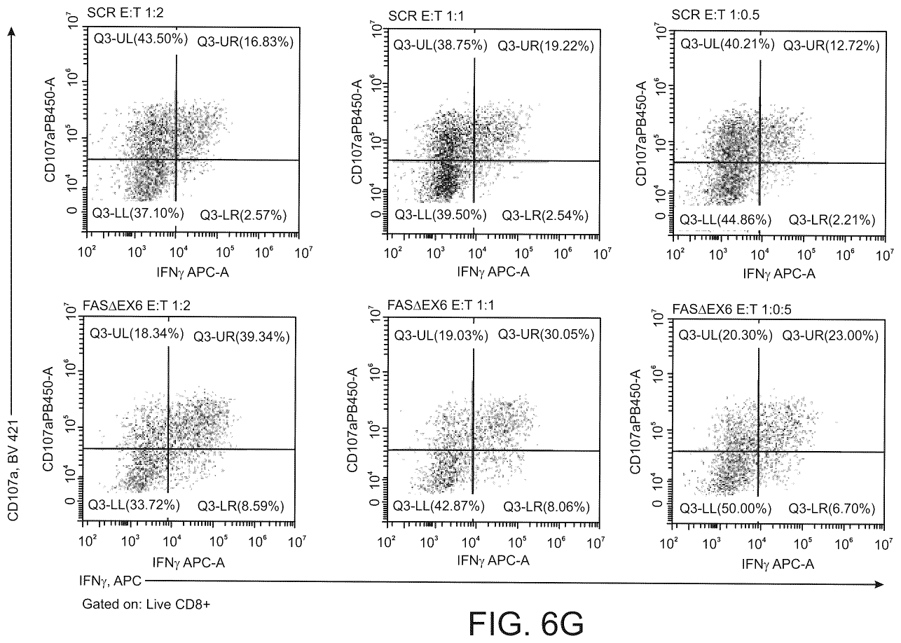

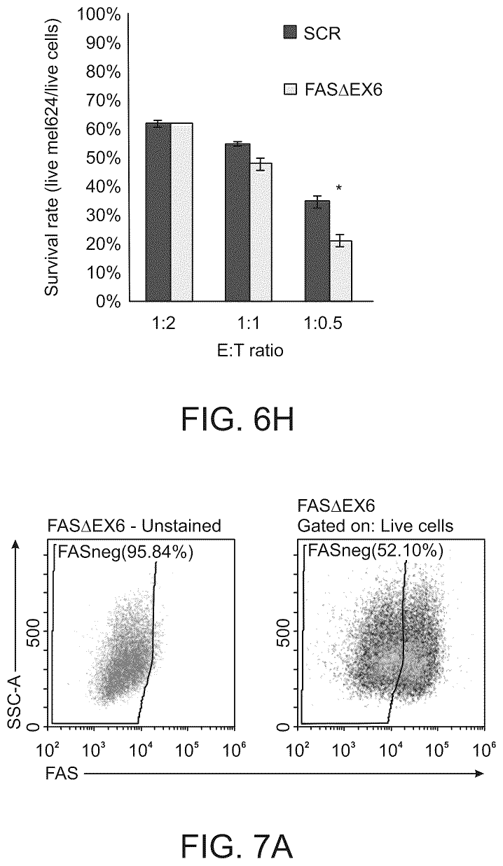

FASΔEX6NY-ESO-TCR+PBMCs were expanded by activation with 30 ng/mL of anti-CD3 and screened PBMCs as feeders for 12 days [Dudley, Mark E. et al., Journal of immunotherapy 26 (4), 332-342 (2003); Eisenberg, G., et al., J Immunol 190 (11): 5856-5865 (2013)]. Fresh medium containing IL2 was added on day 5 and every other day thereafter. Expanded FASΔEX6NY-ESO-TCR+ were activated by anti-CD3 and anti-CD28 antibodies as detailed for the FAS^{manipulated} PBMCs. The functional and survival advantages of the FASΔEX6 population were similar to those shown in the other systems. For example, following two consecutive restimulations, the percentage of CD8+ subpopulation of FASΔEX6NY-ESO-TCR+ cells which co-express CD107a and IFNγ, was higher compared to the percentage of CD8+ subpopulation of SCR-ESO-TCR+ cells indicating the increased ability to degranulate and produce cytokines. In the first restimulation, 60% of CD8+ subpopulation in the manipulated FASΔEX6 were double positive compared to 50% of CD8+ subpopulation in the control SCR population, and in the second restimulation, 36% of CD8+ subpopulation in the manipulated FASΔEX6 compared to 22% in the control SCR population, respectively (FIG. 6A). Similarly, secretion of IFNγ (FIG. 6B), and GranzymeB (FIG. 6C), was more pronounced in FASΔEX6 cells compared to the control cells, indicating increased cytotoxic capabilities. In addition, as shown for the healthy donor PBMCs transduced with FASΔEX6 (FIG. 5C), the FASΔEX6NY-ESO-TCR+ cells showed an advantage in cell survival: following rapid expansion, the FAS negative subpopulation constituted 71% of the total population, and following two restimulations, the FAS negative subpopulation composed of about 88% of the total population, suggesting a positive selection process (FIG. 6D). In another experiment, cells were stained with CFSE and following restimulation with antibodies, stained for AnnexinV and viability dye. FASΔEX6NY-ESO-TCR+ cells, showed less apoptosis compared to the control cells with 67% of the FASΔEX6NY-ESO-TCR+ cells showing full viability (double negative staining), compared to 36% in the SCR population (FIG. 6E). The cells were further analyzed 4 days later and showed increased proliferation capacity compared to SCR cells, with 94% dividing cells, compared to 42% in the SCR population (FIG. 6F).

To examine the functional capabilities of the transduced cells against target cells in repeated restimulations context, the cells were pre-activated with 1 ug/ml anti-CD3 and anti-CD28 antibodies for 24 h, rested for 24 h and co-cultured with 624mel target cells at a different E:T ratios. FASΔEX6 CD8+ T cells presented functional advantages in all E:T ratios over the control cells: 39%, 30% and 23% of the FASΔEX6 CD8+ T cells co-express CD107a and IFNγ compared to only 17%, 19% and 13% in the SCR population, for 1:2, 1:1 and 1:0.5 E:T ratios, respectively (FIG. 6G). These results were accompanied by the reduced survival rate of the target mel624 cells when co-cultured with FASΔEX6 T cells, compared to the survival rate of the target mel624 cells co-cultured with SCR control cells, indicating improved cytotoxicity and/or survivability of FASΔEX6 Pmel-1 cells (FIG. 6H).

### Example 7

### In Vivo Mouse Model for Evaluation of FASΔEX6 Adoptive Cell Transfer—(ACT).

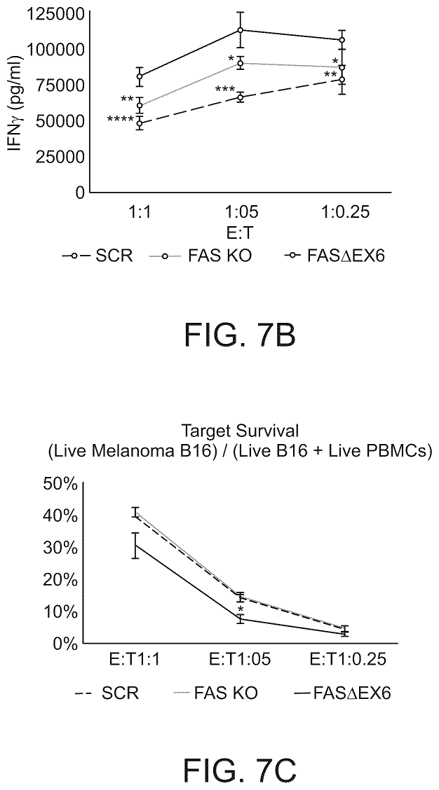

To evaluate the therapeutic effect of the disclosed cells and methods, mice that carry a rearranged T-cell receptor (TCR) Vbeta13, specific for the 9-mer epitope 25-32 from murine Pmel 17, the homolog of human gp100 were used. Specifically, CD8+Pmel-Cas9 mice splenocytes were isolated, activated with anti-CD3 and anti-CD28 antibodies and transduced with retro- and/or lenti-virus coding for gRNA that targets FAS exon 6 splice site (mCRISPR_AS, FASΔEX6, SEQ ID NO:42), resulting in FASΔEX6 Pmel-1 splenocytes (FIG. 7A). gRNA targeting exon 2 is used for FAS-KO (mCRISPR_KO, SEQ ID NO:46) and the non-targeting sgRNA (mCRISPR_SCR, SEQ ID NO: 50) served as control (Table 3).

Table 3 further discloses the mouse sgRNA full sequences as denoted by SEQ ID NOs: 43, 47, 51, the mouse FAS DNA long target sequences as denoted by SEQ ID NOs: 44 and 48, and the mouse FAS DNA short target, as denoted by SEQ ID NO: 45 and 49. The sgRNA plasmids contain a puromycin resistance gene or BFP as a reporter. The mCRISPR_KO results in complete KO of FAS, i.e., both mFAS and sFAS transcripts. The mCRISPR_AS sgRNA disrupts the FAS exon 6 3′SS, leading the spliceosome to misidentify it and, therefore, to exon skipping (FASΔEX6). Cells were incubated with IL2 (100 IU/mL) and fresh medium containing IL2 was added every other day. Mouse melanoma B16-F10/mhgp100 were used as target cells (B16-F10 melanoma cells transduced with pMSGV1 retrovirus encoding a chimeric mouse gp100 with the human gp10025-33 sequence [Hanada K I, Yu Z, Chappell G R, Park A S, Restifo N P. An effective mouse model for adoptive cancer immunotherapy targeting neoantigens. JCI Insight. 2019 May 16; 4(10):e124405. doi: 10.1172/jci.insight.124405. PMID: 31092734; PMCID: PMC6542630]. The cells were cultured in RPMI1640 supplemented with 10% heat-inactivated FCS, 2 mmol/L 1-glutamine, and combined antibiotics (all from Invitrogen Life Technologies).

The inventors performed an ex-vivo assay to test FASΔEX6 Pmel-1 function. FASΔEX6 Pmel-1 were co-cultured with B16-F10/mhgp100 target cells in different E:T ratios for 24 h. Indeed, FASΔEX6 Pmel-1 co-cultured with the target cells showed increased IFNγ secretion in all E:T ratios, compared to that observed in wither the FAS-KO or SCR (mCRISPR_SCR) Pmel-Cas9 T cells (FIG. 7B). Interestingly, in all E:T ratios, the survival rate of the target B16-F10/mhgp100 cells was lower when co-cultured with FASΔEX6 Pmel-1 cells (FIG. 7C) compared to co-cultured with either FAS KO cells or the SCR control cells, indicating better cytotoxicity and/or survivability for FASΔEX6 Pmel-1, as shown for human FASΔEX6 T cells.

Adoptive cell transfer experiments are next performed. More specifically, B16-F10 and/or B16-F10/mhgp100 mouse melanoma cells (0.4×10⁶) are injected subcutaneously into the back of C57BL 6 mice on day −7. WT, FAS-KO or FASΔEX6 CD8+Pmel-1 mouse splenocytes are incubated in the presence of IL2 100 IU/mL. Fresh medium containing IL2 is added every other day. On day −1, cells are sorted for BFP+. The following day, 2-10×10⁶ cells are adoptively transferred intravenously to the tail vein of 500 CGy-irradiated and/or chemotherapy-treated tumor-bearing mice. The following days, IL2 is intraperitoneally administered, and the mice are monitored by measuring tumor size in two perpendicular diameters three times per week. Mice are sacrificed at the end of the experiment and tumors and spleens are harvested for further analysis by flow cytometry.

TABLE 3
FAS exon 6 skipping - CRISPR/Cas9 3′SS disruption system in mouse

| sgRNA | Guide | sgRNA full | FAS DNA long target | FAS DNA short |
|---|---|---|---|---|
| name | sequence | sequence | sequence: (target site) | target sequence |
| mCRISPR_ | SEQ ID | SEQ ID | SEQ ID NO: 44 | SEQ ID NO: 45 |
| AS | NO:42 | NO: 43 | EXON6intron5 antisense | gcgaatattggatattttgt |
| | ACAAAAT | ACAAAATAT | CCATAGGCGATTTCTGG | |
| | ATCCAAT | CCAATATTC | GACctgcgaatattggatattttgtat | |
| | ATTCGC | GCGTTTTAGA | | |
| | | GCTAGAAATA | | |
| | | GCAAGTTAAA | | |
| | | ATAAGGCTAG | | |
| | | TCCGTTATCA | | |
| | | ACTTGAAAAA | | |
| | | GTGGCACCGA | | |
| | | GTCGGTGC | | |
| mCRISPR_ | SEQ ID | SEQ ID | SEQ ID NO: 48 | SEQ ID NO: 49 |
| KO | NO: 46 | NO: 47 | EXON2 antisense | TGAGTATGAACTC |
| | CAGTTAA | CAGTTAAGA | GCTATTAGTACCTTGAG | TTAACTG |
| | GAGTTCA | GTTCATACTC | TATGAACTCTTAACTGT | |
| | TACTCA | AGTTTTAGAG | GAGCCAGCAAGCAC | |
| | | CTAGAAATAG | | |
| | | CAAGTTAAAA | | |
| | | TAAGGCTAGT | | |
| | | CCGTTATCAA | | |
| | | CTTGAAAAAG | | |
| | | TGGCACCGAG | | |
| | | TCGGTGC | | |
| mCRISPR_ | SEQ ID | SEQ ID NO: | — | — |
| SCR | NO: 50 | 51 | | |
| | ATATCCA | ATATCCACCT | | |
| | CCTAATC | AATCGAATA | | |
| | GAATAA | AGTTTTAGAG | | |
| | | CTAGAAATAG | | |
| | | CAAGTTAAAA | | |
| | | TAAGGCTAGT | | |
| | | CCGTTATCAA | | |
| | | CTTGAAAAAG | | |
| | | TGGCACCGAG | | |
| | | TCGGTGC | | |

### Example 8

### In Vivo NOD Scid Gamma (NSG) Mouse Model for Evaluation of FASΔEX6 Human Adoptive Cell Transfer (ACT)

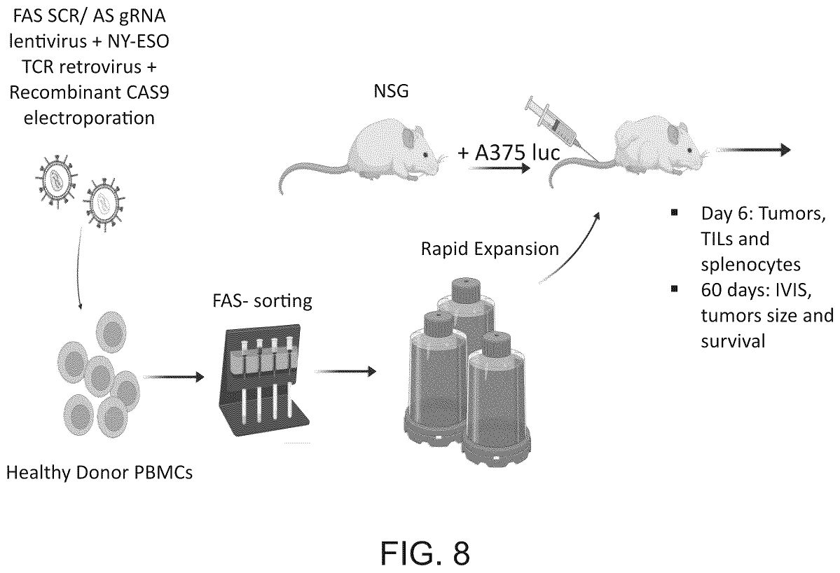

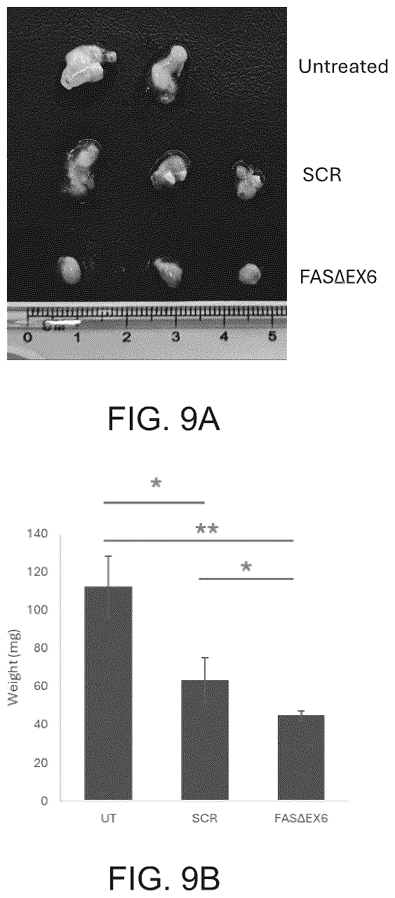

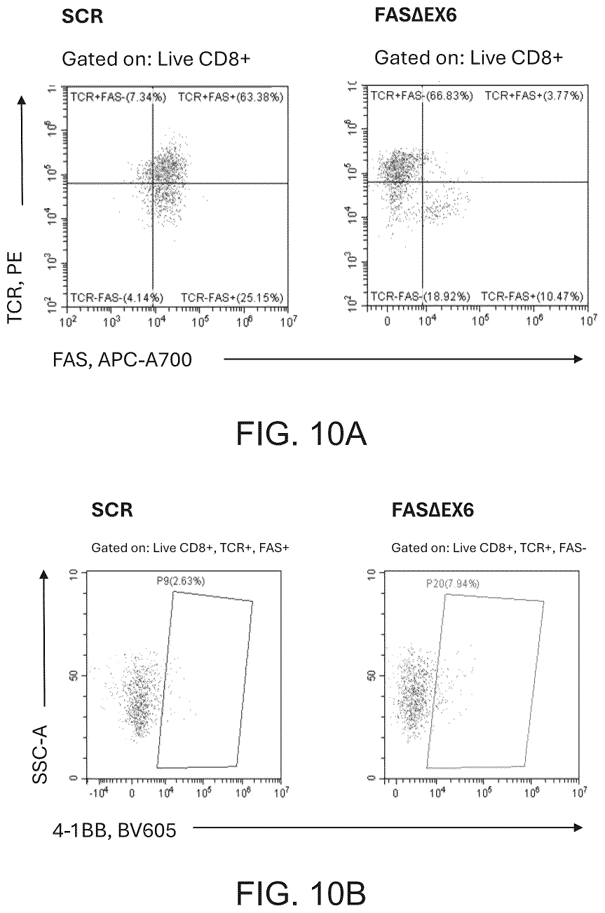

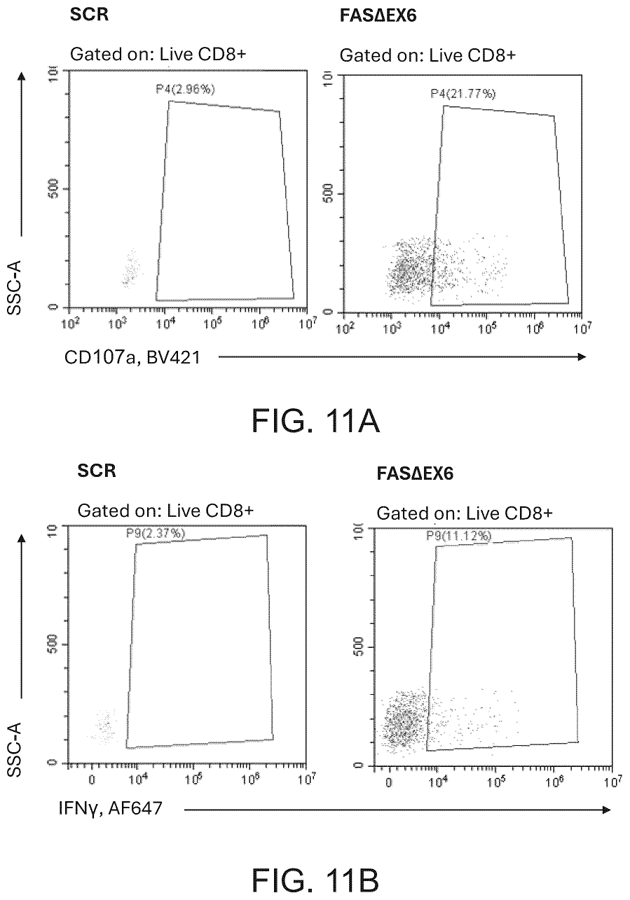

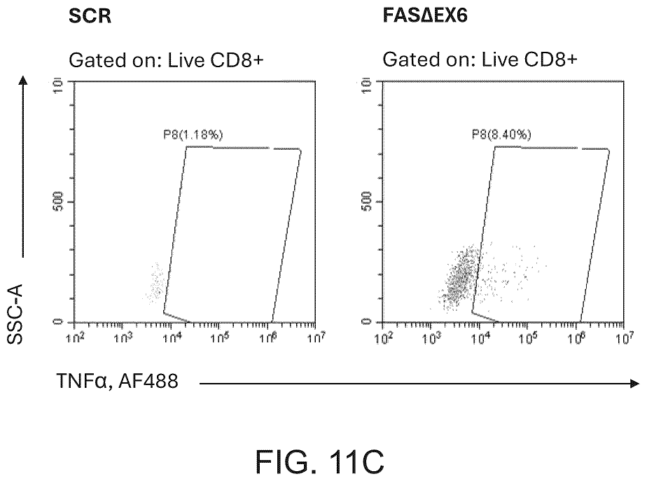

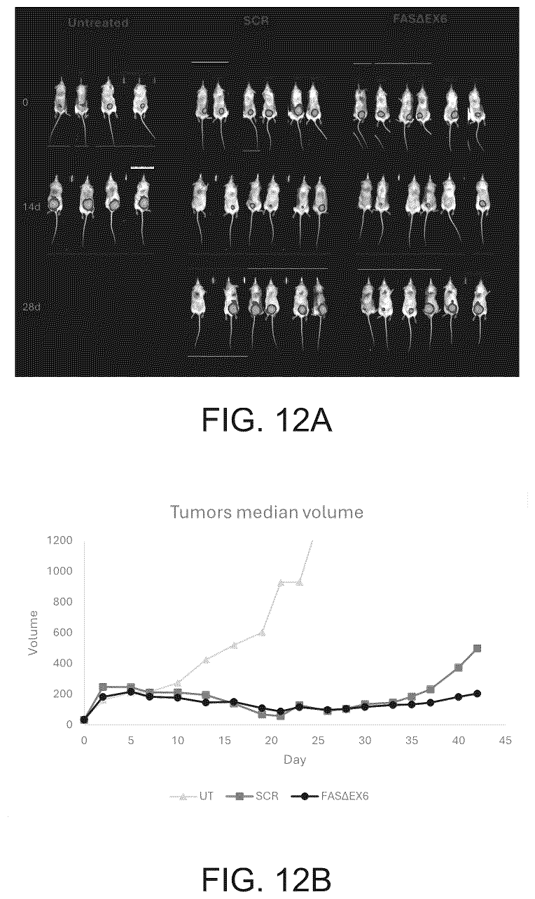

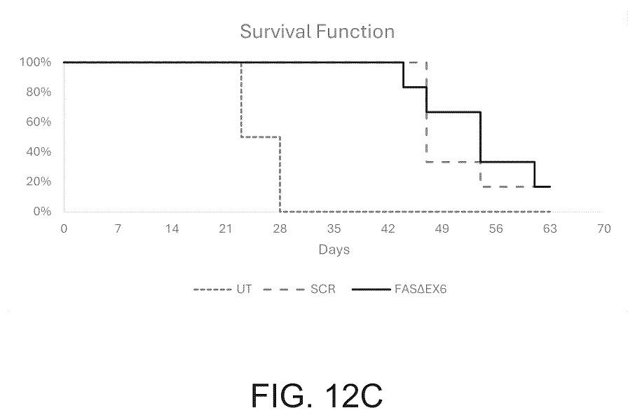

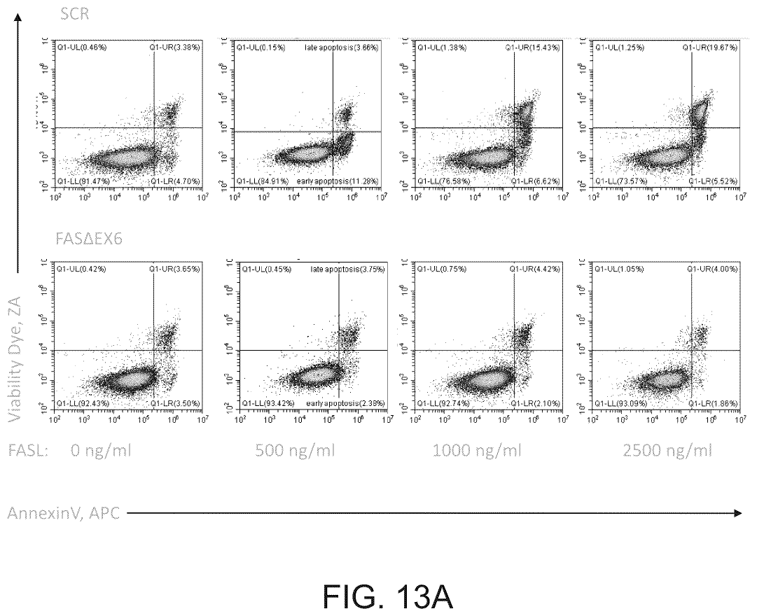

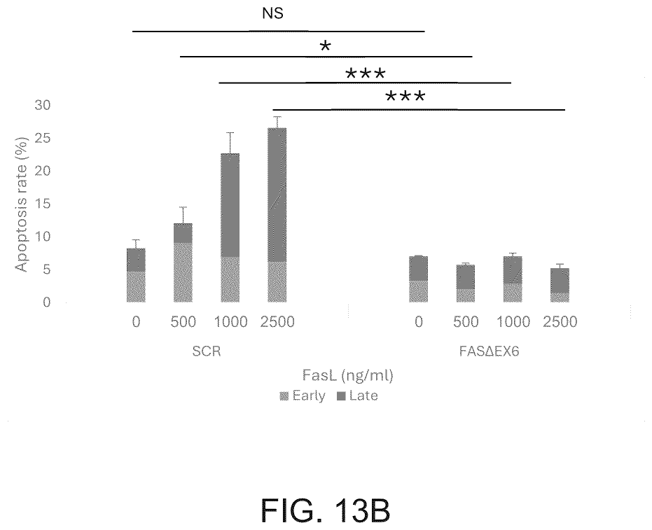

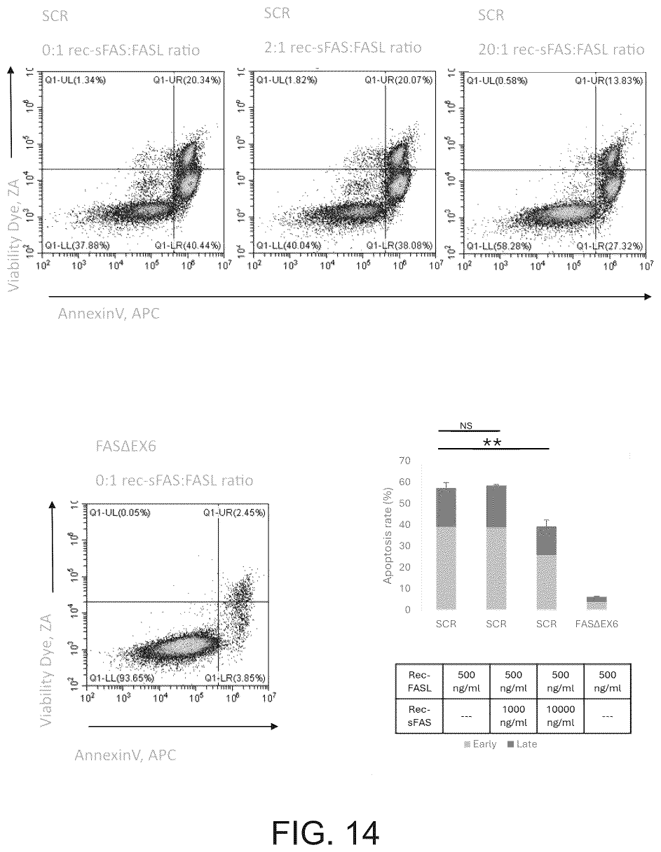

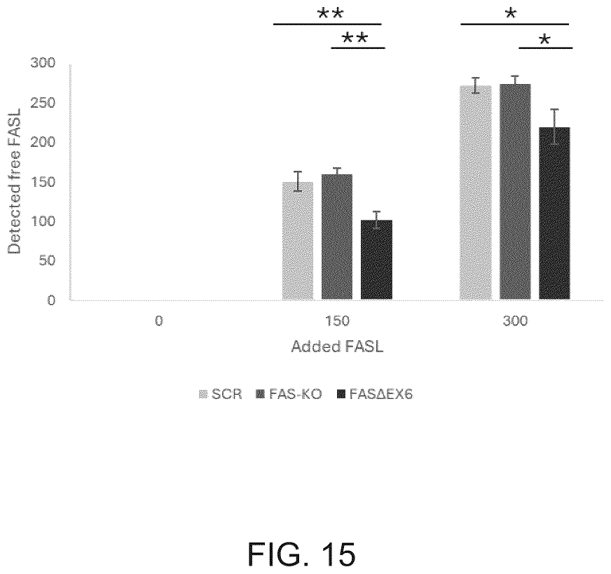

To evaluate the therapeutic effect of the human FASΔEX6 T cells and their relevance to ACT-based immunotherapy, an NSG xenograft model was established using luciferase-expressing human A375 melanoma cells, as illustrated in FIG. 8. Briefly, healthy donor PBMCs were transduced with retroviral vector encoding the NY-ESO-1 TCR and lentiviral constructs encoding either a non-targeting sgRNA (SCR, SEQ ID NO: 23, or an sgRNA targeting the 3′ splice site of exon 6 (FASΔEX6, CRISPR_AS, SEQ ID NO:15). Electroporation with recombinant Cas9 protein was performed, and cells were expanded using a rapid expansion protocol. On day −8, 1×10⁶ A375-luc melanoma cells were injected subcutaneously into the back of NSG mice. On day 0, 10×10⁶ manipulated human T cells were intravenously administered via tail vein injection. Subsequently, IL2 was intraperitoneally administered for 5 days (500,00 IU per day), and tumor progression was monitored by caliper measurements three times weekly and bioluminescence imaging (IVIS) every two weeks. Bioluminsecence imaging showed differences between FASscr and FASΔEX6 groups with 3 out of 6 animals in the FASΔEX6 group displaying reduced tumor growth compared to only one mouse in the SCR group at day 28 post ACT. Tumor growth was also followed by caliper measurements depicting delayed tumor growth in the FASΔEX6 group.

Mice were euthanized at day 6 post-transfer for analysis. As shown in FIGS. 9A-9B, at day 6, tumor weight was significantly lower in FASΔEX6-treated mice compared to untreated or the SCR treated groups. Reduction in the tumor size in the SCR group reflects the action of the T cells that express the NY-ESO-1 antigen expressed by the tumor cells. As shown in FIG. 10A, TILs from tumors treated with FAS-manipulated T cells demonstrated significantly reduced FAS expression compared to SCR controls (TCR+FAS⁺ populations: 63%, 5%, and 4% for SCR, FAS-KO, and FASΔEX6 T cell recipients, respectively). Interestingly, increased expression of the activation marker 4-1BB was observed in FASΔEX6 TILs compared to SCR or FAS-KO controls (FIG. 10B), suggesting enhanced in vivo T cell activation.

T lymphocytes isolated from splenic tissue were restimulated ex vivo with anti-CD3 and anti-CD28 antibodies for 6 hours. FASΔEX6 CD8+ T cells demonstrated higher expression of CD107a (21.77%, FIG. 11A), IFNγ (11.12%, FIG. 11B), and TNFα (8.4%, FIG. 11C) compared to SCR control T cells (˜3%, 2%, and 1%, respectively) (FIGS. 11A-11C), consistent with enhanced effector function.

Longitudinal analysis of tumor burden revealed that mice treated with FASΔEX6 T cells displayed delayed tumor growth as shown in both bioluminescence imaging (FIG. 12A) and by caliper-based tumor volume measurements (FIG. 12B). While SCR-treated mice also demonstrated reduced tumor volume and improved survival relative to untreated group, consistent with the fact that these cells express a TCR specific for a melanoma-associated antigen (NY-ESO-1) the FASΔEX6 group nonetheless exhibited a clear advantage, with prolonged survival compared to either SCR-treated or untreated groups (FIG. 12C).

These results provide in vivo validation that FASΔEX6-modified human T cells exhibit improved activation, cytokine secretion, cytotoxic function, and therapeutic efficacy in ACT, in line with the ex vivo and murine data previously presented.

### Example 9

Resistance of FASΔEX6-Modified T Cells to FASL-Induced Apoptosis and Contribution of Secreted sFAS to Ligand Sequestration

To further investigate the functional consequences of exon 6 skipping in FASΔEX6 cells, single-clone SCR and FASΔEX6 JURKAT cells were analyzed for their sensitivity to FASL-induced apoptosis. Cells were incubated for 24 hours with increasing concentrations of recombinant FAS ligand (0, 500, 1000, or 2500 ng/mL) and subsequently stained with Annexin V (early apoptosis) and a viability dye (late apoptosis) to assess apoptosis by flow cytometry. SCR cells demonstrated a dose-dependent increase in apoptosis, reaching 25% apoptotic cells at the highest FASL concentration (as shown in FIG. 13A, upper panel and FIG. 13B). In contrast, FASΔEX6 cells maintained a constant low apoptosis rate (˜6%) across all FASL concentrations, indicating enhanced resistance to FAS-mediated cell death (as shown in FIG. 13A, lower panel and FIG. 13B).

To examine the role of secreted sFAS in modulating and reducing apoptosis, SCR and FASΔEX6 JURKAT clones were exposed to recombinant FASL (500 ng/mL) in the presence or absence of increasing concentrations of recombinant soluble FAS (sFAS) at sFAS:FASL ratios of 0:1, 2:1, and 20:1. SCR cells showed partial protection from apoptosis only at the highest

sFAS dose, with apoptosis decreasing from 60% to 41% (FIG. 14, sum of early and late apoptosis). In contrast, FASΔEX6 cells remained highly resistant to apoptosis (6%) even without exogenous sFAS, supporting the superior protective effect of the endogenous sFAS variant (FIG. 14, sum of early and late apoptosis).

To confirm secretion of functional sFAS by FASΔEX6 cells, JURKAT clones of SCR, FAS-KO, and FASΔEX6 were activated with 200 ng/mL PMA and 300 ng/mL ionomycin for 24 hours. The supernatant was collected and incubated with recombinant FASL. Levels of unbound (free) FASL were then measured by ELISA competition assay. FASΔEX6 supernatants showed reduced levels of free FASL compared to SCR and FAS-KO controls, indicating effective binding and sequestration of FASL by secreted sFAS from FASΔEX6 cells (FIG. 15).

These findings support the conclusion that FASΔEX6-modified T cells are more resistant to FASL-induced apoptosis both by reducing membrane-bound FAS expression and through secretion of functional sFAS capable of ligand neutralization.

1. A non-naïve cell of the T lineage manipulated for enhanced skipping of exon 6 of the Fas (tumor necrosis factor (TNF) receptor superfamily, member 6) gene, or a cell population comprising at least one of said cell, wherein said non-naïve manipulated cell of the T lineage predominantly expresses a soluble form of Fas (sFas).

2. The non-naïve cell of the T lineage according to claim 1, wherein said cell is manipulated by at least one splicing modulating agent or any vector, vehicle, matrix, nano- or micro-particle or composition comprising said at least one agent, said splicing modulating agent comprising at least one nucleic acid molecule that targets at least one target nucleic acid sequence participating directly or indirectly in at least one splicing event of the Fas gene, optionally, at least one of:
(a) wherein said at least one target nucleic acid sequence that participates directly or indirectly in at least one splicing event comprises at least one of: a splicing junction, a splice donor site, a splice acceptor site, an exonic splicing enhancer and an exonic splicing silencer, an intronic splicing enhancer, an intronic splicing silencer, and branch site of said FAS target gene; and
(b) wherein said target nucleic acid sequence is comprised within and/or comprises at least one of: 5′ splice site (5′SS) of exon 6, 3′ splice site (3′SS) of exon 6, exon 6, at least one intron located upstream and/or downstream to said exon 6, and/or at least one splicing junction flanking said exon 6 of the FAS gene.

3. (canceled)

4. (canceled)

5. The non-naïve cell of the T lineage according to claim 2, wherein said splicing modulating agent comprises at least one of:
(a) at least one oligonucleotide comprising a nucleic acid sequence complementary to at least part of said target nucleic acid sequence; and
(b) at least one nucleic acid sequence comprising at least one guide RNA (gRNA) that targets at least one protospacer within said target nucleic acid sequence, or any nucleic acid sequence encoding said gRNA, said gRNA guiding at least one programmable engineered nuclease (PEN) to said target nucleic acid sequence.

6. The non-naïve cell of the T lineage according to claim 5, wherein at least one of:
(a) said splicing modulating agent comprises at least one oligonucleotide, and wherein said oligonucleotide is an antisense oligonucleotide (ASO) and/or a splice switching antisense oligonucleotide (SSO), comprising at least fifteen contiguous nucleobases complementary to at least part of said at least one target nucleic acid sequence that participates directly or indirectly in at least one splicing event,
(b) wherein said SSO and/or ASO comprise nucleic acid sequence complementary to the nucleic acid sequence of intron 5/exon 6 and/or to exon 6/intron 6 of the Fas gene; and
(c) wherein said ASO and/or SSO are chemically modified.

7. (canceled)

8. (canceled)

9. The non-naïve cell of the T lineage according to claim 5, wherein said PEN comprises at least one clustered regulatory interspaced short palindromic repeat (CRISPR)/CRISPR associated (cas) protein, and wherein said splicing modulating agent comprises:
(a) at least one nucleic acid sequence comprising at least one gRNA, or any nucleic acid sequence encoding said gRNA; or any kit, composition, vector or vehicle comprising said gRNA or nucleic acid sequence encoding said gRNA; and optionally
(b) at least one CRISPR/cas protein, or any nucleic acid molecule encoding said Cas protein, or any kit, composition, vector or vehicle comprising said CRISPR/cas protein or nucleic acid sequence encoding said CRISPR/cas protein: optionally, wherein said gRNA targets at least one protospacer within at least one of: the 5′ splice site (5′SS) of exon 6 and/or the 3′ splice site (3′SS) of exon 6 of the Fas gene.

10. (canceled)

11. The non-naïve cell of the T lineage according to claim 1, wherein at least one of:
(a) said cell expresses reduced levels of membrane Fas (mFas),
(b) said cell is further engineered to express at least one receptor molecule, said at least one receptor molecule comprising at least one target binding domain specific against at least one target antigen; and
(c) said at least one cell is characterized by at least one of: (i) reduced expression of mFas: (ii), increased cytokine secretion: (iii) increased expression of activation markers: (iv) increased cell survival and/or cell viability; (v) increased cytotoxicity; and/or (vi) reduced expression of exhaustion markers.

12. (canceled)

13. (canceled)

14. A composition comprising at least one non-naïve cell of the T lineage as define in claim 1, wherein said cell is manipulated for enhanced skipping of exon 6 of the Fas gene, or a cell population comprising at least one of said cell, wherein said non-naïve manipulated cell of the T lineage predominantly expresses sFas;
said composition optionally further comprising at least one of pharmaceutically acceptable carrier/s, diluent/s, excipient/s and additive/s.

15. (canceled)

16. A method for treating, ameliorating, inhibiting or delaying the onset of a pathologic disorder in a mammalian subject, said method comprising the step of administering to said subject an effective amount of at least one of:
(a) at least one non-naïve cell of the T lineage manipulated for enhanced skipping of exon 6 of the Fas gene, or a cell population comprising at least one of said cell, wherein said non-naïve manipulated cell of the T lineage predominantly expresses sFas;
(b) at least one splicing modulating agent or any vector, vehicle, matrix, nano- or micro-particle or composition comprising said at least one agent, said splicing modulating agent comprising at least one nucleic acid molecule that targets at least one target nucleic acid sequence participating directly or indirectly in at least one splicing event of the Fas gene; and/or
(c) a composition comprising said non-naïve cell/s of the T lineage of (a) and/or the at least one splicing modulating agent of (b).

17. The method according to claim 16, wherein said pathologic (a) disorder is at least one of: a proliferative disorder, an inflammatory disorder, an infectious disease caused by a pathogen, an autoimmune-disease, a cardiovascular disease and/or a neurodegenerative disorder, optionally, said proliferative disorder is at least one malignant neoplastic disorder, optionally said neoplastic disorder is at least one hematological malignancy, and/or at least one solid tumor.

18. (canceled)

19. The method according to claim 16, wherein at least one of:
(a) said at least one target nucleic acid sequence that participates directly or indirectly in at least one splicing event comprises at least one of: a splicing junction, a splice donor site, a splice acceptor site, an exonic splicing enhancer, splicing silencer, an intronic splicing enhancer and an intronic splicing silencer of said FAS target gene; and
(b) said target nucleic acid sequence is comprised within and/or comprises at least one of: 5′SS of exon 6, 3′SS of exon 6, exon 6, at least one intron located upstream and/or downstream to said exon 6, and/or at least one splicing junction flanking said exon 6.

20. (canceled)

21. The method according to claim 16, wherein said splicing modulating agent comprises at least one of:
(a) at least one oligonucleotide comprising a nucleic acid sequence complementary to at least part of said target nucleic acid sequence; and
(b) at least one nucleic acid sequence comprising at least one gRNA that targets at least one protospacer within said target nucleic acid sequence, or any nucleic acid sequence encoding said gRNA, said gRNA guiding at least one PEN to said target nucleic acid sequence in said Fas gene.

22. The method according to claim 21, wherein at least one of:
(a) said splicing modulating agent comprises at least one oligonucleotide, said oligonucleotide being ASO, and/or SSO, comprising at least fifteen contiguous nucleobases complementary to at least part of said at least one target nucleic acid sequence that participates directly or indirectly in at least one splicing event,
(b) said SSO and/or ASO comprise nucleic acid sequence complementary to the nucleic acid sequence of intron 5/exon 6 and/or to exon 6/intron 6 of the Fas gene;
(c) said ASO and/or SSO is chemically modified.

23. (canceled)

24. (canceled)

25. The method according to claim 21, wherein said PEN comprises at least one CRISPR/cas protein, and wherein said splicing modulating agent comprises:
(a) at least one nucleic acid sequence comprising at least one gRNA, or any nucleic acid sequence encoding said gRNA; or any kit, composition, vector or vehicle comprising said gRNA or nucleic acid sequence encoding said gRNA; and optionally
(b) at least one CRISPR/cas protein, or any nucleic acid molecule encoding said Cas protein, or any kit, composition, vector or vehicle comprising said CRISPR/cas protein or nucleic acid sequence encoding said CRISPR/cas protein, optionally, wherein said gRNA targets at least one protospacer within at least one of: the 5′SS of exon 6 and/or the 3′SS of exon 6 of Fas gene.

26. (canceled)

27. The method according to claim 16, wherein at least one of:
(a) said cell express reduced levels of membrane mFas,
(b) said cell is further engineered to express at least one receptor molecule, said at least one receptor molecule comprising at least one target binding domain specific against at least one target antigen;
(c) said at least one cell is characterized by at least one of: (i) reduced expression of mFas: (ii), increased cytokine secretion: (iii) increased expression of activation markers: (iv) increased cell survival and/or cell viability; (v) increased cytotoxicity; and/or (vi) reduced expression of exhaustion markers;
(d) said subject is administered at least one non-naïve cell of the T lineage manipulated for enhanced skipping of exon 6 of the Fas gene, and wherein said at least one cell is of an autologous or allogeneic source; and
(e) said subject is administered at least one splicing modulating agent, and wherein said splicing modulating agent comprises at least one of:
(i) at least one oligonucleotide comprising a nucleic acid sequence complementary to at least part of said target nucleic acid sequence; and
(ii) at least one nucleic acid sequence comprising at least one gRNA that targets at least one protospacer within said target nucleic acid sequence, or any nucleic acid sequence encoding said gRNA, said gRNA guiding at least one PEN to said target nucleic acid sequence.

28.-33. (canceled)

34. A method for improving activity and/or survival of at least one cell of the T lineage, the method comprising the step of contacting said at least one cell with an effective amount of at least one splicing modulating agent comprising at least one nucleic acid molecule or any vector, vehicle, matrix, nano- or micro-particle or composition comprising said at least one agent, said at least one nucleic acid molecule targeting at least one target nucleic acid sequence that participates directly or indirectly in at least one splicing event of the Fas gene, thereby enhancing skipping of exon 6.

35. The method according to claim 34, wherein at least one of:
(I) said splicing modulating agent comprises at least one of:
(a) at least one oligonucleotide comprising a nucleic acid sequence complementary to at least part of said target nucleic acid sequence; and
(b) at least one nucleic acid sequence comprising at least one gRNA that targets at least one protospacer within said target nucleic acid sequence, or any nucleic acid sequence encoding said gRNA, said gRNA guiding at least one PEN to said target nucleic acid sequence;
(II) said cell is further engineered to express at least one receptor molecule, said at least one receptor molecule comprising at least one target binding domain specific against at least one target antigen; and
(III) wherein the method results in an improved cell characterized by at least one of: (i) reduced expression of mFas: (ii), increased cytokine secretion: (iii) increased expression of activation markers: (iv) increased cell survival; (v) increased cytotoxicity; and/or (vi) reduced expression of exhaustion markers.

36. (canceled)

37. (canceled)

38. The method according to claim 34, wherein the step of contacting said cell/s with said at least one at least one splicing modulating agent, or any vector, vehicle, matrix, nano- or micro-particle or composition comprising said at least one agent, is performed in vivo, in vitro or ex vivo; and wherein one of:
(a) wherein contacting said cell with said at least one splicing modulating agent is performed in vivo in a subject suffering from at least one pathologic disorder, said contacting step comprising administering said subject with an effective amount of said at least one splicing modulating agent or with any vector, vehicle, matrix, nano- or micro-particle or composition comprising said at least one agent: optionally, said at least one pathologic disorder is at least one of: a proliferative disorder, an inflammatory disorder, an infectious disease caused by a pathogen, an autoimmune-disease, a cardiovascular disease and/or a neurodegenerative disorder: or
(b) wherein contacting said cell/s with at least one splicing modulating agent is performed in vitro or ex vivo, thereby obtaining a non-naïve cell of the T lineage manipulated for enhanced skipping of exon 6 of the Fas gene, or a cell population comprising at least one of said cell, wherein said non-naïve manipulated cell of the T lineage predominantly expresses sFas, optionally, said cells are of autologous or allogeneic source.

39.-44. (canceled)

45. A splicing modulating agent comprising at least one nucleic acid molecule or any vector, vehicle, matrix, nano- or micro-particle or composition comprising said at least one agent, said at least one nucleic acid molecule targets at least one target nucleic acid sequence that participates directly or indirectly in at least one splicing event of Fas gene.

46. The splicing modulating agent according to claim 45, wherein at least one of:
(I) said target nucleic acid sequence is comprised within and/or comprises at least one of: 5′SS of exon 6, 3′SS of exon 6, exon 6, at least one intron located upstream and/or downstream to said exon 6, and/or at least one splicing junction flanking said exon 6 of the FAS gene;
(II) said splicing modulating agent comprises at least one of:
(a) at least one oligonucleotide comprising a nucleic acid sequence complementary to at least part of said target nucleic acid sequence; and
(b) at least one nucleic acid sequence comprising at least one gRNA that targets at least one protospacer within said target nucleic acid sequence, or any nucleic acid sequence encoding said gRNA, said gRNA guiding at least one PEN to said target nucleic acid sequence in said Fas gene;
(III) wherein said splicing modulating agent comprises at least one oligonucleotide, said oligonucleotide being ASO, and/or SSO, comprising at least fifteen contiguous nucleobases complementary to at least part of said at least one target nucleic acid sequence that participates directly or indirectly in at least one splicing event;
(IV) wherein said PEN comprises at least one CRISPR/cas protein, and wherein said splicing modulating agent comprises:
(a) at least one nucleic acid sequence comprising at least one gRNA, or any nucleic acid sequence encoding said gRNA; or any kit, composition, vector or vehicle comprising said gRNA or nucleic acid sequence encoding said gRNA; and optionally
(b) at least one CRISPR/cas protein, or any nucleic acid molecule encoding said Cas protein, or any kit, composition, vector or vehicle comprising said CRISPR/cas protein or nucleic acid sequence encoding said CRISPR/cas protein; and
(V) wherein the splicing modulating agent comprises:
(a) at least one SSO comprising the nucleic acid sequence as denoted by any one of: SEQ ID NO: 2, 4, 6, 8, 10 and/or 12, or any variants and derivatives thereof; and/or
(b) at least one gRNA comprising the nucleic acid sequence as denoted by SEQ ID NO:15, or any variants and derivatives thereof.

47.-50. (canceled)

51. A method of enhancing expression of soluble Fas (sFas) and/or inhibiting expression of membrane FAS (mFas) in a cell of the T lineage, the method comprising contacting the cell with at least one splicing modulating agent comprising at least one nucleic acid molecule, as defined by claim 45, or any vector, vehicle, matrix, nano- or micro-particle or composition comprising said at least one agent, said at least one nucleic acid molecule targeting at least one target nucleic acid sequence that participates directly or indirectly in at least one splicing event of the Fas gene.


SEQUENCE LISTING (55 sequences)

SEQ ID NO: 1 - DNA, 113 bp, Homo sapiens
  source 1..113: mol_type = genomic DNA; note = FAS DNA coding strand sequence (intron5EXON6intron6 | target site)
aaatgtccaatgttccaacctacaggatccagatctaacttggggtggctttgtcttcttcttttgccaattccactaattgtttggggtaagttcttgctttgttcaaactg

SEQ ID NO: 2 - RNA, 20 nt, synthetic construct
  source 1..20: mol_type = other RNA; note = T=U; note = SSO No. 1
ggatcctgtaggttggaaca

SEQ ID NO: 3 - RNA, 20 nt, synthetic construct
  source 1..20: mol_type = other RNA; note = FAS RNA target sequence for SSO1; note = T=U
tgttccaacctacaggatcc

SEQ ID NO: 4 - RNA, 20 nt, synthetic construct
  source 1..20: mol_type = other RNA; note = T=U; note = SSO No. 2
gatctggatcctgtaggttg

SEQ ID NO: 5 - RNA, 20 nt, synthetic construct
  source 1..20: mol_type = other RNA; note = T=U; note = FAS RNA target sequence for SSO2
caacctacaggatccagatc

SEQ ID NO: 6 - RNA, 20 nt, synthetic construct
  source 1..20: mol_type = other RNA; note = SSO No. 3; note = T=U
agttagatctggatcctgta

SEQ ID NO: 7 - RNA, 20 nt, synthetic construct
  source 1..20: mol_type = other RNA; note = T=U; note = FAS RNA target sequence for SSO3
tacaggatccagatctaact

SEQ ID NO: 8 - RNA, 20 nt, synthetic construct
  source 1..20: mol_type = other RNA; note = T=U; note = SSO No. 4
cttaccccaaacaattagtg

SEQ ID NO: 9 - RNA, 20 nt, synthetic construct
  source 1..20: mol_type = other RNA; note = T=U; note = FAS RNA target sequence for SSO4
cactaattgtttggggtaag

SEQ ID NO: 10 - RNA, 20 nt, synthetic construct
  source 1..20: mol_type = other RNA; note = T=U; note = SSO No. 5
aagaacttaccccaaacaat

SEQ ID NO: 11 - RNA, 20 nt, synthetic construct
  source 1..20: mol_type = other RNA; note = FAS RNA target sequence for SSO5; note = T=U
attgtttggggtaagttctt

SEQ ID NO: 12 - RNA, 20 nt, synthetic construct
  source 1..20: mol_type = other RNA; note = SSO No. 6; note = T=U
aaagcaagaacttaccccaa

SEQ ID NO: 13 - RNA, 20 nt, synthetic construct
  source 1..20: mol_type = other RNA; note = T=U; note = FAS RNA target sequence for SSO6
ttggggtaagttcttgcttt

SEQ ID NO: 14 - RNA, 20 nt, synthetic construct
  source 1..20: mol_type = other RNA; note = T=U; note = SCR
acaccaactaatacgaatac

SEQ ID NO: 15 - DNA, 20 bp, synthetic construct
  source 1..20: mol_type = other DNA; note = CRISPR_AS guide sequence
aagttagatctggatcctgt

SEQ ID NO: 16 - DNA, 96 bp, synthetic construct
  source 1..96: mol_type = other DNA; note = CRISPR_AS sgRNA full sequence
aagttagatctggatcctgtgttttagagctagaaatagcaagttaaaataaggctagtccgttatcaacttgaaaaagtggcaccgagtcggtgc

SEQ ID NO: 17 - DNA, 68 bp, synthetic construct
  source 1..68: mol_type = other DNA; note = FAS DNA long target sequence: (target site) intron5EXON6 sense
tgttccaacctacaggatccagatctaacttggggtggctttgtcttcttcttttgccaattccacta

SEQ ID NO: 18 - DNA, 20 bp, synthetic construct
  source 1..20: mol_type = other DNA; note = FAS DNA short target sequence:
acaggatccagatctaactt

SEQ ID NO: 19 - DNA, 21 bp, synthetic construct
  source 1..21: mol_type = other DNA; note = CRISPR_AS guide sequence
gactgcgtgccctgccaagaa

SEQ ID NO: 20 - DNA, 97 bp, synthetic construct
  source 1..97: mol_type = other DNA; note = CRISPR_KO sgRNA full sequence
gactgcgtgccctgccaagaagttttagagctagaaatagcaagttaaaataaggctagtccgttatcaacttgaaaaagtggcaccgagtcggtgc

SEQ ID NO: 21 - DNA, 138 bp, synthetic construct
  source 1..138: mol_type = other DNA; note = FAS DNA long target sequence: (target site) EXON3 antisense
catgtccttcatcacacaatctacatcttctgcatttggaagaaaaatgggctttgtctgtgtactccttcccttcttggcagggcacgcagtctggttcatccccattgactgtgcagtccctagctttcctttcac

SEQ ID NO: 22 - DNA, 21 bp, synthetic construct
  source 1..21: mol_type = other DNA; note = FAS DNA short target sequence:
ttcttggcagggcacgcagtc

SEQ ID NO: 23 - DNA, 20 bp, synthetic construct
  source 1..20: mol_type = other DNA; note = CRISPR_SCR guide sequence
gtattactgatattggtggg

SEQ ID NO: 24 - DNA, 96 bp, synthetic construct
  source 1..96: mol_type = other DNA; note = CRISPR_AS sgRNA full sequence
gtattactgatattggtggggttttagagctagaaatagcaagttaaaataaggctagtccgttatcaacttgaaaaagtggcaccgagtcggtgc

SEQ ID NO: 25 - DNA, 21 bp, synthetic construct
  source 1..21: mol_type = other DNA; note = Forward primer sequence FAS-FL F:
gcaacaccaagtgcaaagagg

SEQ ID NO: 26 - DNA, 22 bp, synthetic construct
  source 1..22: mol_type = other DNA; note = Reverse primer sequence FAS-FL R:
tcctttctcttcacccaaacaa

SEQ ID NO: 27 - DNA, 22 bp, synthetic construct
  source 1..22: mol_type = other DNA; note = Forward primer sequence sFAS F:
gtgcaaagaggaagtgaagaga

SEQ ID NO: 28 - DNA, 22 bp, synthetic construct
  source 1..22: mol_type = other DNA; note = Reverse primer sequence sFAS R:
accttggttttcctttctgtgc

SEQ ID NO: 29 - DNA, 20 bp, synthetic construct
  source 1..20: mol_type = other DNA; note = Forward primer sequence GUSB F:
agagtggtgctgaggattgg

SEQ ID NO: 30 - DNA, 20 bp, synthetic construct
  source 1..20: mol_type = other DNA; note = Reverse primer sequence GUSB R:
ccctcatgctctagcgtgtc

SEQ ID NO: 31 - DNA, 53010 bp, Homo sapiens
  source 1..53010: mol_type = genomic DNA; note = FAS DNA NCBI Reference Sequence: NC_000010.11
tatttttttattttattctataatttgtgtattttgttatatattttagtttactttagtttcatatatttactttagtttaatatattttagtttagtttcacatattttgcattgtatgggaggaaaaaagaattctctctctattcttccgaattcttatctggaacccctgcaaaaaaaggcagatgaaaaagaaaaaagcgaacagaagtttattaacaaataggcttcacgtatatatgggagatacccagggaatgagcaattctcaaagaagtggcttagaactctggtttatgtggcatcatcaacaaagaacaacacattggtggggaagtaacaagacaaggggaaaggatcttgagtttctaggggtggcaaattgtgggaaaacaaatacatgggaaactaatgatagacaaaaggttatatagtaaagtctgttatgtagatttccctgtgccatcttcaggcaagcaagggcctaaggtcatccctggtgatcaacctttgtccttcttggtagagagggaaggagggacacctttataagtgtatgtcctgcttttaagcaaataggggtagggcagagagattttttgtatctacttctcgattgctttcaacccaaaataatccttatgccagagaggcatattttggggtggcatattcagctacccttcagcaaaaagtatctacatatcttgtaccaactggtctccttccctgtttgcagctgtacaaattaaggtattatgaaagcatggtcattcaaaggaggcacccgttctgctgctcctttgtaactttcttttgcccgcctgtttttcctctgccctcctgtttgctgctcctttgcaactttcactccaagctttacaaacatttttagtgtcttaagtttcctccttccccacaacacacaaattcatatagtccagccctgtgtattctatttccagagccggggatagcttcccttttcttcttctgccaaatcatgtttctgccttattttaaaagtctactcaaaattcatctacagcaaaccttcttagatgcattttttctacagggagaatgctccaatcactccagtgatttcatcagcacacttacttgatgtaagctcttatattttctcacatttgcagtatattcaccattaggttgtaaacttttggaggcagccagtgtgttcatttttcccttagggcaccctgcccttctacatatagtagaaactcaggaatttggttaagcaactttctattttccccaaaccttaaaagagctgtctgcaatgaccatctcagacgttgttagtttcagaaagtttccctaatttattttaaggaaagaacagataagatgcataagataagggacattctctggctgtaggctttctttagcacatttcaggattattataacataaaagccttgccctattttccttaagcagcacgcggctgcctagtgtacttgccttaggaggtcaaacacattaggtataccaacatatcgaccaccaaaagccaatgcacagaacagaggtcctcgggcaaacaggttgaaccatagttgcaaggtcaagccattataatgcaaagcatccaagggtgaccatcatgctttcccctgcgtttctgcaaagcacagattttcccagggattgctgagcaaagagccccttactaaagaaacagcagactccatggaagagtgtcaattttatgatccactgctggtttgggctgatttgcttttaactaattattgatcaccgtaatgactaacttttgagagagccctgccaaaaataaatattccttaaatgaggtacaccaatgtttttttccactccagtccttgaaaatggtgagttagagcagagtttcctaaagcatggcctgaatttacggaaccatggtcaacagaatcacttgggcaatttgttaaaatgtattaggttggtgcaaaagtaatcacggtttttgccattgaaagtaagggtcctaatacattcctggggctcacctgagccaggatttctggcatggggcctagagatctgcatttaaacaagagccacacagaagcacttggacagagggtaagaagggaatgtgatactgaagtctcaaaaatacagaagaatagtaaggaatgatcttcatcaatattatcatagctttttcttgttagattataggatcccaggcttatgtcctcagagaacagaatggagagcagatttatagacagggaaggggattggacaggactgtgggcctgcattctattttaggccctgcctctgattgtctgtgtggctttgaagagttcactaagccctttggccctcattttgtaatctataagtgtgtgggcctattattttaatatcctgtctacctctaacaatttctgattgaaatattgaatttgaaaatataaataggcagtataaactcttctgtttatctgtggcgtctgtgaggtaataagcagaagaggttgatagtttcacaaaaggaaagcatgacttagacagagtgcctaaaattctgtcccgggaaccatgatctcctcatctactggctgtctgtgtttgggcaatcacctgcatatcagtttcctcatctgcaaaatgacaatggcacttgccagccaccacttttcagggctcctgagcaggtcaaagacgtaacctgctcaaatgttttgaaaactattaagtgccaatcagattatcagttgtcactcaaatttggattaattaggtcagatattaggtgtcaggtggtgtttaaatccttgttgtccagagtgacatgggaagcactggaagcttaggataccattaggagatcctgaggcttgagtgagaagatcccataaagagcaacacggctcaaacagcgccaagagggcaggctgtgtgccaagatcagcctgggagtttatacaacacttattttagcttcagacatactttagcccctagaaaagaaagtgaacaggaagagaccctcgagatatctttggatgcatcaagaattttaaaagctgcaacttggaattcaaagcattcacttccttttccaacaaaattgagaccctatagggtaattttatactgaagagctcccagttctttaaacctgattggaatttgagatagttggaacaggacaagactcttattacctatgataaaaggcatcatcaggtaggggcaagagtatggactctggaggtaggttggattcatatcccagttttcaacaatttattttgagtcccttggcaaatcatctagcatctcacctcagttttttcatctataaaatgagaaggatgtaacacctgcttcatcaggtggcgatgaggacagatgcgagaatacagtggaatactgaattctggtgactttcaatattctattcctcagcactaacccatgcttacatgcattttatgacaatccacgttacctaagcccaaattattacctgcaacgcagcttggcaaccttcaaacactaaccaaatcaggtatcagagcccttgtttcttgaactttcaaagacagaagatgaacatataaaatagaatattttgtttcatgattttgttactgtaagccatgtctggtaaagaccaatacaaggaaaatgggctgaatctgtgaattacatcagcaggttcagaataaatgatggctcacacaccagaactcatttcaatcatccctaaatttaaaacaggaaaatggttggcaaagttgttccctttcttcctctggagaaactacttgaaataaatatgaatttgaattttacatgaatgatatctcaagagtttgttagtatgattatcatacttcaagtttatggcttatccagaaatcaaacaactatagtaaccagataaaagtccttttttttttctgtaacatataacagtaattagagttttctacttgtgaccttaaggtgtgttgtcatggtcttgacatttctgcagaagtaaactattcaattcagtagtttctgtgtttgcagttatatgtatttaacttgtattttgcatatttaatttgtatttaaatcgctaaaaactggtaactcagaaatgttacgtatagacatcctcatttatccagaatatttgactaaccagacacctaattatgttttgagtggtcctatctatgcatttaatgaatgttcttattttgtagccagcatatgtaatacaggtgattcctttcagcgatgggagctggacaaatctatatgcagggtttttgatcatcttgggtttaaaactcataatggagcaattaaaaccatgtttgttttcctgaatccatgacaacaataactttatcccaaaaccagctgtgaatgttggtaacacgggcactctgctgacctgaaggaatgcaggcacagtgcattttgcacacatgtttgtcggtagagtacttttttggtagagaggaagagaaaattctggcttctttagagaagtggatgtaggcaatccctcctccaaaagtagtcaaaataccactccagagaaccttgactttataagggaaggcagcttctgaatcactgaaagccttttcctccttaattctcactggctggcctggaatgaggctctgctaaaaatgttagaggcagctgttgatataggcatttctgtcctcttttctagtaccataatgtgtgcatgttaaagagattctgcaggtcccttataccttattcatcccttttaaagccttcttcttggaaagtagatgtggaggatctagaattggaaaagtttctgtctttcagaactttgtgtaggtttggtattcataaaacattacattatttgatgtctccccccaacccccaccccgcaatactacacagagatgaaaaatgtgtttaggtagaagatcctcctaaataagaaggaagggtctggaaagctgttatctgggttgtattacaactttgtgtttttttctctcttattaaataatactcaagacaaacacttttataagtaggatcttgattattgaggcaagaagtataatcttaagcagtgagaatgggggaaattggaacacattgttattcccataggatcagcatagatttccgtaatgtccaaattaccttgccatttctttcatttcccaaaaaaagctctttagtgtcaattttccatcaaaaaaaaatctttggaagaagtgagtatttccacattctgctatgccttagggcatcagctgtgcccaaactcatgcttgtaaggctgaatggtcactctgcttttgatgtctctatttgaaaaagcctacaaatccaaagaaagcatgcccccattggcatagtctctgttgtgcaaaggagactttcagagctttaggctccatctgtcatgatcatttttcaaacagactattttttagatcagttttatgtttgcagcatcaagatcacttttttttttggctggtttactcctactcaaattgtttctggctctctgggatgaccagtttctcaaggaaagaaacctcctctgaccaccatatcatcctcccaactatatcttttcatatacgttaatagcactctatctttccttcatagtacttactgtagtttgtagtaatacatttgatacaattatttcattcgtatacatcttcctcaccagcctagggaagggacactgattgttcaccatcatatcccagtatctagcacagtgcctaagggataggaggggctcagtaaatatctcttgcaaggatgttgttaaattgtaaactctgccaaggcctttatgttgaatgaatgaatgaacgaatgaatgaatgcctctaactcttcataaaataaacactttttggaaaaaattgctcatggcagacgtcagatgtaaccacactgataactggaaagaaaaatgaatttagatgggttttagagatagaacaatgtttctaatattcaagcagtccataaatcccttatacttaatgttaaaattcaggatatgatacctaaagaaaggttttactgtacttggatcaaggcacctaccaactttttctctactcataggcaaagtcactaccctgtttaaggaggtctttggacgtttccatctcaagaaacagctcttcaatctctcccttgtattccaccttccagaaccacccagaaattccagtaaagtcttattgacctgattgtctgtcctcttcgctgagttccttttgaatgcagcaaattgctgtgcccttttctgacttccatttcacttttatccagcttcataggagcttaggcccctgcttgttgaggagggcgttcagttagtgctgagactacctgaataggtctggttctagctactcacaactcagagaggtagtgaatgctaggaggtaggaagaaggacattcaaccagaatactaaaattgcatttagatagtccagaaattagacagttggcacagcattagaattccccatttatccttacaatagtttgctgagaatgatggttgggaattgaacaatgagaacacttggacacaggaagaggaacatcacacaccggggcctgttgtggggtggggagagcggggagggatagcattaggagatatacctaatgtaaatgacaagttaatgggtacagcacaccaacatggcacgtgtatacatatgtaacaaacctgcacgttgtgcacatgtaccctagaacttaaagtataataaaaaagtgtgtgtgtgtgtgtgtgtgtgtatatatataaagaattccccacttaatatttatacatccaaaaactgacaaaaataatgtctgaactcatgtctgacattcagattcctccgcccaaaggcaataaataaaataatactaagatgtaaaggcaaaccttgtcttctgtcatgatctacagatggcacaaataatagacattttcccgaataatggcgtctgattttcaagcaatgcttagccaattgtagatttgggcagaaaaagtttgtaccaagcatttgagctggaaaaggcagatatttttcttgacctcacttttcctaaagttcacaacttctgctacttaaatgagtatctcatctctaagtgtgtgaagtaacagatgatgtatctgtttttcatcctcaaagagttaacttgataaaataagtacagctaaggacaaacacaaacacaaaatgagaaaacttcatgcagtatgttttgtgttttgtaaagggtgccaatggcatggagccatctgattctgttgcagtctttggcaacacggcccatttaaattcaagttaggatctgagtgtttgggtaaacagaataagctaagaatgagtggcagacatatttcctcaaatcccatctgatacaggtttagatccagattttgtcatttgactaactccttccttttttagatgatttggatttgcctttttttcttataataaaagtcataatgccaaccactttaagagttcttttgctgctcttttttgttgctatctttttgtaactaataggacacgtgtaagactataatacaaacaggagtcctagcatgtaatggctttagcaatttgtgcaatatccctctttttgtccttcctgcatattttctccctagaaagcagccaagaattggtgttcagagggtgatgctctgatggtgagcagatctacagtttgcctaacagtttatttaagccaaatttatagtttctgaagcgctagaaggctgctgtccaaggggactacttttttttttttttttagatggagtcttgctctgtcgccaggctggagtgcagaggcgtgatctcagctcactgcaacttctgcctccccagttcatgctattctcctgcttcagtctcctaaggagctgggactacaggctcacaccaccacgcccagttaatttttgtatttttagtagagacagggtttcaccatgttggccaggatggtctcaatctcttgaccttgtgatccacccgcctcagcctcccaaagtgctgagattacaggcatgagccaccacgcccagccagggactacttattaactggccatttaaaatgaaatgaaacaaggtcctcaattgctaacgattaaggggagaggagtgtaaattcagctattgtcttgtcttcaggcagcaggaaattaagggattggtaactgttatttgtaatagtaattgatgtcacaaaaggagttttgagaacagggagtttaggaaaaaaattgatctccttttatttttagtctgttaatatttaatgtaatatttgatacagttcgatttagatttactgttctttgatctatttttattttttgccttttttgttgttctctttcttctctcctgccttcttttatattgtttaattttttttctaaatttcattgtaatttatctattagctttttaactatatcttttggcattatttttacaaagttgctctagggattacaatatacatccttaaatactcaccatctacttcatggaaattactgtaaccttacaaatgtataaatcgattttacccttgttcatcccattataatgtatattataagcctcatacaatggtatgatttttactttttctctgcgttgtttctagttaactatatgtgctatcaaagtgagaccccatttatcttttaatctgtttctgattgtttttccagtgtctacttaactgagactcttacaggtaattttgtaggatactaggtgttttagatggcaaatataaagattgtgtttgtgtatgtgtatgtgtttatatgtgagggccaatctattgaaaagaaacactgcagaatactaaagcaaaattgaaaccatttagagtttgtagttgttttattttaaaagctaaccttgtaagtacttagtctttcaaaaaataattttaacttctatgttttatttttctattttaatttttcctgggccttgcctcccaatcctctcaccttccctttcttctgtgtgacctatagattcagaaattcctttctgggataatttctggcactgctttggagatggagcccctgaaaattggctatggaccaaatggattcccactcttggggatctctaggtcatcatcaccatctgaacagctctgagagagacaagaagtggaatggagaaggtgattaggtaatccaagaattgccaggcgaacaattgtgaaaatctttcatagagttcccgatgaaaacaactctttcttgttagccattatctctgcctttcctttctaaagaaaaacacgtcatttcatcttgattatcagtttctcaagtactcatggatgtgaggccaggtaccctgtcaccttggctctgtttgggatgcaatgttgtgtttgtcaccacttcccattccccaaaagcatctttaacatagaccattcattcttcagattcaaaatgcttaagctgaactaacctcaaagggccctcattaatttacagacatttgaataacacccatattcatttctgctactgaaccaaagccttttccaaaaaaatcatctagcaaactgtttttgccaggccaaatattaacaggtatatatatttgcatacataaatatttacctctatgtcttgtgtgtttttgttatgagacagagtctcactctgtcgcccaggctggagtgcagtggcacagtctcagctcactgcaacctccacctcccaggttcaagcgattctcatgcctcagcctcccgagtagcgaggactacaggcgtgcaccaccacgcccagctaatttttgtatttttagtagagatggggttttaccctgttggccagggtggtctcgaattcctgacctctgatgatctacccgcctggacctctcaaagtgctgggattacagacatgagccactgtgccctgcctctgttttgtgtatttacataaatacatgtctccagagctttaaattcttcttaaattttaaaactagaactaaaccagacattgagaggacccagaaatataccctgactaacgtgggtggaatttcatttggcttgactctttggtgaaaaaaaaaaaaatcaggctctcaaggactcaatttggtgaaccaatgccaaatagaaaaatgttcttgacattttccccaaaatagatgtctaaagattttgcacttcaaattggtactttatgtattaaaagtgatattgaaactaaattcctacccaattagttgaaaaatatgaaaaaagcaattgctagagaatgcaatttattaaatgttggtggtcttttaaaaaatgaattcctcttgtagccagtgagatcagttatttatttatttattttaatagtaacaaggtctcactatattgtccaggctgatcttgaactcctggcctcaagctgtcctcccatctcagcttcccaaagtgctgagattataggcatgagccaccatgcctggctagttaatggttattaagtaatacaaacatttctgtgaattccaccagatcattgactttggcatagttcacttaagactttgtttgataaaataaatctctttcactctaattaggaagcatttttgggatcctgattaatgctctaatattgtggggccaaaaaatatgtggaacgtggccctgcagttagatgagctgagatcactatagtactatatagcgctctaaatcctaaatgctagatcactgtaatactgtacagatcagccacttgctggcttgtgactctgggccatttacttcacctctcaaaaggcatatttaaagtgaggaaaataataagtttattttggaaattcagttagatgatgcaaattgtttactgtaatttctagcatgtagttcgtgctccatgcatttaactgttattacaggttgtattagtatcacattaaattacccactgaagtgaaagtgaaaccagaatgctgtcaacgattaaaaaaaaaaaaaactctcaagcaagagcaaacaatgatactgcctctacattttccaaatctttgaatgcttggctattttaaggtttgtgaggaaaagattgctaaatttatacaaaaggaaggagataaatcagaggcggaagaaaccgtgtacagagcagcgtgtgcatttgaatgaggtattagctccatgcagaaaaattagttgagaaggacaatagtactgtcatactttttaaaaactgaaagtacctgaaagtgtttgtttttttcttattacagactagacataataacaattttcctgtcattcctgctttcccagttctccaatacgtaaagaatcaggtggaacagtagagatggtacaggaaggggcatggagagcaaagggaactatcagcaaataaatgtgtgggctctgggcttaggaaaatggggccctgactacttttccactggatcgtaagttccatgagagtaggatttttttttttcaatgttggtcaatgctgcgcctccaggtcataaaacagtgcctggcacatattggcctcttaataaatattcgagaattaaagaaaggccacaaaccaggagatacttgacagagcaggaggtgacttggtgacttgagcttttgaggctcacctccattgaatctgtcctctatgcttagattcggggagtttttagtgcactatcagtatacagtactactcccattgtgttataccataatactatgcagtgagattaatagaagccaaatttctttcctcttcatttggctgacaaattaattgtcaccaaaatctgatgtctctttcaagtttgaggcaaggtcaaatgacccagccactgaatcttatgtcccatcccgattaggacccaaccatacattactattatcaattagttatagtgataatataatgtgtaatacaggggtgtccaatcttttagcttccctgggccacattggaagactaagaattgtcttgggccacacataaaatacactaacactaactatagctgatgagctaaaaaaaaaatcgccaaaaatctcataatgttttaagaaagtttacgaattcgtgttgggctgcattcaaagccatcctgggctgcatgtggcccacgggccgtgggttggacaaggttggtataatagataacatataattaatatattgatattatcacaaatgccagagtaaacaaggggaaactatttagtctctaacaaaaggattgataggccaaggaaaatatattggttcattatgaggatcacggaaaatggagaaaattagtacattaattacatttgaatagtccttccattgtactgaactaattcaagtgttttgaccctaaaattttgtagttctatagcaaaaacccacttcctttccactaaattgaaaatgactaacatatctctaatgtaccctgaacataatcagcagcaattcaatgatattgtgttattttaaaatacacaccaactattgagacagatgattataattttaaattaacaaaattgtattcataattaagaagctatgatagaaataaagtgttgaacagaatttcttgaaagtatttttactaaataaaaccaaatgtaaaggtccaggtgctgggtagagttgaagtttgaaaattgaataaaataattctttcattaaatattattagagcttcttagaggctcaaaccaatgctgatgtttgtagagaagttgcattgtgcttgagatccttatatctggtgaaggtaatttttgcccacattccccagattcctttagattagcagcaaaatgtatgcctacatatcccaaaggagaagcttattttctgctattttgacatctaatgaattaatgcttgtgtggactgaattgttaaaaaatttaagataattttctcccattttgtaggttgcctgttcactctgatggtagtttcttttgctgtgcagaagctctgtagtttaattagatcccatttgtcaattttggcttttgttgccattgcttttggtgttttagacatgaagtccttgcccatgcctatgtcctgaatggtaatgcctaggttttcttctagggtttttatggttttaggtctaacatttaagtctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatagttgtagatatgcggcgttcaacctactcatctgacaaagggctaatatccagaatctacaatgaactcaaacaaatttacaagaaaaaaacaaacagccccatcaaaaagtgggcaaaggacatgaacagacacttctcaaaagaagacatttatgcagccaaaaaacacatgaaaaaatgctcatcatcactggccatcagagaaatgcaaatgaaaaccacaatgagataccatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactggaaataccatttgacccagccatcccattactgggtatatacccaaaggactataaatcatgctgctataaagacacatgcacacgtatgtttattgcagcattattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaatcatcattctcagtaaactatcgcaagaacaaaaaaccaaacaccgcatattctcactcataggtgggaattgaacaatgagatcacatggacactggaaggggaatatcacactctggggactgttgtggggtggggggaggggggagggatagcatcgggagatatacctaatgctagatgacgagttagtgggtgcagcgcaccagcatggcacatgtatacatatgtaactaacctgcataatgtgcacatgtaccctaaaacttaaagtataataaaaaataaataaataaataaataaaatttaagataagctcttgactgtccttttcacataagtgggtgaagttgagaaaacataactgtgaatacatttgttgtactctgctcagcctttcatcacccctgctctgacctgttggaaagtggtaagagagagaaaagattctgttctattgaaagccaatctcaatggaaagaagtgacaaccagtcaggctcatgctgagagatgggtctagtcatgagccaggctcagttcatttgcaccacggctccagctcaccctacttcagggaactgaagatgtctaggtgtctgggtcaccctgagagggggagtttattaaaatgaatcctctcttctacaaattcatgctaaattgttccacgtctaatggtaatttctccaaaaagccttctatgacacctgaagctattagaagcaaccttccttattctttctgtccttgaatttgatctcttctctattattattattatattattattattaataggaactaatattcatcaagaatttactgtttctggcactgcatagcatcttttctatgaattacctcctttcatcctcctaataagcatataaagtttattctgttaatatttgttttataaatcagaaaactgaaagctcagaaaacctttttatgtttaaaaacactgtccactgcctgaaacactgaaaacaaaatggataaaaattaagtacaattaaaattatagattctttagcttctactaagctggttttaatgggttatgatgttgtgtatgagcgtagggatctggggaggagagataaggaagggaagaaagtgagtactgagacaaaaaagcagaaaaaaaaaaatgaggtcagcgagtttcccggctctgccgcagggtgccttgccagccttggctcctaggaagggaagacttgtggctcaccacagaccagattcctgctgacttcaaagagagacgttcacaaggagaagatgccagcattcaggacagatgagagtctggccaaataattgtcaggaacccttaaaatgtaggctactcatcttgggatgattttaaacagtaataagtgtaggcagcaataaccctaatgaagtatggggaaagagaatgtgagagtctcacactagggctgttctctctcactctccctctcttttaattgttaatgatgactttttttttttaacaagtctacataacctgagagatggggcgagaacagcataaatgccatggttttgatcctcctatcaaaaatcaggacaaaatctaaactcaattcgatcagaaaaggttacatgtgcttgactgtataatttgctagccatttgtttagtcctgactctctaatcaaatgccaggaggatccagagagcaacagcaaaggtcatttctagtgcctgtgtcctcaccacacacctcctcacacatgtgtaaagcacctacaaaggctttggggtagatcctggcaaaatggaataatagaagatatagttcttacgttcaaagagcttccaagctatttatggagcttgacagatgcagaaaaaggcaacgctaatttgcaaagtctgtagtttcagaagatggccagctgagccgaaagataaagtggacttctttccacttgaaattttatttcatttattaatttttaagttttgttcaccacttctacttttcccacccttctggtctggacagaaatgaaatagttcagttttaggcacgataagaattcaaggtggtgagtggaacatgagcaggaattaaacataatgcactttaaatgtacaactaagaagtcagggcatgaggtatagatttgaagagcatctgggaagaggtgatggttgacacttcaggccttctttgaagctaggggacctgaggactgaggagagcacattagcaagcatcaggacgctgaacataaccacagtggaggtgacagggaagcaacagccagggataaataagagccagaactgtggtgtctggtgagccaaaagagagacgcatttcacaggcaaagagaggccaggcctgatgcttataattactctaatttgtcatccccagtaatctgtagagtccaggaaggcaaggaatgttcttatcctgcctactgctattccctgaggcctaacacagtccatggtacaaagtaggtactcgtaagtattcgtttagttaaggaaaattgttgggatcttagacttaagctcaaagcctccattcaaagaaagtggccaaaatgtcccccctctggagcaagttgcattctgattaggaatgagactcgagccatataaagtcaagtacagttacaaggcaatgataagaaagaatgaggtagctcaattccaaatgaatctggatttatctgatatgtaaatttaaaaagagagagataattttcccagactccaaatgccaaatcagtttcactccctcctcaaaagtgctgagaaagttctaaatttggaaaggtcacaaagggataaagtgagtcagaggaggctttggtttgccttatatgaaaagtgggatttggactatggaggaatagggtccttccagaggggatgtttgaatgacagagtaggaacagggcatggggaggccaactgcgaggaagtcagcacaagcaaagggcaggcccagcaatatccaagagagagcggggccagtccacagggtgttcagtgtggagcctccagacctgtgtcctaactcgagctctgccacatactcgctgtgtgcaaattagttaaactcacctaacctatgtttcttaatctgtaaaatgaggataatattagatgttaagattaggtgaggatcaaatgagacattgtgtgcataaagcacttttcctgtgcagttaaacccttgataaatattagccacttattaaccatcgggttaagagagaaatgactttttttttttaaagaactggccattgctttatcttccatcgtcgtgatttatactttttaactatattcacaccaggaaaagttgatagtaaatttcattttgggtgaggtctagactagtatccgtggaaaggaaggcaaattgaagaggtatgatcctaggacagaggaagaaccaaggtcaaaacagggacatttcccaattcaatccaacagctgctttgttaacagtaccacaagcagtggggtgggcagagtgggagacgcatcatatcctgggaagggaacactgtagggagggagagagaggctggatgggggaaacggtagataaaagattgggcaaagtaatttagtcccagtcccagatactacgtgtttcagtctgtgctcgtgggtatgtaaaggtttcagatgggctttccatgaaaaacagcaatgaggcaaaccgtcccattcactaaaaagacaggcattatcatgatactgtgcaaagagcccaggctttagagtcaagcaaatcttgatacctttacttgacacatgaacttgatcgagttacctctatgtgaatcaggaaagttattctttccctgtaggtgtgggtatgagaattaaataagttagtatatataaaagggtctaacacatagtgagtgcttgattaactggtatgtgttagttcaagaaggcagtatgttgttatatgcgagaagcaattgctttggttcaaaccccagctctacagcttactggttgggtgactactgcaagatcctcaagtgcttgggcccatgtttgtttcacctatattgtgcacacaataagaagacctacctcccaagatttttatgagcattgaatgaaatcatatttataaaacccactaaatggtacctggctcatactaagtgatctaaatgaagaggtattatcattaagaagcaattaaaagttgcatgtgtatgaagttaacagcttttaacataaaagacaatggattatttggtaagagatctcaccgagcttaaatgtgtttttttctttttttttttgaaatgaggaaaggaagacataaatggcagaatatgtggtacagaactgtaagatacatgtcccacgacagaaataaaaaaggcagagattcactgaggggggaattcatggacatttatggtcacattatcctgattggctgagggagtcagcaggcttctagccagttttcttgctgttcacagaaataaaagctcagtgtctttgaaaatgaactaagtttttttttctttttcttgataggaaaatgtatttatgtagtcaagtaagacttgtttctgctaagtaaaaattcagcctaagcagtgtaatatgaagctaaatggagactggatacaaatatgtttataatgagattactgaatttgttgatttccctacatttcccatctactaccactaaagaaatgctgtaataataatataactggccaaagtggtaaaagtggggttaattggtccttatctgtttgaggtaaacaaccgaactgatgagtggtttccctgagcaaagactcttgctacttcatgtgtcctttccagaacgtggcatcaacatcacatgggagctggtgagaaattcataacctcatacttagacctaccgaatcagaatcttaatcttaataatattgacaggtagtttatatccatattgaggtttgaaatgcactgagttagagtgtggagttatggccacactgtcttaattagattatctttatttcaatccaatttgcatggctatgtgagctatgattcttagaggagtgaacataaacagcaaaaattcttacttagcagacttacccagtgcaagaaagagttcccttatctcactgtattatgatcttagtcaatatgacccggaatttaagaaaataatatgacaagagtatggttttaatcaaccggaaaccatgtgttcaacagaacaacaaaacaagaaggctttgtacttacttagggagtttacatcttgatgataaggccactgaggtaactaaagaggaaatggattcagcataggctttggaagttcacttctgttacctagtaactgtgtgactttgggcaagtctttccaagtttcttttctataaaataggatggtaacaggacctactttacagggagttgtaaggtttagataagttaacaggttatgcaaaaaaaaaaaaaaaaaaaaaaaaaaaaaacacacacacacacacacacacaaatgttctggacattatataagcattagatattactgatatcttatttttgtttatttttaaatgtctgtccctttaaaccattcctttcaacatatttgtgtggtttaaataaaaataagtttgaagaaatttcagaaagaactttgataaacccattgaatgacttggaaattccaaggggatttcaaagccaggtggaggaatgcctgcttagtgtctttcctcagtgcaaaggctgataaaggcctcaatttctccctcagtttactgtgatcagagacttaggctgcctagttatccttgttgcctggacaaaaaactctttagatgcatgcaggtttgtgtagagtttgtggggaggtttcagatggtaaaatgcctcttcctacctcaggtgaggggagggttactatcattcctttttcacagagctattctagttctgtttataagtagtgttctttgtatagtggatgttgagcaaacacttgttaaactataggaagttataacatcgggagaatttctaacaactgcaaagtgttctgatatgtaataaattgccttgagaaatagtaggagccagtgctggaaggagtcaagctaaggctggtgtagtctcctagggctgttgtagagaagagtctaaagtgggaggtgttgatcttatggtcccagaggtctctcccatactacctttctgcctgtataggtcagaatctattaagagtgagtaggaatggattacaccccacagtcatgaagggccttaaatgatagtagcacactagtgtcatatgtcaagtggcaaatgacactagcaccaaatgtgattctgcatttaagttctaagtattaaatgcatctatttgctatatgaaaacataagatatcccagtgtttgcaaactggttcaatattgtgttgatttttgttttgctttaatacactaagggaacttcaaaaggctgatgtggtccatgctctctgagccaggcctctcttgacttcttatgtttagttattcatgaaaccactgggcctgtctaaatggccaagtcatataggaagagacagagtgaaagggagagcaagagctagagtgagagaaagagagagagacatcttctcaccttcagacctgatcagaggcaataatgggagaccatgggaggggagagcctgatctgatgattccttttcagttattttctggaaagtttaaaaaatatggtattcttcatggagaagccaagatgttaggctgcaggcagtgcttatgctctctagcctagtgaggaggaaagcagtataggtaaataaaatgacatttagttggtaatataggcagggcctatatcaactgccacctataaaaggcgttggtgatggtctttagggggccattggcaaattgctacaacaaaataataaaataaataaaataacaaaatccaagtttatctcccagaatctatctgggcatatcattactgttggaaagacttatgtttaccaggtgagaaaactgatgctgagaggggttaaatattatgcacagcttctcacatctagtgaagtaactgaactaggatttatatctagggactctgtccttcataggacaagttgctaatttgggggtgttctcagggagaggaaaatgtctatatgctcatatgcacagattatccaaatcaaaactctttctccccattgtcctttctcagttgaactagatgttaataccactattgtattagtcgactaacattgccttctgcagaaaaccacctatgtctcctttctacccaagtgctctggggctcctcccctgcccattctcctccaacatcccattccacattctctttctatctctgcaggactccctgtagtcctccctgcccatccattatctccctcaagctgtcccctcagccttttctaacagggaaaggctggtgccctggtaaagagtcccccacccccatcccactccctggcactagatgggcttgggagacaatgactgcccctccctcatgctgttctgatgtctcttgttccttccagtcacctcccttccaggcccatgaggagggcctgaggcccacactagctagtcttttctctggaggttgtttgggggcagggaggaagtctaaagtagagagaaagcgctgccatgccttaagccaacctttccagaagtataatgcccttgccactagtagtttgggaaacaatttttattggcatgcaggcaaatattttgtttcaatagtggcatatttatttttatggataccttctatttttggcaaggaatgctgattttccatttatggttataatgtaatgttttattttaaaaacaaaatgacttagggaacaaaataagttgacttaaaaataaattattagttaaatatatatttagtatatacaagtagactacagaaatggcaaaatagattgaagttgaagaaacattgccttaagctaagcccactcaggatgaaattttatctgcccccaaatcaacatctcttgaaaagtggtccctatctttccttgtgtcagttcttaacacccagggggtaggcatgcttctcatgtttgttttcatagaatatctgtgttttcaggggaccaccatatgcaaaaatgtcaggggaggggtactcaattggattaagaagccagggcccacagaacagatgttttcaccctactaacccaattgctgtctgctttttagttgccacagtctaaacattctttaatgcattgtctttttcagtcattgctcaagaggtaggttagtataccagttttcatagtgggaaaaattaggagacttaataatttgctgcatatcttggtggccaagttttccagccctggtcagttaggctttaggacacttttcttaggcttcaggaatgtgagcatggaagtagtacagaaaggaagtagtacagaaaggaagtaatacaggaaggaagtagtgcaggaaggaagtagtgcaggaaggaagtggtacaggaaggaagtggtacaggaaggaagtagtgcaggaaggaagaagtataggaaggaagtagtggaggaaggaagtagtacaggaaaagtggttgtaagccatttaacttactttttttcatctgaactgtgacccccaaattatcaatttgtcttgtgaagtcacttcatttaggaaaagcaatgtaaaagtaaaaattagtatttattgtgtgtactaggtgatttgcatatgtcatttctttaatcctgtaggaaataatatgttctccaggaggtgacaatgtctatatgattagattcacagattacctatccaaatctaagatctccgtcctctctcttcctttcccaagaacccatacatatttctattaattggtagaaataattattaatagactttgtcacttggccactgtgggctctgacgccctctagtgacagaaaaattctttggtagaagaagtaatatccctagatagcagtctacagaattgtttaagcaattatctttacttgagcttgaaactagaatagctaatttgtaacaattaacttttgtaacaaaagaattattatactgatcaaagttctgataatttatcagttaagattttttcagttgtaagaatctagcttaagcagaaactaagaatttgtagagttgggtaactttgggtggtccagggttcaatttcagaaccaaatgcatccagaacttggatagctctaccaaccttgatctttcagcatggtttcctctcagtgtggtttcctctcagcatgagttgactgttttcagctctggcagggccacaagctcccttcacaaccaaccacatcagtttcacatctcacatatttctacttcaaatccaatgaaaaagaaagggtttgtgtcccagtgtttccagtaaagatcccaaaatctactccaactgaatgtgatgtgctgattggcttaagccaagtcacatttcccacccactacctccctccacctacacatatacccaagccctgggattggtaccaatgccactgtgatggttaattttatgtatcaaattagctaggccatggcacttggctatttagtcaaacactatcttagatgtttctgtaaagcaatgttttggatgggattaacatttaaatctttacactttgagtaaaacatttgctttctataatgtcaataggccttatccagttagttgaaggccttaaaaaagaaaaaaaaaaagactgacttccactgagtgagtgagaattatgccagcagacggcctttggacttgaactataactttttcatgggtctccagcctgttggccaatgctaaagttttggacttgccagcctctacaatcacgtgagccaactcctatctatctttccatttattcattctcttggttctatttctctagagaaccctgactaaaagaaccactcacatcaaatagttaagaagaggggagagggacagtttttctaaagacaacttggaagtactattaccagaaggaagaagaaggagaaaggcaggataaacagtcatggatatacactatagctgggagttattaaaatcaacttattgcaatttagtaaaaacaatttggttgacaacattttttaaacattaggaccaaaggggaaaagaacaaaagtcttagtggtaaaaagatgtagaagtttttttttttttttgttttgttttttatcttaactcttcctgtttttttacatagtcaagattcttgtttaagtgttattcagaatgttcagaaaccaggatatcttttgagtagaagctttaacttatatggtatggtttgaataaggatggttgtccaaaatatctccaacatctgttggagtgcctgaaattctattattaaaattttatttctttgcccttgaaggcctgaggactctcaggaatatgctggtaaaataaaaataacctttagagatgcccaaactgttttccccagaacaccagcattcattaggtgttcattcaatagattcttcaaaggattccaaaggcaaagaagtttggggaacagtatatataattacccaaccctttgacattagcatactaagggccctgagaagttttggattcagaaagttttcaaattaaagtaacccagaattttctaagattatttgaccatgaaacatatgtctccccacaaagcacatattcctatctccttgaacttgaggataattagacgtacgtgggcagagggtaggggaagggggtatggcatagaaagagcaggaccttgggagcaagaatatctaagtttaattcctgactctgctatttattaactaaccatctttgccaatgttgcttaagcttttttggctacatttttttatttgtaaagtaagtttaataatcactcatctcactgggctataatgataagtattaagtaaggaagatccacatatgtgagttgctggcttataattcacactcaagagatactgattttgtcaattgtcctttcccctttttttctctcttccctccttccattccttcttcccttacctctcctttccttccctcacaccccttttccttccttctttttacatttttttatttaaatgaacttttcattttggaatagttttaggatttcaaaaaatttgcagagataatacagagaatgcccatataccatcctccttatcccacttctttttgtgtctattagatgctcagagtgtgtgcacaaggctggcacgcccagggtcttcctcatggcactaacagtctactgaaaggtggaacagagacaagcctatcaacacctacaagactggtggtaagtgcagtgacagatgcaaaacacagggtgatggaaagccctcaggagggtaacctaacctagatttgagggcccaaacaggctccagaagaaaatgtcaactgagaggaagcctgaaggatgaacagtgggctaagcaaagggttattaatgtgttattaatgggttgaatctaattgggaagggagagaggttgcagagtgaggtgcagagcttggtggacgatgccaaaggaatactgaaacctttagtgtgtccagtctggaactgcatccaaattcaggttcagtaatgatgtcattatccaaacataccttctgtaaaattcatgctaaactacctaagagctatctaccgttccaaagcaatagtgactttgaacagtgttcaccagagcacgaaagaattacaagatttttttttaaagaaaattggccaggaaataatgagtaacgaaggacaggaagtaattgtgaatgtttaatatagctggggctatgcgatttggcttaagttgttagctttgttttcctcttgagaaataaaaactaaggggccctcccttttcagagccctatggcgcaacatctgtactttttcatatggttaactgtccattccagaaacgtctgtgagcctctcatgttgcagccacaacatggacagcccagtcaaatgccccgcaagtctttctctgagtgactccagcaattagccaaggctcctgtacccaggcaggacctctgcgctctgagctccattctccttcaagacctccccaacttcccaggttgaactacagcagaagcctttagaaagggcaggaggccggctctcgaggtcctcacctgaagtgagcatgccagccactgcaggaacgccccgggacaggaatgcccatttgtgcaacgaaccctgactccttcctcaccctgacttctccccctccctacccgcgcgcaggccaagttgctgaatcaatggagccctccccaacccgggcgttccccagcgaggcttccttcccatcctcctgaccaccggggcttttcgtgagctcgtctctgatctcgcgcaagagtgacacacaggtgttcaaagacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggtgagccctctcctgcccgggtggaggcttaccccgtcttagtcccggggataggcaaagtggggcgggcgcgggacgcgtgcgggattgcggcggcagcggcgcacgcgggcacctgggagcggcgggctgctgcgggaggcgttggagactggctcccgggggctgttaggaccttccctcaggcccgggtgctcagaacgctggaggacttgcttttcttgggccttgatgcgaagtgctgaccccgctgggcaggcggggcagctccggcgctcctcggagaccactgcgctccacgttgaggtgggcgtggggtgcggacaggaattgaagcggaagtctgggaagctttagggtcgctggagggggaccccggttggagagaggagcggaactcctggacaagccctgacaagccaagccaaaggtccgctccggcgcgggtgggtgagtgcgcgccgccccgcgggggcggggagagagcctgcagccttcagaacagatattgctcattttctggcagttctcagacgtaggaaataagtcagcaccgaagcagtggttaagccggagggctcggaagaacggcaccttttctttctcgaaaaagttatatgggggctgaatgagcttctggaggcttgtttaccgttttttattgtcacacagaaaaagaaactgccttgtctcccttccgggaattctctctttaagactgtaagtcgctgcctgagtggtttcattttgttttgtttttctgcccttctctttcttcttttgccctttcttagcttgcactcccatggtgatttctgcttggtctcctgctggggttggtggtactcgttcccaccgcacagaacccggcgcctattattggccaagaaacttgagcagcctgttttgaaaagtccctcgctcagaaatgccagcttgcagatggctaatcaaagagacgtggatccaggaggctcatttgagtaccggagcctctgagagtccatggatttttattttattgggccccaacaaagtgccaggcatagcgagagaagtgtttacaaatgtgatctccgcggattctcacgagagccatgtagtggggaagataaccacatccccattttacagatggcgaaatgaggttcagagagcttcagtaattcagccaagatcacacagctagtaaatgggagatatgggattgaactcagatctaattccaaaactcaggctgttcgctgattgcatctgatatccagattacaccaagaggctgagatgtctgggtccattttatgattagaaaaaaaaatcctgttcctttcagaaatagtgtgcactatttgggattctttagaaacttccttggagaaaattaatgattcaagattgagaaaacatttatacaacctcaggccacactcttctctctcttgttattaaccattgcaccactgcaatgttggctgctttgaaaatctcatagtttttggacagcagtattagtaaagtgtaagaaaattgtcaaagcttggagctatgcttgttgaacttttgtaccaatagcacctttaccagaggctgcgtgtttaaagcccgaagaaaactttgcttttctgaaagaatacacacacacacgcatatgtaaatattcatacatttatgtatatatacatattataatacctataagttaggtataacttatatttgtatatgatatatggcctaggaaattaaggcttattaaataaaatttataaatgcagatgagtcaaatacaaagatcagacataactctatcacctaagtaatcattgtttacgttttgcagtttatcttccatttctcccctctaatatgactcttttaaatttagttacacagaaattggttttgcgctcacgacatgcctaacatcaggcctttatccttaaagaagtttcaacactcttgtgtcacctcatgtgtctacagaaaatgtttgcttttatgccttcacggttatgttttcttagtagtagcaataaataaaatagatgcaaagtgctaattacttggaatacagaagcaaattttaattctattaatgggctggacatagatgggtggggagctgttttctgcttttcccctcttcccctagtcctctgactctggtgataactatagtcaacaagtatccccatccttaaccccttttgcttctctttctctttccttctctcaaccttcctggtgttagttttctctttcctatttctgagtgtttttactccgacacttacaataaaaaagtttgaaatcccaaaaagttgacaaatttctaaaggagacagaaaaagagtcaaacttactttacaaattaatttgagaaatagctgtgggttgcattttttttttttattgtgcatgaccctgttattggccaagaaacttgagcagcctgttttggaaagtccctcgctcagaaatgccagcttgctaattgaagggatgtggatccatgaggctcatttgaattccgaagcctctaagagtccatggatttttattttattgggccccaacaggttttgtcttgcattatttaggacattggccaggcaatcactggacagccagtttgctctcctttacattttgtagaatggttatcttatggtcaaaggaaagggagagggcctttggaaaataaagctgtgtatttacaagcagtcatacagaatagttttgtggggaaaataaagaacagttatttaatttgtaatttaatcatttttcttaaaccaacccaaagagcataaaatggaatatttatttatttttgtagcttaatttgtaatttaataatttttcttaagccaacctaacgaacatattgggctgtttattttcatttttaatgggccatatttacatcacaaaggacttgagaagtcttacaaatacaataaaatgataaaatatgaattaagacattagcaaccaggatggaaaaacacaaatttgtaaatgatggccaggtttgcaagcaaaaattaaattgataaatggcagcacccccactaccagctatagttagactgatgaaaaatcctctaatgttcatgcctgaaatacagttaccacccttagcagcaaattagaggcaaagaagttctggaatttggacgttgtctctgagtaagctctttgagacactggacaagtaatttccttctgaatactcttatactgacacataggagaagtgtcagtgtttcaaaggaatctctttgaaggctactacattgctttaggggtgccagattaccagagtaaactgccagtttgagagaataactgtttcttaagagatggtctgaaacctaagtaaggaaatccccccacttgaaagcccctaggaagttaattgcttttggcaaattatcttgggccccaaaagtcccttgatgagcacatgttttgtaataggtactaaagactatttacttatgatgaaaaataaaataacacaatttaaaacttgaaattgataaaaagcaacttgaaaccaaatttaggacagtgaacccatccctttcttatttttaaataggtaaatatagaaaaaatgaccacatgatgtgaaacaaaaagcaaatatttaaaaactacatttatactttatctcaccacactaatgtaaagaattttgattttacttataaattcttagccttgtctacacatatacataattgcatgtttaatttgagctatatatagtctgttttttgttcctttgcatgttacacaaacctttttctatttctacataatttttatctcatattttttattggatacattatagagagagcatttattaaatgagctgggattactataatttttattatttattttttaatttattacttatttttataatttactattgaaaaaaagcatttcttaaatgtactgggatacatatatatatatgactttttcttgttactaaaaaattactcatagaacttacatattaaaattttaggacttgtgatgaaaaataataaaataattcaaaacgataaaaagcaacataattttatatgttgtaccttgtacttccttataaggtacaacactacaatagccttagtgtgtagttttctttttgctatgtatgtatatatatattcataaaagtggaaaaataacatttaataaatcctctttattttctggaaaatccttgctttaaaaaaagtctttagctttttatatagttctacttctaacaccaattaaatatcttcaattcagtctcaagcaggaacaaatcttcagtccacttgcctacctttactgtctttgaagtgacagtgtgagtagtaaagcccaaatcagagagaaattgacttttgtaagattggtaacttcaccttccccggaaggaaatgaggtggttcttccctttgatcacaactgctttcttgatttgaaataaactgttgtacagcccagtcaccccatctttagacatttagaaatggcaatcatagttatataggacaactagcctgagtcccactaagtagaaatgatagtgaatttagtataagattatacctttaggctgggcaggtgactcacacctgtcacccagcacttcaggaggccaaggtgggtggatcatggatcacttgagattaggagtttgagaccagcctggccaacatggcaaaaacccaactctactgaaaatacacaaattagctgggtgtggtggcgtgcctgttatctcagctactcgggaggtcgcggcaggagaatcactggaacctgggaggtggaagttgcagtgagccaagatcatgccactgcactccaggctgggcgacagagtgagactccgtctacaaagaaaacaaagaaacaaaaaatcatacctttagagcaaggccagccttgtgaattagcaaatctgactacaaattcaatcagcagatgtactcagcccagcaaagtgagcaagcttactataggttgtgttaaggcatggactgggataaactggcaaaattagagtaaatttgacaatttgacattggattcattaatgatagcctgtccttcaatgaagtcaggcagcattgtgtttctattcacctctgtgttttgtgaggctataaaaatataagatatgagaaatgtgaatactttttatttgtgagggcagatatcttgaaaaagagagcgagaggaagtcagagtatgcgggtgaagactcttgtttttttgctgaggatttagctttggtttttagccattctcacagagatcattggtcctaattaactggttgttgttgttttttttcatgtgaatgtctgtaaggtttcaggcttgattttaaaaattgggcctatactcacagaaagacaaatactgcatgatctcgtttatatgtggaatctaaaagttttgaatccataaaagtagagagtagaatagtggttaccagaggctagttgggggcaaagttgggggatttagggagatattggtcaaaggatacaaaatttcagttaggaggaataagttcaagagatctagtgtacaacttgatgtctatagctaataacaatgtattgtatacttgaaaattactaagagtagattttaagtgttctcgccacacacacacaaaagaaaatgataagtatgtgaaggaatgcccaatgtaattagctcgatttagctaattatcttccaaaatatgtacataattatcttccaaaatatgtacataattcaagacatcatgttatataccataaatatatataatttttttatttgtcgaataaaaataatacaaaaaaaccctgggcctatactgcccacaggtagtggccacaggcagagctggtcccaagcccctccctctctctgtagtcccttatcccaggttgctttattcttgtatgacccacgtggcccctctgggcttgtaagggctctggggctgtattttaggactctatagcccttacattacccatttcctctaccaaaggtcatttctcttatcttcaagcttatttttggaagggtcattatttcttcatgagaagtcaaattctgatttgcaagtgctgaacacagatgccattagtctataaaactcttatattctcacatgtaaatgtagtaacctgctaacctcagattctgcttcctctaacctagttgccctgcaactgtgatgaacctcaaaatcaccaagggtacttccccattctcagagatcctgattccttaggtctatgtaggggccttagcactttttattaaataatgcatcactacaggattctgatgtctgtcggggtcttgagaactatttttctgactctagcactaaactcaaaatgtaatttcagatggaatttcctaggctcattcattttattttatttatggcttggccagtcatggatctaatcagtaacttgcaactgttgcttcaactacctctgtcctggactttccagagattggctttaagaaagaaagaggacatccctgggataagattaccatgtgtatttttatgtacagattttgaaaaacatacaaatgaatgggatctttgcttatctcttccatgtagaaaaatttattttacttctatttcaggttttcaataatgtagtaggacaagatgacatcactatatccctgttttttacacagatatattagagatgcattatcttattggaaattttaaactattcaataataaactattagtcttttagaatttctttttcctcaagtgttgataagtaactgaaagtctacaagcagacaagcagaattagtcggtggaacttcatttcttgctccatttttgtcctcagcagtagagggtggtgagatttcaacccagcctagcaggaccaataagtctggaaagaagtagctctagttcatattggcacctgctaattctagttctgaagaccatgggaaatctcttgcctatttagctcagtgatcatccaagatgggaaattcttgggctcagctgtgagggatcccaagtttatatgtaacactaatgtctcacaaatcaagaatccaagagaatcaagagactatagttcattccttatattaaattcttaggactgccaactgggtggctacagttttgggggctagaagtctgatatcaaggtgccaactggactggttccttctgaggaccacgagggcaggatctgttcaggcttgtagatggccttcttttccatgtgtcttcacattatcttcctctgtttgtatgtgtgtccaaatttcctcttctttttttttaatttagaaatttatactgtctacaagctttatatagctttagttaaaaaaaagcaaaaaaaaaaaaaaatcaaaacaaaaacagtgaagccaagacactcttccaaagtctggacccttccggccttccaaatacaagagctctggaagttgtatataccaaatttcctcttcttaaaatgtcaccagtcacattggattaaggcttaccctattaacaccattttaacttaatcacctctgtaaagaccctatctcctgatacagtcacattcggatgctcaggaagttagggtatcagtatcactatatgaatttgggagttgggggaggacacagtttagcccatgacagtcctgaatctcctttgctagatgcatgactgtgggccagtcacttcaccagttgggatactgccatctctatcagaaaaggggtttgggctaggtcatctataaagtcctttctgctctaaattgtaaatataatgagataccaacatttagtttaattgtttaaaattttagaagctaactggatgctttgctggtgtcaacactcccctttacaaaaaaaaattcggccgggcatggtggctcacgcctgtaatcccaacactttgggaggccgaggcaggcggatcacggggtcgagagattgagaccatctggccaacatggtgaaaccccgtctctactaaaaacacaaaaattagctgggcctggtggtgcacatctgtagtcccagctactcgggaagctgaggcaggagagtcacttgaacccgggaggcggagcttgcagtgagccgagatcgcgccactgcacctccagcctggcaacagagcgagactccgtctcaaaaaaataaataaataaataaaataaaataaaataaaataaaatctcactttctgtttctactgttctctcaagtacccctctgccttctgttacttctttgctgcattctgagaggaatggggtggggagcactctttcctgtagaaccccataacccatctattttcatcaatttatttgaaattcaaagccaggggttaaaatgtccactttcaatttgagatgtctaactttcagataaatctattaacatgcatttctaagtgacttttaagctaagcattttattttattgagctcgtatggaagggtaactattatacttgtataataaagtgcactttcaaaaatactcggttcaaatgtaaaataatattcgtaatattttaagacaaacctcctagaacttatatgtgtctttcttcccagagcagtctctatgggtgcctaaacaatactgcattattagatttttttcagtttataagccacacaagaaagttaacccatgtcatctctagaggcataaaaatagacagttgttctgagaaacatatctgtttattcctagttcagtaacagcgcaatgagatcctaaattatgaggtactttttgtttatcttattgtctcattggcaaccagcaactcctgttgcatttttgacatgggaatctatcagggtgtggttaataaagtacatttccatggtaagtattctttaagaaaaaataaaccaagatttcatcagagacagaaattactgcaaactgtacttaccagtttctggtgatttgtataactgagctttcaatttcaaaaattaaaaagttctctttagtgtaacatatttttgtgtccagtaattttacttcaaaaataaaagagaattttctttaccatcaaacaacgttgctgtaatgagtggtgatatatttagtcctagaggaccaaagagaaataaaatggctttgattatgctggtttaagaaaatatatccgatgaaacagaggttaatatttgtgatgtttgatttagtaaaattataagactaccaaagaaaactagaagagcaaaataaccaaaatttatataaagaattcaatgttgataattcaaaggtttagaattttttccacctttcaaatactaagttgctgttatagatttactaccattattacccagattttctattttactaaaaaataataacacagttcgttatgataaatcagtatagatctttaggctcaacacaaccctgctaccttttaaacttttttattatacaacaaacacatatgcattatcaagaaaagatgagtttagcaaaatgaagaaaaatagtcttatcaactagaaataagcaccattaaaatttctgtgtgttaaaagataatatgctgctgaaactttttttttcttgaaatgtccctcttttctttcacttttcattttcttaatccctaaccttctactgttgaattttctggaccaccgttataccccctcatcaatagggcatttttttctgcagtagctgagcacaatggtcatattgaagaaattcccttgtgggggctttgcctagaggtaaaatcctcttggccgggtgcgttggctcatgcctgtaatcccagcactttgggaagctgaggcgggagcatcttctgaggtcaggagtttgagattagcctggccaacgtggtgaaatgccgtctctactaaaaatacaaaaaattagccgtcatagtgtgcattcctgtaatcccacctactcgggaggctgaggcaggagaattgcttgaatgccggaggcagaggttgtagtgagccaagatctagattgtgccactgtgctccagcctgggtgacagagcaagactccgtctaaaacaaaacaaaacaaaacaacaacaacaaaaacaaagtaaaaccctcttcactgtgatgactctaaagcttgcttttgtctctctaaatcacaacaaaccgtttgcagtgtgatgctgtgctcaaaattgagaattctcactgactgttccaggcatctgtctactcattcttgccatgctgagggctgtcatttctcaccactgaatgcctttttttttttttttgtgcttctttttaaaagtcttttcctgtttgctttgaacttcaataaaaacagggggtaagaaggtaatatgcttttattttgtctgtggggtttttttcttccttcttttggttccttttcagatcctagctcatcaaggccatttgccacactgacactggaataagcctgaatgggggtctatatttcttccagtagttagtttaattcggtcattgagctgttgctctatttgtaaccgtcatgctagatccgagggctgactattcctgagaaagtgtggagatcctttcagactgccatggtgctagggcagagtttcagtcattatcttctgtgtctgctaattcttgcatcatttcttctactctgtaacctggttggctatgaaaagtgattcttcctcactgatgctgcaggcaatgggtgaatatccaattgttctctcaggttcttgtcatcggatacaaagaacaaatttcatgtgtccctgtttctgagaggttcaaagggagcaaatgcacagattctagcttaggatgacttccagcttctaactatttgtctgtgcattccatttcctcacccctgcgagaggttgcagtcatggtaacggctgttctgagaaattcagtgcccaaactggagggaggggaacacaaactcagcttcttcccaagcttcagggcagggctggcaagttttgccttccccgccagcaagagccctggctttaggggtcttgctacagttttcccagactggaggtcttggggctggattctggttcttagcgaattcttgccaaacacctcccctttccccagtatccatttgttttgaaggttctttgcaggcttcttgccaataattgtcttaaaacaaatggagttgtttgcatacctagatggagtaaaatatttaggaaactattctacaaatatgaaacagcccagatttgggccagaagtcctggtcctgagtctcttgttagccactcttaactgtgaattattggacaaatcatttctcgcactctgtgtaatctgtcacctgggtacttgatgatttaaaccaggaattgtggcccttctcataatactgtctcctaaatattgggtgaatttagtatatcaaggagggcagggcactatacccgtaggggactctctctctccctctgtctctttgcctttctcagatatgcatctttggtggcagacctggggttcaaaccacagttgttttgaaatttctctttccttctccactatgctccccctcactgaaactttaataataaaagtagcacacttattgggcatgtatttgttatgtgccaggttctgtgctaacagctttaagtgcattattccacgtattttcacaacaagcctgtgaggaagaggttatttccacttatagataaagaaactgagacttggagaatgtaagtgattttacccacaaacacagggcagtaagtgggaggccctgttcaccttagaacacctggcttctatcattcatggtgctgtttctagtgtggtttgaagaacctgagatccaaactgctatacaagtgacctgctgctttcttggagagagaaatctgaaagacagtggagccctcacattgtctttgcctgtgcacagcagatactgccaattttgggtgggttacacttgtttaccacgttgcttacttcagaaatcaataaaattctcttcatgcttttattttacaggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtatgttacacaaaacatccagagattacagtgaaagtcacagttaggagtagcacatagtaatcatgactataataattttacagtttttggttcccctatattatataacataactgagagaaaaacaactatgaaattattttccaaagatgagttttatttatatttatcacgcttatttgatgtggttatggataaatttaatttacaagtgacatgcacctctgaaatgagaagactggtctatttggctccatttttttctaagcaaaaatgactcatttgtgaatatgaaagcttatcatgtcaaaactgattaggatctaccataacaatagtggaactctgaaagcaagagacttctcctcttaagggatatgtctcagcacttcccttcaatcagaaggtctctaagaaagctgtcctggcactgaagcctgcaaaatatatttgcattttgcagctgaataactagacgaataagaaaagcaacaggcagaaaccaacagcttttttttttaaatcaatactttgaagggcaaactaatttcagatcacaaatccttcaaacactggtaaattagaagctaagcaaaatgcaattgtgcaaacatttcttcaactttgagtagagttgaaaaaaggaaaatagcacatgggttcatgtgatagattaatagaacaaaaaaggactgtagtatgtttcatggttgtcctcccaggcttccagcactcaagctgtaaaacaggatgggtcctgtttgtgaaaagattctaccagtctccctgtgcttcatgtccagattttaataaccgttatggaaaaatctcccctataacccaacccatttatacttaaacccatttacacttaaatcttctccaacacgaataaagaatggcgtcctggctttattataacctgctaaacacaatcaatttgctctatgaattgaaatctttgttcataattttaaaacctaaaatgtaaattaaaatgaacaaaatatgctttgcacatcaaactgtcaaggatttttgtctattgctttttcggttttgctttgttttattttaatttaagaagtgaatgtaaattgttacaaacttttaagaaatacatttggatatatgagtcaaaaatttgaaaaaaaattttttttaacttttttttcttattattatactttaagttttagggtacacatgcacaatctgcaggttagttacatatgtatacatgtgccatgctggtgcactgcacccactaactcgtcatctagcattaggtatatctcccaatgctatccctcccccctccccccaacccactgtttatacttgctaagatagcaattaatttatacgaaaataccctgagaaaataatcaaaaataaaggaaaatattgtacacatgatatttcattatcaatatgatgtaggattccttaggtaaattctggaatatttaaagactgaaatagtatgtatcaaagtcatgttttctgatatatttctaatataatcctcaattatataaaccttcttaagtaatttccaactgatctggctggattaactgattctccctgatccgtctataaaatatcctggatggcatccaaccatgttaaatactcttcccacttcaattccctcttgttgaattttaggcttatcaatatctaagttgcttccaaatctgtttatgctcattagtgttgtgatattcatatgtaatttgattgaatattgcataaattaatgaaatacaaatacagagaaaagacagattttatttctaagaaaactaggttgagtgctttggagaattcaataaagatgagctgctaaaatgttgttgatgaattttgtgagggaaaaataaaagttaagtagtaggaaaaatcacaaaaatctctaaagccattcatagagattattggtatgtttatgttttcctttcatcaaaaacgaagggagggagggagggagggagggaggagaaaaaaagaaagaaatgaaaaaggaagagaaaaatagacaaacaggaaattataggtgatgcattatggatgtgacttaagaaagaacctgggattataataacttctatatcacaaaattgtcaaatgattatatagttatattttataaattaaaacaaaatatttacagtatttttaagtgatttcctatgtaaattgacatttttgattaaatgatcaaagaagtcctccccacatcatcatattcaataagaaggattctatggtatactgcacacatatttcatatatacacattgtacatatatatatatacacacctgttatatatatgctcatgcatatacatgtatgtatgtattatatgcataggaatgcaacaaaataacggcaatatctccagattgttgatatatagttttttgtttgtttgtttgtctgtttttttgagatggagtctcactctgtagtccaggctgaagtgcagtggcatgatctcagctcactgcaacctctgccttctgggttcaagcaattctgcagcctcagcctcccaagtagctgggactacaggagcacaccacaacaccgggataattttttgtgtttttaatagaggcggggtttcaccatgttggtcagtctggtctcaaactcctgacctcaggtgatctgcctgccttggcctcccaatctgctgggattacagacatgacccactgtgcccggcctgatacataagttcttattatactttcctgccttttccaacatttctataataagcatatgtaccattatactaaaaaaagtattttaaaatctgattttcaataaaacaactacttattttcttattttaataaattttgttttcatactttggaggaacgaaagaacctgtggtttactgtatctccattataccaaggaagttgtgctaggcacattctgtaagataattcattaaagagaccaattcattcagagaccagtataataccactagcaaaaaataaaattatacttggacttgggttctgttttctttaaatattgctacaatgttatatagccctatgcactatttttgaattttgttctgggaatctccagtttgttttttactattgacagactattatctttacttactaaaatagcatatatatttacatgtcataatttgctcatgtagtaaatattttgttttatcaaaaagaatatatgaagttattaaggaattcatgttcctaggaaacacagcagaaattaattttgctgtataggtgagcagagcagattaaactgcttatatttttaaaactagttataccattaacaattagagtcactagtaatgtttttctcacacttcccttgatattttctgtcactttcttcttaattctctattcccttaccttcatctagctttccccatagcatttttttagcacttgatatcaatatatttatttgtgtattgtctgcctgcatcccaagaaggtgaagtctttggtgatgaagggtttatcttatttcctgcataatatcaaacttctaaaaagttcctaggactcaataggcatttaataaacacctatcagataaatgaattttttaagagaacacattacataatgctttctaatagcaatacagtgacaaactttatttttaagtaaattaatgcaaatatgtatattatttaaaattgtttgtttttaagataacaagcctgatagataataaattttggcataaatatgttaatgcttatcaaaactcatcagtacatatcctgtcttttgatgaaaaagtaatagaaaggggagaacaaagagaactctcatgctcactaggcaggccgagtgcagctctgacagcaatgcaagagaaatattactatctccatcatatgcaggatgaaatttaggcttggagaaaacaacacttcgttaaagattttatagctgctcaagatttgaccaaggcttttaaaacttcaaatgttcttttccttattacactgtttcttatcaggatttgtggactttggaaagtaaaatgaaaagtcagagcaatgtgagttagtttgataatctcagtggccagtaacagctatccccactctgaatatataccaaccaacctctcccctttctccaaggaagttagttcttggcaaaggcatttcatgcagtgtactttgtcattctaaccttcctttttttttgtatgctcctgttgctaatcatcctatttctatgggcttcagtcactcttttagcttttgtcttgggagactttctgtctgttgacatgacttttaaaataatcttctcccccattgtatttatatctcattagcctaccccccctccccttgtgttttagaagagttttattgtctgtcatccctctatagttcccaccctgttacctgcccgtgtcctgttcaaacacttgctccttttttccttgggcaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggtaagagtcttaaaatgcaattgaaagaggccaatcttggaatttcatgtagaaccatttataagacaatttgaaattggggcctactgtggtgctatgttgacacacaggaaagggaaggacaggtggctagggtaccgcagaaccaggtgccgagctaactactggtctagacctttatgagtaagtctaggcaattcttccagatataggagaatgagtaaatatgaaccctaggaacagggttcatcagctcaaatcaaaagctcagaaattattttttttctggccttgacttacgcttatataatggtgctcgttcatggccagaaaaattcagaagcctgcagatacaaggacacagaaatccaaaaggtaggtaggaatgctccccctttctttggggatttagtttgtcctgataattccatctggggacctgaattttcatggatatctcaatgtattctaaggacccagattgaagtataacagaagtgtttctagttttgtttgacatgaagaaacctctggacataaagcttttcccatccctattcagcctataaagcaatgtttctcatccctggcagtgcattagaattgcttagggagctactaaaaatgtttcatgtttaggtcccatctcagggcaattacatcagagtctctgagtgaggaccttggtattggcctgatggagtaagtctggggtagagcctaacattctacatttttaacaagctcccaggtgatgctcatgctgccagtccccaggtcaccctttgagtgttaaggctagagactagaggaacaggggagacgtctaagtattggtgaggtttgtcttggttgctgctaaatgattgctggccatttcaacctggggtttcctgttctgtgtttaaacactgactgtattactggtgtcatgctgtgactgttgatataagcagtggatctcaaaaatccatgcagctcctgcccaccattttcatagtctgcttataattagccgctataactaatagtttccaaactgattttctaggcttagaagtggaaataaactgcacccggacccagaataccaagtgcagatgtaaaccaaactttttttgtaactctactgtatgtgaacactgtgacccttgcaccaagtaagttttagtctttctctgattaaaacactagatataacatgagagttatcattttcctagggaagtaacactgactgagagttaagaattagggttctagtcctgctttgcctccaagcaactagatgactgtttgctcatttaaacactggtgaacttcctgtgtaggtgacactttgctggccacttaagctaaagatatgagccagacatatgcctggcatcaaggtatcagtccagaagcacagacagtcaaggaaagcaatggttattgtgtttcaacagcatgagaatagcatcagaagagacttatgttccaggtgttacaaattatacagaaaaacttttcaactctgcctgaggagatggaggtgaagacatgaatgtgtctcatgacttgaatcttgaagaaagaaaagaaagagcattaacaggtgcacatggatggaatgcacattataggcagggaaacagcagctgcaaagagacaaaggaagctcaggaaaacagaatacctggctggagtgtaggaaagactgagaagcgttcagggctgaatcatgaagggccttgtcttttaggctaaagaggttggttttgacccaaagttagagaacctgccttgtaagtcatcttgggaagcttttaggagttcatctaataggcttaagaaataagcacatgatgccttatgttttatttgctaggaaaattttatgtacagttgtcccaggctctcccctggccacaagtgtctgagagtgcccctatgatgacaggctaggtaggcattggggaagcagctgacttagtaaaataataacaataatagtaattccaaatatttcataatcttgatttaattttcttgtcatcataaccttttgaaatagataatattatttccatgttattgatgagataaaaggctcatggcagtgtgacttcccaagctcacctagtcaggtaatgggcagaggctgtgaactcagacaacctgattgtgaatgtttgtctgtctgaaggaaatcacacatgaacctcttgagtctcctgatcaccaccggttgctaaaagtggcagcctctaagggccagctgagtaccctccctgagctacatcatgggcgtggctatcacctggccattttcttggtctataggaattttttgaaattacttttgacagtttattttaagagctagtttaagctataggatttacgtgttcagtttattactaggtttaagtttatttttgtatccacttcatctctcttgtgtgtcactattttcctatcttcctttaactcttgaaatcttaagacagagtcattccttatgatatttttcatccagccatccaaattatattaacttgtgccagctttagatactaatttagaaatatttgaaggaatacgtttgccagagatgcaaagatgaataaaatggcccctaatttacaaagtgccattgaaaattataaaggaattattctgccaggcttttgaatttctcctgtatttttttttctagatgtgaacatggaatcatcaaggaatgcacactcaccagcaacaccaagtgcaaagaggaaggtaattatttttttacggttatattctcctttcccccaaccccatggaaagatgtgaagaaaaaccaatcactcttgattactagaaagtcctttatttaatcttaaagattgcttattttcatataaaatgtccaatgttccaacctacaggatccagatctaacttggggtggctttgtcttcttcttttgccaattccactaattgtttggggtaagttcttgctttgttcaaactgcagattgaaataacttgggaagtagttcacaaagatttgcctcattcttacctataaaaagctaccactttggtagatttatgtattgttaatttcttgcccctgaatgcagccttgagagctgactgataagaacaaatgaaattattcctcagctagtttctgagcaacagttttggggcattgagtggtattctcatccttcctatgaacaggtgttctctgcagcagcagaattggccaaaaatcagaagcaattcttcactattcattgagatctccctatgcaaaaagagaacacaagaagcaaaggcattcccaggaaacacattgcagggaacactttaaaaacttgtacttcactgcctcctcttcctcggcctaattgcttgtttttaattatttctccttcttaacttaaaatactatggggacacatgttatacaaaggtgacttagtagagtcagtagaaaagccaaaattagatattatcataattagtctagaaaaatccctttaagtcattcatcaactacagggtcacaccaactttcagtaacttagaagtattcaattttcccttctcagaacaattatctgtttcttcagttcagttgaagaagaaagtttgccttgcctttagcggttgtttagctgaaaatacatttgggatatttaagcactgtaattgtgctcagagacatacagattcttctatctcacattgactttaatgcatacacctattgagtatgtatgcttgagttatttgtgtgtgtatttcatttctgggcatccatagcaagttgatgttgacttgcttgtcctacggcttctgcatcctgccatagtcttgccgtccacatctttgctggacagagagtggtgcttgccatatggtaagtcaaaagccatctccttgctaggccagcctgtggtaattagatgactaattaagatatgtcctttcactagaacacttgacttagtagtacgaaagttccaaaatcagcggtctcctgcgatgtttggccacttttaagtttcactgaatttctcctttttccttcttatatttctcttagtgtgaaagtatgttctcacatgcattctacaaggctgagacctgagttgataaaatttctttgttctttcagtgaagagaaaggaagtacagaaaacatgcagaaagcacagaaaggaaaaccaaggttctcatgaatctccaactttaaatcctgtaggtattgaaataggtatcagctttccttgaaaagaaaaatagagaaattagtgatttggctttttgttacttccttttacttttttgtttcttgtttgtttcattttgtttgagatggagtcttgctccatagcccaggctggagtgcaggggtgcaatcatggctcactgcagcctcaaagtcctgggctcaagtgatcctcctgcctcaactgtgaccctgggactacaggcatgcaccaccatgccaggttaattttttatttttattttttatagagacagggttcactttgttgcccaggctggtctcaaacttttgacctcaagcaatccttctgcctcagcctcccaaagttctgggattataggtgtgagccatcacatccggcctgttactttctttatttaaacattgcttgtgtttgtgggtgcataggttaaaggggcctcacagatgaattatgatatggtacccaaattaaaagtaaacttgtacacaaaaaccgtaagaatcaacaaaggagggaattgagagtgtgctgaaaatgagctgaaaaaagaacttggccaacctccatgttccgtgttgttgttatccctgcagagaaacagcctttaccaggcatgggttcagtatccaagcctgagcaaagagattgctcttaaagtttgtcataagggcacagcttaactttctctggctgccggttgaccttatgtctcaccatatcttttcccaacatggtatcaatatatttcagatagcaagaaatacaccgagaaaagaaaccagttgcacttaatggaattattttcttatttggggccagggctcagtgggagttaggattctttaaattaatatggtaaaaaaaatttcaatttgagccattttctggtattaagaataagaatagttacagggaagaaaattttagagtagttatatttattatttattttaataagtctatatgaaaaataacctcaacctattttatgaattctttaggtttcttgcctttaaaaactaagacaatattgcttagtttctggcaaggccggaacctttcagaataaaaattgaatggtaaaagtaaccttcttaatcacttaatctagcttcctaattttatacatcaagcaactgattgtacttctttctgaattaaggaaaaattagaagttcacatttagaatattctaaagatatatttttatttgtctttctctgcttccattttttgctttctaggaaacagtggcaataaatttatctggtaaggcttttatcattttatttcatagagatggcatcctttagagtaataggccaatttcagagtaaaataatgttactaatttcagtgacatattatgggatcttgttatttctcatacaattctacctgctcagcataaagcatttatcaggcagtttgtttaaatttataatgaatactcatagttaaaaataatcaagtaacaataagacacaatagtctgaggcttaagaaacttttccttcataatcagctagatgtattacagaactcctgcctaaaaagatctagaggttaaagtgtactgtagactcaggtattatcagtgtacccaactctataacaacatacatgattccattcagttgttctttgatctgtgatttagagataagatgatcataaactctttgcttatacttttagatttgtgggtcattgatcattggatccataagagatgttataggtgatcttttccaatcctcttaatttataaatgagaaaagaagatacagaaatgttatgtattgtggcccaggttatttaacaaattactgggtatatggcaggatttggagttagaactcaaggttgtcttgtttctgtattcccctagtcagctcttcatagacctttaggacttagctatattctgaagtactataaaaagagaaataaacatggttttcactaatgggaatttcatttagaaaaacaaattttcagactattttctatttttcagatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttaatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcactcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacattcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaa

SEQ ID NO: 32 - DNA, 3696 bp, Homo sapiens
  source 1..3696: mol_type = genomic DNA; note = mFAS mRNA NCBI Reference Sequence: NM_000043.6
ctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttaatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcactcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacattcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaa

SEQ ID NO: 33 - DNA, 3633 bp, Homo sapiens
  source 1..3633: mol_type = genomic DNA; note = sFAS mRNA NCBI Reference Sequence: NM_152871.4
ctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaagtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttaatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcactcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacattcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaa

SEQ ID NO: 34 - protein, 335 aa, Homo sapiens
  source 1..335: note = mFAS Protein NCBI Reference Sequence: NP_000034.1
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 35 - protein, 314 aa, Homo sapiens
  source 1..314: note = sFAS Protein NCBI Reference Sequence: NP_690610.1
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 36 - DNA, 43 bp, synthetic construct
  source 1..43: mol_type = other DNA; note = Template strand of Figure 3A
caaagccaccccaagttagatctggatcctgtaggttggaaca

SEQ ID NO: 37 - DNA, 12 bp, synthetic construct
  source 1..12: mol_type = other DNA; note = SCR sequence of Figure 4B
ttccaacctaca

SEQ ID NO: 38 - DNA, 24 bp, synthetic construct
  source 1..24: mol_type = other DNA; note = FAS DELTAEX6 sequence of Figure 4B; note = Wherein N is any nucleic acid base (or, wherein N is A, C, G or T)
ttccaacctacannggatccagat

SEQ ID NO: 39 - RNA, 11 nt, synthetic construct
  source 1..11: mol_type = other RNA; note = mRNA SCR sequence of Figure 4D
aggaggatcca

SEQ ID NO: 40 - RNA, 13 nt, synthetic construct
  source 1..13: mol_type = other RNA; note = mRNA SCR sequence of Figure 4D
ttgggtgaagaga

SEQ ID NO: 41 - RNA, 14 nt, synthetic construct
  source 1..14: mol_type = other RNA; note = FAS DELTAEX6 sequence of Figure 4D
aggaagtgaagaga

SEQ ID NO: 42 - DNA, 20 bp, synthetic construct
  source 1..20: mol_type = other DNA; note = mCRISPR_AS guide sequence
acaaaatatccaatattcgc

SEQ ID NO: 43 - DNA, 96 bp, synthetic construct
  source 1..96: mol_type = other DNA; note = mCRISPR_AS sgRNA full sequence
acaaaatatccaatattcgcgttttagagctagaaatagcaagttaaaataaggctagtccgttatcaacttgaaaaagtggcaccgagtcggtgc

SEQ ID NO: 44 - DNA, 44 bp, synthetic construct
  source 1..44: mol_type = other DNA; note = FAS DNA long target sequence: (target site) EXON6intron5 antisense
ccataggcgatttctgggacctgcgaatattggatattttgtat

SEQ ID NO: 45 - DNA, 20 bp, synthetic construct
  source 1..20: mol_type = other DNA; note = FAS DNA short target sequence:
gcgaatattggatattttgt

SEQ ID NO: 46 - DNA, 20 bp, synthetic construct
  source 1..20: mol_type = other DNA; note = mCRISPR_KO guide sequence
cagttaagagttcatactca

SEQ ID NO: 47 - DNA, 96 bp, synthetic construct
  source 1..96: mol_type = other DNA; note = mCRISPR_KO - sgRNA full sequence
cagttaagagttcatactcagttttagagctagaaatagcaagttaaaataaggctagtccgttatcaacttgaaaaagtggcaccgagtcggtgc

SEQ ID NO: 48 - DNA, 48 bp, synthetic construct
  source 1..48: mol_type = other DNA; note = mCRISPR_KO FAS DNA long target sequence: (target site) - EXON2 antisense
gctattagtaccttgagtatgaactcttaactgtgagccagcaagcac

SEQ ID NO: 49 - DNA, 20 bp, synthetic construct
  source 1..20: mol_type = other DNA; note = mCRISPR_KO FAS DNA short target sequence
tgagtatgaactcttaactg

SEQ ID NO: 50 - DNA, 20 bp, synthetic construct
  source 1..20: mol_type = other DNA; note = mCRISPR_SCR Guide sequence
atatccacctaatcgaataa

SEQ ID NO: 51 - DNA, 72 bp, synthetic construct
  source 1..72: mol_type = other DNA; note = mCRISPR_sgRNA full sequence
atatccacctaatcgaataagttttagagctagaaatagcaagttaaaataaggctagtccgttatcaactt

SEQ ID NO: 52 - DNA, 20 bp, synthetic construct
  source 1..20: mol_type = other DNA; note = FAS5to7 F:
atgcacactcaccagcaaca

SEQ ID NO: 53 - DNA, 19 bp, synthetic construct
  source 1..19: mol_type = other DNA; note = Total_FAS F:
tgcagaaagcacagaaagg

SEQ ID NO: 54 - DNA, 24 bp, synthetic construct
  source 1..24: mol_type = other DNA; note = Total_FAS R:
tttattgccactgtttcaggattt

SEQ ID NO: 55 - protein, 1368 aa, Streptococcus pyogenes [sequence omitted]